(19) United States
(12) Patent Application Publication
(10) Pub. No.: US 2003/0082511 A1
(43) Pub. Date: May 1, 2003

(54) Identification of modulatory molecules using inducible promoters

(76) Inventors: Steven J. Brown, San Diego, CA (US); Damien J. Dunnington, San Diego, CA (US); Imran Clark, San Diego, CA (US)

Correspondence Address: David B. Waller & Associates, 5677 Oberlin Drive, Suit 214, San Diego, CA, 92121, US

(21) Appl. No.: 09/965,201

(22) Filed: Sep. 25, 2001

(51) Int. Cl. C12Q 1/00; C12Q 1/68

(52) U.S. Cl. 435/4; 435/6

(57) ABSTRACT

Methods for identifying an ion channel modulator, a target membrane receptor modulator molecule, and other modulatory molecules are disclosed, as well as cells and vectors for use in those methods. A polynucleotide encoding target is provided in a cell under control of an inducible promoter, and candidate modulatory molecules are contacted with the cell after induction of the promoter to ascertain whether a change in a measurable physiological parameter occurs as a result of the candidate modulatory molecule.

### TECHNICAL FIELD

The present invention relates generally to the technical fields of molecular biology and drug discovery. More specifically, the invention relates to the method of identifying a drug target modulator using an inducible vector.

### BACKGROUND OF THE INVENTION

Advances in molecular biology have increased the efficiency of gene isolation and sequencing. Additionally, the availability of known sequences and sequence alignment programs allow comparisons to be made leading to the identification of motifs that are conserved between members of the same family or similar classes. This allows genes to be assigned to particular target families, such as G-protein coupled receptors or ion channels. However, in the case of receptors, sequence information of the target does not provide the identity of the receptor's native ligand or that ligand's biological function. For example, single transmembrane membrane receptors contain a cysteine rich domain, followed by an alpha helix motif, followed by a tyrosine kinase domain. This may suggest that the sequence is a receptor, whereby the cysteine rich domain is involved in ligand binding, the alpha helix traverses the membrane, and the tyrosine kinase domain is involved in cellular signaling. Unfortunately, sequencing an unknown receptor's ligand binding domain does not provide sufficient information that would easily lead to the identity of the ligand. Similar problems occur when searching for the function of ion channels, enzymes, transporters, transcription factors, nuclear receptors, chaperone proteins and other regulatory molecules within the cell. Consequently, experiments must be designed and performed to identify the sequence's function and modulatory compounds.

Controlled expression of the target sequence is necessary to identify modulatory compounds because constitutive expression often leads to over expression of the protein. This is frequently toxic to the cell or can cause down-regulation of the target by stimulation of internalization and degradation processes. However gene expression is difficult to control in terms of both the level and time course of target expression. Current expression vectors are usually designed to maximize expression levels, and therefore yield cells that continuously express the target. Alternatively, techniques such as transient transfection reduce the target's duration of expression, but these techniques often lead to heterogeneous expression among replicate samples, are labor-intensive, and may damage the cells or alter their function due to the need to penetrate the membrane to deliver exogenous genes, making data difficult to collect and analyze.

The activity of a compound against a target of interest is determined by a variety of techniques. Some examples include randomly screening the compound against cells transfected with the target, testing compounds in cells where the target has been mutated to express the protein in its active state, and binding studies between a compound and an isolated form of the target. However each has problems associated with the technique.

Random screening of transfected cells requires a number of assumptions that often may not be tested. It requires the target protein be properly expressed, correctly localized within the cell, functionally coupled to a signaling mechanism, and expressed stably throughout the duration of the testing process. However, when the function of the target is unknown, these requirements can not be tested.

When the target is a membrane protein such as a G-protein coupled receptor (“GPCR”), it may be mutated such that the protein is expressed in its activated form. Since ligand binding of the mutated protein frequently causes a drop in activity, an assay that detects a drop in activation suggests the compound binds the target. However, since this technique identifies compounds which bind to a mutated protein, the compounds may not possess the same affinity or avidity for a native protein. In addition, this technique is not available when information regarding the design of an activated receptor is unavailable, such as the active form of ion channels.

Another frequently used technique to identify modulators is to perform competitive binding assays. However, competitive binding assays require a native ligand to assay the compound, and as previously discussed they are frequently unknown.

Lastly, assays that directly measure binding interactions using purified proteins allow the measurement of interactions between compounds and targets. Examples of direct binding assays are surface plasmon resonance spectroscopy, thermal denaturation profiling, and multipole coupling spectroscopy. However, these techniques only detect binding and are not functional assays. They do not distinguish between agonists, antagonists, or non-functional interactions. Moreover, when the targets are membrane proteins in their native form, purification is not always possible. When a purified form is unavailable, interaction among other molecules in the preparation may lead to false positives or false negatives in the assay.

Therefore there is a need for methods to assay the effects of compounds on the function of biological targets. Specifically, there is a need for an assay that allows control of the expression of the target sequence, identifies target expressing cells, expresses the target in its native form, can distinguish between agonists, antagonists, and nonfunctional interactions and may be performed within the cellular environment.

### BRIEF DESCRIPTION OF THE FIGURES

FIG. 1 is an illustration of the inducible expression vector comprising a tetracycline inducible promoter, a pcDNA4/TO vector construct and a murine KCNC1 potassium ion channel gene.

FIG. 2 is a photograph of a 1.5% agarose gel demonstrating KCNC1 mRNA production of clones 7, 13 and 22 under non-induced (“(−)Tet”) and induced (“(+)Tet”) conditions.

FIG. 3 is a photograph of immuno-staining of KCNC1 produced by clone 22 under non-induced (“(−)Tet”) and induced (“(+)Tet”) conditions.

FIG. 4 is a graph demonstrating hyperpolarization of an induced population of cells compared to a non-induced population of cells and their responses to 50 mM, 100 mM and 150 mM KCl.

FIG. 5 is a graph demonstrating that cells induced to overexpress KCNC1 when pre-incubated with 4-aminopyridine, show characteristics more similar to uninduced cells.

FIG. 6 is a graph demonstrating that cells induced to overexpress KCNC1 when pre-incubated with BaCl₂, show characteristics more similar to uninduced cells.

FIG. 7 is an illustration of an inducible expression vector comprising a tetracycline inducible promoter, a pcDNA4/TO vector construct and a HERG potassium ion channel gene.

FIG. 8 is a graph demonstrating that induced HERG expressing cells are hyperpolarized as compared with the uninduced cell population. The addition of 100 mM potassium chloride depolarizes the HERG expressing cells to a greater extent than the uninduced cells. Induced cells are also more sensitive to 25 nM pimozide than are uninduced cells.

### SUMMARY OF THE INVENTION

One aspect of the present invention includes a method for identifying molecules that modulate a target protein, comprising providing mammalian cells transfected in such a way as to provide a nucleotide sequence encoding the target under control of a heterologous inducible promoter; inducing the promoter under conditions that provide a detectable change in a measurable parameter associated with the cells; contacting at least a portion of the cells with a test compound to ascertain whether the test compound affects a change in the measurable parameter; and repeating the contacting step with at least one other test compound. Preferably, the measurable parameter is a parameter other than growth or survival. In one embodiment, the contacting step comprises contacting cells with the test compound while the promoter is induced. The method may advantageously include comprising comparing the value of the measurable parameter in uninduced cells with the value of the parameter in induced cells.

In one embodiment, the method includes testing various candidate parameters to ascertain which one is most directly or most advantageously associated with induction of the target sequence. Thus, the measurable parameter can be selected from among a plurality of candidate parameters based on the comparison.

The promoter can typically be induced to different degrees. In some cases, induction of the promoter can have a deleterious effect on cell growth or survival. Thus, the cells can be cultured and expanded without induction of the promoter, and then the promoter can be induced as part of the assay. In one embodiment, the promoter is induced to a degree that provides a detectable change in the parameter but not to a degree that kills the cell. The invention also includes empirical testing of various levels of induction to select that level that optimally provides a cell that is responsive to stimulus or provides an optimal level of signal, while maintaining that amount of viability or cell function necessary for successful performance of the assay.

Induction can occur in various ways. Thus, the methods of the invention include including the promoter by contacting the cell with an inducer molecule. They also include induction of the promoter by removal or inhibition of a repressor.

In some embodiments of the invention, the target protein affects ion channel activity of the cell. In one particular embodiment, the target protein is an ion channel protein.

In other embodiments of the invention, the target protein is a cell surface receptor, such as a G-protein coupled receptor. In still other embodiments, the target protein is another type of signaling molecule or transport molecule.

One aspect of the present invention includes identification of the type of signal being produced by a candidate molecule, or more particularly, the method by which the signal is being produced or by which the modulation occurs. Thus, the method may include identifying at least one test compound that modulates the measurable parameter in the cell; providing a second cell line that differs from the first cell line in that the inducible promoter controls expression of a reporter instead of polynucleotide encoding target; contacting the second cell line with the identified test compound; and ascertaining whether the identified test compound affects the expression of the reporter. In this manner, one can differentiate between compounds having a genuine effect on the target, and compounds that simply modulate the activity of the inducible promoter.

The polynucleotide encoding the target can be transfected into the cell, or can be endogenous polynucleotide that is simply placed under the control of an inducible heterologous promoter that functionally replaces the endogenous promoter (if any).

The invention also includes a method for identifying an ion channel modulator molecule comprising obtaining a cell that conditionally expresses an ion channel target; incubating a potential ion channel modulator molecule with the cell; and determining whether ion flow through the ion channel targets has modulated, thereby identifying molecules that modulate the ion channel target. In one embodiment, the cell that conditionally expresses the ion channel target has been induced to express the ion channel target. Some preferred cells include CHO, CHO-K1, HEK293, COS, Vero, SH-SY5Y, and U20S cells. The cells are advantageously mammalian cells, although other cell systems may also be used. In a particular embodiment, the step of obtaining a cell that conditionally expresses an ion channel target comprises genetically adapting the cell to produce an ion channel target. The cell can be genetically adapted, for example, by transducing or transfecting the cell with an inducible vector comprising an ion channel target. The inducible vector may comprise an inducible cassette wherein the inducible cassette comprises an inducible promoter, an ion channel gene, and a gene conferring resistance to a selection agent for selecting transfected cells wherein the inducible promoter is operably linked to the ion channel gene. Suitable inducible promoters include the heat shock inducible promoter, metallothionin promoter, ecdysone-inducible promoter, FKBP dimerization inducible promoter, Gal4-estrogen receptor fusion protein regulated promoter, lac repressor, steroid inducible promoter, streptogramin responsive promoters and tetracycline regulated promoters, as well as any other compatible promoter.

One embodiment of the invention includes a method wherein the inducible vector may be activated to express the ion channel target and inactivated to prevent expression of the ion channel target. As one example, the ion channel target is an ion channel selected from the group consisting of a sodium ion channel, an epithelial sodium channel, a chloride ion channel, a voltage-gated chloride ion channel, a potassium ion channel, a voltage-gated potassium ion channel, a calcium-activated potassium channel, an inwardly rectifying potassium channel, a calcium ion channel, a voltage-gated calcium ion channel, a ligand-gated calcium ion channel, a cyclic-nucleotide gated ion channel, a hyperpolarization-activated cyclic-nucleotide gated channel, a water channel, a gap junction channel, a viral ion channel, an ATP-gated ion channel and a calcium permeable beta-amyloid peptide channel.

Yet another method of the present invention is a method for identifying an ion channel modulator molecule, comprising the steps of obtaining a cell that conditionally expresses an ion channel target; adding an inducer molecule that induces expression of the ion channel target in the cell; measuring membrane potential of the cell; incubating a potential ion channel modulator molecule with the cell; measuring changes in membrane potential; and determining whether ion flow through the ion channel targets has been modulated, thereby identifying a molecule that modulates the ion channel.

The invention also includes a method for screening chemical compounds to identify an ion channel modulator compound comprising the steps of obtaining a cell that conditionally expresses an ion channel target; adding an inducer molecule that induces expression of the ion channel target in the cell; measuring membrane potential of the cell; incubating the chemical compounds with the cell; measuring changes in membrane potential; and determining whether ion flow through the ion channel targets has been modulated, thereby identifying compounds that modulate the ion channel target.

Still another aspect of the present invention includes a method for identifying a membrane receptor modulator molecule comprising obtaining a cell that conditionally expresses a target membrane receptor; inducing expression of the target membrane receptor; measuring a physiological condition of the cell to obtain a first set of data; incubating a potential membrane receptor modulator molecule with the cell; measuring the physiological condition of the cell to obtain a second set of data; and comparing the first set of data with the second set of data to determine whether the physiological condition of the cell has been modulated, thereby identifying a molecule that modulates the target membrane receptor. The cell used in the method can be provided as a cell that contains an endogenous target membrane receptor sequence and an endogenous noncoding sequence (such as a promoter); wherein the method includes inserting an inducible cassette comprising a 5′ insertion adapter, a regulatory sequence and a 3′ insertion adapter within the endogenous noncoding sequence such that the regulatory sequence is operably linked such that it is able to modulate transcription of the target membrane receptor by the presence or absence of a regulator. In one embodiment, the regulatory sequence is a non-mammalian enhancer sequence or a repressor sequence. This non-mammalian enhancer sequence can, for example, be a herpes virus enhancer or an artificial enhancer. Alternatively, the non-mammalian enhancer sequence can be an inducible promoter, e.g., a herpes virus promoter or other suitable inducible promoter. In another embodiment, the regulator is VP16 or a functional domain of VP16. One method of the present invention includes transfecting the cell with a regulatory expression vector construct comprising a second inducible promoter and a regulator gene encoding the regulator operably linked such that induction of the second inducible promoter by an exogenous stimulus initiates transcription of the regulator gene. The second inducible promoter can, for example, be a tetracycline inducible promoter or an ecdysone-inducible promoter. The external stimulus for inducing the target can be any suitable stimulus, such as, for example, tetracycline, ponasterone, dexamethasone, a heavy metal ion or heat. The step of inducing expression of the target membrane receptor can also be initiated by the presence or absence of a regulator or by the presence or absence of an inducer.

In one embodiment that uses an inducible cassette as a transfection vector, the inducible cassette further comprises a target sequence such that the target sequence is transcribed upon induction of the inducible cassette. Particularly preferred target sequences may be selected from the group consisting of a G-protein coupled receptor target sequence, a nuclear hormone receptor target sequence, a cytokine receptor target sequence, a protein kinase-coupled receptor target sequence a nicotinic acetylcholine receptor target sequence, a ionotropic glutamate receptor target sequence, a glycine receptor target sequence, a gamma-aminobutyric acid receptor target sequence, and a vanilloid receptor target sequence. One useful target sequence is 5HT4.

When repressor sequences are used, one particularly useful repressor sequence is able to bind a zinc finger protein. Advantageously, the zinc finger protein comprises a KRAB domain.

Still another method of the present invention is a method for screening a chemical compound library to identify a G-protein coupled receptor modulator molecule, comprising obtaining a cell that conditionally expresses a G-protein coupled receptor; inducing expression of the G-protein coupled receptor; measuring a physiological parameter associated with the G-protein coupled receptor to obtain a first set of data; incubating a potential modulator of the G-protein coupled receptor with the cell; measuring the physiological parameter to obtain a second set of data; and comparing the first set of data with the second set of data to determine whether the physiological parameter has been modulated, thereby identifying a chemical compound that modulates a G-protein coupled receptor. Suitable physiological parameters can include, for example, a cAMP level, a calcium level, and a membrane potential of the cell.

One particular embodiment of the invention comprises an inducible vector containing an ion channel target having a nucleotide sequence shown in SEQ. ID NO. 1, or a cell containing SEQ ID NO:1 under control of an inducible promoter. The invention may also include an inducible expression vector comprising a tetracycline inducible promoter, a pcDNA4/TO vector construct and a human HERG potassium channel gene. Still another invention is an inducible regulatory expression vector construct comprising a subcloning vector, a second inducible promoter and a regulator gene. The present invention also includes cells transduced or transfected with any of the inducible vectors described or contemplated herein. In one embodiment, the cell is a CHO cell and the transduced or transfected cell expresses tet repressor and HERG potassium ion channel gene.

The present invention also includes ion channel modulators, membrane receptor modulators, G-protein coupled receptor modulators, and other modulators identified using the methods of the present invention.

The present invention also includes a kit comprising cells that conditionally express an ion channel target, a compound that induces expression of the ion channel target, and an indicator compound or system for indicating ion channel activity of the cells. It further includes a kit comprising cells that conditionally express an ion channel target and a fluorescent dye.

Definitions

Prior to setting forth the invention, it may be helpful to first set forth the definitions of certain terms that will be used hereinafter. All references, which have been cited below are hereby incorporated by reference in their entirety.

A “nucleic acid molecule” or “nucleic acid sequence” is a linear segment of single- or double-stranded DNA or RNA that can be isolated from any source. In the context of the present invention, the nucleic acid molecule is preferably a segment of DNA. An “isolated” nucleic acid molecule or an isolated enzyme is a nucleic acid molecule or enzyme that, by the hand of man, exists apart from its native environment and is therefore not a product of nature. An isolated nucleic acid molecule or enzyme may exist in a purified form or may exist in a non-native environment such as, for example, a recombinant host cell.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns. However, as context may require, the term “gene” can refer more simply to a sequence encoding a desired polypeptide or protein, particularly in the context of a “gene” under the control of an inducible promoter.

The term “construct” as used herein refers to a recombinant DNA sequence, generally a recombinant DNA molecule, that has been generated for the purpose of the expression of a specific nucleotide sequence(s), or is to be used in the construction of other recombinant nucleotide sequences. The construct may be generated for the purpose of controlling the expression of a specific nucleotide sequence(s) as, for example, in a construct containing a viral enhancer. In general, “construct” is used herein to refer to a recombinant DNA molecule comprising a subcloning vector and may further comprise an inducible cassette and/or a regulator gene.

The term “genetically adapting” as used herein refers to the process of establishing an inducible expression cloning vector construct within a cell such that the target sequence's expression may be exogenously controlled. The term “exogenously controlled” as used herein refers to an increase or decrease in expression of a target sequence by the presence or absence of an inducer molecule or inducing condition. The inducer molecule or inducing condition originates from outside of the host organism.

The term “transfection” refers to a process for introducing heterologous nucleic acid into a host cell or organism A transfected cell refers to a host cell, such as a eukaryotic cell, and more specifically, a mammalian cell, into which a heterologous nucleic acid molecule has been introduced. The nucleic acid molecule can be stably integrated into the genome of the host or the nucleic acid molecule and can also be present as an extrachromosomal molecule, such as a vector or plasmid. Such an extrachromosomal molecule can be auto-replicating.

The term “modulator molecule”, “compound that modulates”, “modulatory compound”, or “compound” as used herein refers to any compound that activates, enhances, increases, decreases, or suppresses the function of an expressed target or increases or decreases the amount of an expressed target.

The term “modulation” or “modulated” as used herein refers to any change in functional activity such as activation, enhancement, increasing, interference with or suppression or an increase or decrease in the amount of expressed target.

A “modulatory molecule” can modulate the activity of the target molecule in many ways. For example, a modulator may act on a target by affecting its conformation, folding (or other physical characteristics), binding to other moieties (such as ligands), activity (or other functional characteristics), and/or other aspects of protein structure or functions is considered to have modulated polypeptide function. Any method of modifying the target activity is suitable for the present invention, as long as the modification of target activity when compared to the absence of the modulatory molecule can be assessed. Such a modulatory molecule can include small organic or inorganic molecules as well as large macromolecules. Specific examples of small molecules include KCl or BaCl₂. Examples of macromolecules which may be able to modulate the activity of the target of a cell include peptides, polypeptides, proteins, nucleic acid, carbohydrate and lipid. Functional or structural analogues or mimics of such compounds which exhibit substantially the same activation or inhibition activity are also included within the meaning of the term as used herein. The type, size or shape of the molecule is not important so long as the molecules can modulate the specific target activity of a cell.

The term “chemical library” or “array” refers to an intentionally created collection of differing molecules which can be prepared synthetically and screened for biological activity in a variety of different formats (e.g., libraries of soluble molecules, libraries of molecules bound to a solid support).

The term “target sequence” as used herein refers to a known DNA nucleotide sequence of a target wherein the DNA may be cDNA.

The term “target” as used herein refers to a protein of interest that has a known or suspected function or that has more than one known or suspected function. In this case, the term “function” refers to a signaling event, rather than a role in a disease state. Changes in the target's function or functional activity when exposed to potential modulator molecules are utilized to identify modulator molecules.

The term “target binding conditions” as used herein refers to environmental conditions that may effect the interaction between a target and a modulator molecule such as pH, temperature, and salt concentration.

The term “induction” or “induced” as used herein refers to the initiation of transcription and translation of the target sequence. Induction may occur in the presence of an inducer or in the absence of a repressor.

As used herein, the term “promoter” is a DNA sequence which extends upstream from the transcription initiation site and is involved in binding of RNA polymerase. The promoter may contain several short (<10 base pair) sequence elements that bind transcription factors, generally dispersed over >200 base pairs.

The term “inducible promoter” as used herein refers to a promoter that is transcriptionally active when bound to a regulator that activates transcription or when a regulator that represses transcription is absent. The inducible promoter is operatively linked to a target sequence.

The term “conditional expression” or “conditionally expresses” as used herein refers to the ability to activate and/or suppress the transcription of a target sequence by the presence or absence of an inducer molecule, an inducing condition or a regulator molecule.

The term “operably linked” as used herein refers to a DNA sequence and regulatory sequence(s) are connected in such a way as to permit gene expression when the appropriate molecules are bound to the regulatory sequences. When the inducible promoter is regulated by a repressor, gene expression may occur in the absence of a repressor. When the inducible promoter is regulated by an environmental condition, gene expression occurs by obtaining the inducing environmental condition (e.g. an increase in temperature activating a heat shock promoter).

The term “inducible cassette” as used herein refers to a sequence that may be inserted into a cloning vector that allows for the exogenous control of the transcription of a target sequence.

An “indicator molecule” refers to any molecule which allows visualization of the modulation of the target. For example, fluorescent indicator dyes which display altered fluorescence characteristics upon a change in membrane potential may be used.

The term “identify”, “identifying”, or “identification” as used herein refers to an act of assaying a compound or a plurality of compounds using the methods of the present invention to isolate a compound or compounds that modulate function or functional activity of a target.

The term “determine”, determining” or “determination” as used herein refers to the act of comparing assay measurements of a compound or compounds that may or may not have modulatory function or activity with a compound or compounds that do not have modulatory function or activity to isolate a compound or compounds that modulate a function or functional activity of a target.

As used herein, the term “physiological condition” refers to any biochemical or physiological change in the cell such that the event can be visualized using an indicator molecule according to the method of the present invention.

### DETAILED DESCRIPTION OF THE INVENTION

The present invention provides methods for identifying modulator molecules by screening these molecules against cells that conditionally express a target. In these methods cells that are clonally selected from populations stably transfected with an inducible vector construct may be controlled by the presence or absence of an exogenous cell-permeable inducer. This is especially advantageous when overexpression of the target interferes with the cell's growth or survival. Cells may be cultured in the absence of inducer to expand the population then transcription of the target sequence may be initiated for assay purposes. Assays to detect modulation may be different depending on the function of the target e.g. for a G-protein coupled receptor (“GPCR”) modulation may result in a change in cyclic AMP or intracellular calcium levels and modulation of an ion channel may result in a change in membrane potential. Moreover, the difference in functional activity of the target before and after induction provides an indication that the target is active and creates an ‘assay window’ that may be monitored during screening to verify that the cell is continuing to express the target throughout the testing period.

I. Inducible Vector Construct

The inducible vector construct provides control over the transcription of a target sequence such as an ion channel or GPCR by the presence or absence of an exogenous inducer or inducing condition. Therefore, expression may be increased or decreased to a level that when modulation occurs the user is able to distinguish between compounds that activate or inhibit a target's function or functional activity. In addition the detrimental effects associated with overexpression (e.g. toxicity and heterogeneous expression, e.g. variances in expression) of cells whether from the same population or of different type may be reduced. More specifically, the present invention provides methods for assaying transfected cells prior to induction (“steady state”) and after induction (“activated state”) of an inducible cassette. A measurement may also be taken once induction has ceased, and the transfected cells have returned to steady state. Steady state may be achieved by the absence of the inducer molecule or inducing condition or by the presence of a repressor such that the target sequence is unable to be transcribed. As previously described, current methods of modulator molecule discovery are unable to achieve conditions that allow for measurement of an initial steady state condition and an activated state condition. In addition, current methods are unable to monitor target activity during the course of a testing period.

The inducible vector construct may advantageously comprise an inducible cassette and a subcloning vector such as a plasmid or a cosmid. The inducible cassette regulates the expression of a target sequence positioned within the cassette by the induction of an inducible promoter positioned upstream of the target sequence. This induction occurs by adding an inducer molecule, removing a repressor, or changing an environmental condition that initiates transcription at the inducible promoter. Therefore, the user is able to exogenously “turn on” or “turn off” expression of the target sequence, and is advantageously also able to fine tune the level of expression.

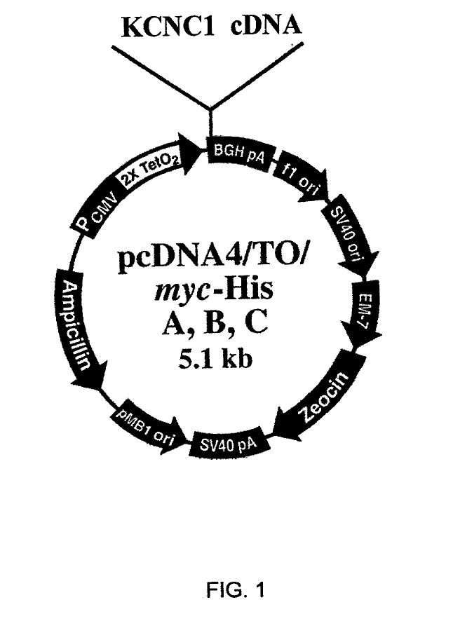

Some examples of inducible vector constructs that may be used are the tetracycline-dependent systems (Invitrogen, Carlsbad, Calif.; Clontech, Palo Alto Calif.) and the ecdysone inducible vector (Invitrogen, Carlsbad, Calif.). For example, the vector illustrated in FIG. 1 may be used for the present invention. The construct contains a region allowing regulated expression from a cytomegalovirus enhancer-promoter sequence containing two copies of the tet-O2 sequence, which is an enhancer that allows for highly regulated expression of the inserted gene. The vector additionally contains a gene conferring antibiotic (ampicillin) resistance, which is useful for bacterial subcloning procedures, and another gene conferring resistance to selection agents (such as zeocin) after transfection into the eukaryotic host cell. The construct of FIG. 1 also contains a multiple cloning site allowing for gene insertion downstream of the CMV tet-O2 promoter-enhancer sequence.

One embodiment of the inducible cassette comprises an inducible promoter, a selecting sequence, and a target insertion domain able to accept at least one target sequence. The inducible cassette may further comprise a reporter gene and/or at least one restriction site to enable ligation of the inducible cassette into a subcloning vector.

As an alternative to the use of the inducible cassette, an inducible promoter (and preferably also a gene providing for resistance to selection agents) can be inserted into the genome of a cell in which the target gene is endogenous. This would typically involve the use of 5′ and 3′ adapters enabling insertion of the inducible cassette into the host's genome by homologous recombination.

The inducible promoter provides exogenous control over the transcription of the target sequence by the presence or absence of an inducer molecule, a repressor, or an environmental condition that initiates transcription. A promoter may be selected based on a variety of characteristics such as its efficiency at initiating transcription, its ability to be exogenously controlled, the availability of its corresponding inducer and by the characteristics of the target.

The rate and efficiency of transcription by a given inducible promoter will vary depending on the promoter and its response to its corresponding inducer. Different inducible promoters are able to initiate transcription at different efficiencies and have different response curves to the absence or presence of their corresponding inducers. When the precise level of expression within the cell is to be quantitatively controlled a promoter with a rapid response to inducer may be desired (e.g. a minimal CMV promoter with two Tet-operator sequences 5′ of the promoter (as, for example, in the T-Rex system, Invitrogen, Carlsbad, Calif.). However, when precise control is not desired a promoter with basal activity may be utilized.

The availability of an inducer molecule may be regulated by biological accessibility or economic concerns. The ability for an inducer to be available biologically in an assay system may depend on its concentration, affinity and specificity. Correspondingly, the cost for obtaining a sufficient supply of inducer may be economically unfeasible. Tetracycline and its more stable analogue doxycycline are readily available inducers that may be utilized with the present invention. However, when the selecting sequence of the inducible cassette comprises a tetracycline resistance gene, a tetracycline inducible promoter may not be desired because the addition of the corresponding selecting media would also initiate transcription of the target sequence thereby reducing control over expression.

Cellular effects, such as for example cell growth or apoptosis, resulting from an expressed target may be a factor when choosing an inducible promoter. Steady state may be achieved when the promoter is “turned on” or “turned off” consequently promoters that are “turned on” in their steady state may be better suited for targets that do not interfere with cell survival or that inhibit deleterious effects such as for example apoptosis. Alternatively, promoters that are “turned off” in their steady state may be better suited for targets that interfere with cell growth, such as certain ion channels or apoptosis activators.

Some examples of promoters useful in the present invention are heat shock inducible promoter, metallothionin promoter, ecdysone-inducible promoter, FKBP dimerization inducible promoter, Gal4-estrogen receptor, fusion protein regulated promoter, Lac repressor, steroid inducible promoter, streptogramin responsive promoters, and tetracycline regulated promoters.

Selection is performed to select for cells that have been transfected with the inducible target construct. Mammalian cell transfection selection typically utilizes genes encoding resistance to selective agents such as, for example, zeocin, hygromycin, blasticidin, and geneticin.

The choice of a selecting sequence may depend on a variety of characteristics. The choice of a selecting sequence may depend on the ability to provide resistance to more than one selection agent. A selecting gene that confers resistance to a variety of selecting media may be desired to allow flexibility in the selecting procedure. Similarly, the addition of multiple selecting sequences may be combined into one cassette allowing the user to choose either for selection purposes.

The selecting sequence may be any sequence that allows selection of cells that express an inducible construct from those that do not following transfection. Selection may be conducted by addition of a selecting media that requires the expression of the selecting sequence for cell survival. Generally the selecting sequence may be an antibiotic resistance gene conferring resistance to its corresponding antibiotic or a gene that expresses a nutrient necessary for cell survival in a nutrient deficient culture media. Alternatively, single cells may be selected using fluorescent activated cell sorting (“FACS”) when the selecting sequence encodes a fluorescent protein such as, for example, a green fluorescent protein (“GFP”).

When choosing a selecting sequence for the inducible cassette it is preferable that the subcloning vector comprise a functionally different selecting sequence, so that the selection would not be specific to a construct comprising the inducible cassette. Correspondingly, when choosing a selecting sequence for the inducible cassette, it is preferable that the selecting sequence not provide resistance against an inducer.

One skilled in the art will recognize that when a cell is engineered to express different inducible cassettes, a different selection sequence may be inserted into each inducible cassette, allowing selection for cells able to express each. For example, zeocin resistance may be the selection sequence for one cassette, while hygromycin resistance may be the selection sequence for the second cassette. Therefore, when both are transfected into a cell, the appropriate media may contain zeocin and hygromycin. Some examples of selecting sequences useful in the present invention are genes that confer resistance to the selective agents zeocin, hygromycin and geneticin. Alternatively, nucleotide sequences that encode essential nutrients absent in nutrient deficient media may be utilized as selection sequences.

The target insertion domain is a sequence of nucleotides that enables ligation or insertion of a target sequence within the inducible cassette. The target insertion domain may comprise a single cloning site or a multiple cloning site (“MCS”) and may further comprise a reporter gene allowing detection of recombinant clones. Alternatively the target insertion domain may comprise thymidine overhangs enabling PCR products to be directly ligated to the cloning vector and may further comprise a reporter gene allowing detection of recombinant clones (Current Protocols in Molecular Biology, John Wiley Press).

In addition, a reporter gene may be positioned outside of the target insertion domain such that expression of the reporter occurs when the inducible cassette is expressed within the subcloning vector. In this configuration for example a luciferase reporter gene may be utilized to detect insertion of the inducible cassette into the subcloning vector. Other reporter genes that may be utilized with the present invention are b-galactosidase, chloramphenicol acetyltransferase and green fluorescent protein.

The inducible cassette may also comprise 5′ and 3′ insertion adapters enabling it to be inserted into the genome of the host organism by homologous recombination using standard recombination techniques (Mansour et al., Nature, 336:348-352,1988). In this configuration the insertion adapters are complementary to the non-coding region of the genome where the inducible cassette is to be inserted. Transcription of the target sequence may be controlled directly by the inducer or may be controlled through an intermediary whereby the inducer initiates transcription at an inducible promoter positioned within a second construct (“regulatory construct”) which may express a regulator. The regulator in this configuration controls the transcription of the target sequence.

The target sequence may be any nucleic acid sequence that encodes a cellular protein of pharmaceutical interest. The target sequence may be a known or a previously unidentified sequence. Known sequences may be selected by searching a database such as GenBank or SwissProt. Once the sequence of interest is selected primers may be designed such that the sequence may be amplified from a cDNA library (Current Protocols in Molecular Biology, John Wiley Press). Alternatively, the sequence may be purchased or obtained from a collection such as the I.M.A.G.E. Consortium [LLNL] cDNA Clones, (Lennon et al., Genomics 33:151-152, 1996). The cDNA clones provided by the I.M.A.G.E. Consortium are available through distributors including the ATCC (Rockville, Md.). The target sequence may encode a membrane-associated protein such as an ion channel protein, a receptor such as a G-protein coupled receptor target sequence, a nuclear hormone receptor target sequence, a cytokine receptor target sequence and a protein kinase-coupled receptor target sequence, a soluble protein such as an enzyme. A list of ion channel proteins that may be encoded by the target sequence of the present invention is listed in Table I, below.

TABLE I

| Name | Description of Ion Channel |
|---|---|
| ACCN1 | ACCN; amiloride-sensitive cation channel 1, |
| | Neuronal (degenerin); MDEG; BNC1; BnaC1; hBNaC1; Hs.6517 |
| ACCN2 | Amiloride-sensitive cation channel 2, neuronal; BNaC2; hBNaC2 |
| ACCN3 | TNAC1; ASIC3; amiloride-sensitive cation channel 3, testis |
| AQP1 | Aquaporin 1 (channel-forming integral protein, 28 kD); Hs.96074; |
| | CHIP28; Hs.74602 |
| BEC1 | Ether-a-go-go K(+) channel family member |
| BEC2 | Ether-a-go-go K(+) channel family member |
| CACC2 | Calcium-dependent chloride channel 2 |
| CACNA1A | CACNL1A4; EA2; MHP1; SCA6; calcium channel, voltage-dependent, |
| | P/Q type, alpha lA subunit; APCA; Acetazolamide responsive |
| | hereditary paroxysmal cerebellar ataxia; HPCA; familial periodic |
| | cerebellar ataxia/hereditary paroxysmal cerebellar ataxia/episodic |
| | ataxia; spinocerebellar ataxia 6; MHP; FHM; migraine, hemiplegic 1 |
| CACNA1B | CACNL1A5; CACNN; calcium channel, voltage-dependent, alpha 1B |
| | subunit, N type; calcium channel, N type |
| CACNA1C | CACNL1A1; calcium channel, voltage-dependent, L type, alpha 1C |
| | subunit; CCHL1A1 |
| CACNA1D | CACNL1A2; calcium channel, voltage-dependent, L type, alpha 1 D |
| | subunit; CCHL1A2 |
| CACNA1E | CACNL1A6; calcium channel, voltage-dependent, alpha 1E subunit |
| CACNA1F | Calcium channel, voltage-dependent, alpha 1F subunit; congenital |
| | stationary night blindness 2; CSNB2; CSNXB2 |
| CACNA1G | NBR13; calcium channel, voltage-dependent, T type, alpha-1G subunit |
| CACNA1H | Calcium channel, voltage-dependent, alpha 1H subunit |
| CACNA1I | Calcium channel, voltage-dependent, alpha 1I subunit |
| CACNA1S | CACNL1A3; MHS5; calcium channel, voltage-dependent, L type, alpha |
| | 1S subunit; malignant hyperthermia susceptibility 5; HypoPP; HOKPP; |
| | calcium channel, L type, alpha 1 polypeptide, isoform 3 (skeletal |
| | muscle, hypokalemic periodic paralysis) |
| CACNA2D1 | CACNA2; CACNL2A; MHS3; calcium channel, voltage-dependent, |
| | alpha 2/delta subunit; malignant hyperthermia susceptibility 3 |
| CACNA2D2 | CACNA2D; KIAA0558; calcium channel, voltage-dependent, alpha |
| | 2/delta subunit 2 |
| CACNB1 | CACNLB1; calcium channel,voltage-dependent, beta 1 subunit |
| CACNB2 | CACNLB2; MYSB; calcium channel, voltage-dependent, beta 2 subunit; |
| | myasthenic (Lambert-Eaton) syndrome antigen B |
| CACNB3 | CACNLB3; calcium channel,voltage-dependent, beta 3 subunit |
| CACNB4 | Calcium channel, voltage-dependent, beta 4 subunit |
| CACNG1 | CACNG; CACNLG; calcium channel,voltage-dependent, gamma |
| | subunit |
| CACNG2 | Calcium channel, voltage-dependent, gamma subunit 2 |
| CACNG3 | Calcium channel, voltage-dependent, gamma subunit 3 |
| CLCA1 | Chloride channel, calcium activated, 1; CaCC |
| CLCA2 | Chloride channel, calcium activated, 2 |
| CLCA3 | Chloride channel, calcium activated, family member 3 |
| CLCN1 | CLC1; chloride channel 1, skeletal muscle (Thomsen disease, autosomal |
| | dominant) |
| CLCN2 | Chloride channel 2; C1C-2 |
| CLCN3 | Chloride channel 3; C1C-3 |
| CLCN4 | Chloride channel 4; Hs.32790; C1C-4 |
| CLCN5 | NPHL2; chloride channel 5; Hs.3121; DENTS; nephrolithiasis 2 (X- |
| | linked, Dent disease) |
| CLCN6 | Chloride channel 6; C1C-6; KIAA0046 |
| CLCN7 | Chloride channel 7; C1C-7; CLC7 |
| CLCNKA | Chloride channel Ka; hC1C-Ka |
| CLCNKB | Chloride channel Kb; hC1C-Kb; Bartter syndrome, Type 3 |
| CLIC1 | Chloride intracellular channel 1; NCC27; p64CLCP |
| CLIC2 | Chloride intracellular channel 2 |
| CLIC3 | Chloride intracellular channel 3 |
| CLIC4 | Chloride intracellular channel 4; chloride intracellular channel 4 |
| | (mitochondrial); H1; huH1; mc3s5; p64H1; mtCLIC; CLIC4L |
| CLIC5 | Chloride intracellular channel 5 |
| CLIC6 | CLIC5; chloride intracellular channel 6; chloride intracellular channel 5; |
| | CLICL1 |
| CLNS1A | CLCI; chloride channel, nucleotide-sensitive, 1A; Icn |
| CLNS1B | Chloride channel, nucleotide-sensitive, 1B; Icn |
| CNGA1 | CNCG1; cyclic nucleotide gated channel alpha 1; CNG1; RCNC1; |
| | RCNCalpha; CNCG |
| CNGA2 | CNCA1; cyclic nucleotide gated channel alpha 2; CNG2; OCNC1; |
| | OCNCa; OCNCalpha; CNCA |
| CNGA3 | CNCG3; cyclic nucleotide gated channel alpha 3; CCNC1; CNG3; |
| | CCNCa; CCNCalpha |
| CNGB1 | CNCG2; CNCG3L; cyclic nucleotide gated channel beta 1; RCNC2; |
| | Hs.93909; GARP; GAR1; RCNCb; RCNCbeta; cyclic nucleotide gated |
| | channel (photoreceptor), cGMP gated 3 (gamma)-like |
| CNGB2 | CNCA2; cyclic nucleotide gated channel beta 2; OCNC2; OCNCbeta |
| CNGB3 | Cyclic nucleotide gated channel beta 3; ACHM3; achromatopsia-3; |
| | Pingelapese colorblindness |
| HCN1 | BCNG1; hyperpolarization activated cyclic nucleotide-gated potassium |
| | channel 1; brain cyclic nucleotide gated channel 1; HAC-2; BCNG-1 |
| HCN2 | BCNG2; hyperpolarization activated cyclic nucleotide-gated potassium |
| | channel 2; brain cyclic nucleotide gated channel 2; HAC-1; BCNG-2 |
| HCN4 | Hyperpolarization activated cyclic nucleotide-gated potassium channel 4 |
| KCNA1 | RBK1; HUK1; MBK1; AEMK; KV1.1; potassium voltage-gated |
| | channel, shaker-related subfamily, member 1 (episodic ataxia with |
| | myokymia) |
| KCNA10 | Potassium voltage-gated channel, shaker-related subfamily, member 10 |
| KCNA2 | Potassium voltage-gated channel, shaker-related subfamily, member 2; |
| | HK4; KV1.2 |
| KCNA3 | Hs.1750; MK3; HLK3; HPCN3; KV1.3; potassium voltage-gated |
| | channel, shaker-related subfamily, member 3 |
| KCNA4 | Hs.89647; Hs.1854; HK1; HPCN2; KV1.4; potassium voltage-gated |
| | channel, shaker-related subfamily, member 4 |
| KCNA4L | Potassium voltage-gated channel, shaker-related subfamily, member 4- |
| | like |
| KCNA5 | Hs.89509; HK2; HPCN1; KV1.5; potassium voltage-gated channel, |
| | shaker-related subfamily, member 5 |
| KCNA6 | Hs.2715; HBK2; KV1.6; potassium voltage-gated channel, shaker- |
| | related subfamily, member 6 |
| KCNA7 | HAK6; K( )1.7; potassium voltage-gated channel, shaker-related |
| | subfamily, member 7 |
| | KCNA1B; potassium voltage-gated channel, shaker-related subfamily, |
| | member 1 beta-1 subunit |
| KCNAB2 | KCNA2B; potassium voltage-gated channel, |
| | shaker-related subfamily, member 1 beta-2 subunit |
| KCNAB3 | KCNA3B; potassium voltage-gated channel, shaker-related subfamily, |
| | beta member 3 |
| KCNB1 | KV2.1; potassium voltage-gated channel, Shab-related subfamily, |
| | member 1 |
| KCNB2 | Potassium voltage-gated channel, Shab-related subfamily, member 2 |
| KCNC1 | KV3.1; potassium voltage-gated channel, Shaw-related subfamily, |
| | member 1 |
| KCNC2 | KV3.2; potassium voltage-gated channel, Shaw-related subfamily, |
| | member 2 |
| KCNC3 | K( )3.3; potassium voltage-gated channel, Shaw-related subfamily, |
| | member 3 |
| KCNC4 | KV3.4; HKSHIIIC; potassium voltage-gated channel, Shaw-related |
| | subfamily, member 4 |
| KCND1 | Potassium voltage-gated channel, Shal-related subfamily, member 1; |
| | KV4.1 |
| KCND2 | Potassium voltage-gated channel, Shal-related subfamily, member 2; |
| | RK5; KV4.2 |
| KCND3 | Potassium voltage-gated channel, Shal-related subfamily, member 3; |
| | KV4.3; KSHIVB |
| KCNE1 | Potassium voltage-gated channel, Isk-related family, member 1; minK; |
| | LQT5; ISK |
| KCNE2 | Potassium voltage-gated channel, Isk-related family, member 2; LQT5; |
| | LQT6; MiRP1 |
| KCNE3 | Potassium voltage-gated channel, Isk-related family, member 3; MIIRP2 |
| KCNF1 | KCNF; KV5.1; potassium voltage-gated channel, subfamily F |
| KCNG1 | KCNG; KV6.1; potassium voltage-gated channel, subfamily G |
| KCNH1 | Potassium voltage-gated channel, subfamily H, member 1 |
| KCNH2 | LQT2; long (electrocardiographic) QT syndrome 2; potassium voltage- |
| | gated channel, subfamily H, member 2; HERG; human ether-a-go-go- |
| | related gene |
| KCNJ1 | Potassium inwardly-rectifying channel, subfamily J, member 1; |
| | ROMK1; Kirl.1; Hs.463 |
| KCNJ10 | Potassium inwardly-rectifying channel, subfamily J, member 10; Kir4.1; |
| | Kirl.2; KCNJ13-PEN |
| KCNJ11 | Potassium inwardly-rectifying channel, subfamily J, member 11; BIR; |
| | Kir6.2 |
| KCNJ12 | Potassium inwardly-rectifying channel, subfamily J, member 12; Kir2.2 |
| KCNJ13 | Potassium inwardly-rectifying channel, subfamily J, member 13; Kirl.4; |
| | Kir7.1 |
| KCNJ14 | Potassium inwardly-rectifying channel, subfamily J, member 14; IRK4; |
| | Kir2.4 |
| KCNJ15 | Potassium inwardly-rectifying channel, subfamily J, member 15; Kir4.2; |
| | Kirl.3; KCNJ14-PEN |
| KCNJ16 | Potassium inwardly-rectifying channel, subfamily J, member 16; Kir5.1 |
| KCNJ2 | Potassium inwardly-rectifying channel, subfamily J, member 2; IRK1; |
| | Kir2.1; Hs.1547 |
| KCNJ3 | GIRK1; potassium inwardly-rectifying channel, subfamily J, member 3; |
| | Kir3.1 |
| KCNJ4 | Potassium inwardly-rectifying channel, subfamily J, member 4; HIR; |
| | HRK1; HIRK2; Kir2.3 |
| KCNJ5 | CIR; KATP1; potassium inwardly-rectifying channel, subfamily J, |
| | member 5; GIRK4; Kir3.4 |
| KCNJ6 | Potassium inwardly-rectifying channel, subfamily J, member 6; KCNJ7; |
| | GIRK2; KATP2; BIR1; Kir3.2; Hs.11173 |
| KCNJ8 | Potassium inwardly-rectifying channel, subfamily J, member 8; Kir6. 1 |
| KCNJ9 | Potassium inwardly-rectifying channel, subfamily J, member 9; G- |
| | protein coupled potassium inwardly-rectifying channel subfamily, |
| | member 3; GIRK3; Kir3.3 |
| KCNJN1 | Potassium inwardly-rectifying channel, subfamily J, inhibitor 1; Kir2.2v |
| KCNK1 | Potassium inwardly-rectifying channel, subfamily K, member 1; DPK; |
| | TWIK-1 |
| KCNK2 | Potassium inwardly-rectifying channel, subfamily K, member 2; TREK- |
| | 1 |
| KCNK3 | Potassium inwardly-rectifying channel, subfamily K, member 3; TASK |
| KCNK5 | TASK-2; potassium channel, subfamily K, member 5 (TASK-2) |
| KCNK6 | TOSS; TWIK-2; potassium channel, subfamily K, member 6 (TWIK-2) |
| KCNK7 | Potassium channel, subfamily K, member 7 |
| KCNMA1 | SLO; potassium large conductance calcium-activated channel, |
| | subfamily M, alpha member 1; Hs.62679 |
| KCNMB1 | Potassium large conductance calcium-activated channel, subfamily M, |
| | beta member 1; hslo-beta |
| KCNMB2 | Potassium large conductance calcium-activated channel, subfamily M, |
| | beta member 2 |
| KCNMB3 | KCNMBL; potassium large conductance calcium-activated channel, |
| | subfamily M, beta member 3 |
| KCNMB3L | KCNMBLP; potassium large conductance calcium-activated channel, |
| | subfamily M, beta member 3-like |
| KCNN1 | Potassium intermediate/small conductance calcium-activated channel, |
| | subfamily N, member 1; SK1; hSK1 |
| KCNN2 | Potassium intermediate/small conductance calcium-activated channel, |
| | subfamily N, member 2; hSK2 |
| KCNN3 | Potassium intermediate/small conductance calcium-activated channel, |
| | subfamily N, member 3; hSK3; SKCA3 |
| KCNN4 | Potassium intermediate/small conductance calcium-activated channel, |
| | subfamily N, member 4; hSK4; hKCa4; hIKCal |
| KCNQ1 | KCNA9; LQT1; KCNA8; potassium voltage-gated channel, KQT-like |
| | subfamily, member 1; LQTS; KVLQT1; LQT; long |
| | (electrocardiographic) QT syndrome, Ward-Romano syndrome 1 |
| KCNQ2 | Potassium voltage-gated channel, KQT-like subfamily, member 2 |
| KCNQ3 | Potassium voltage-gated channel, KQT-like subfamily, member 3 |
| KCNQ4 | DFNA2; potassium voltage-gated channel, KQT-like subfamily, |
| | member 4; deafhess, autosomal dominant 2 |
| KCNS1 | Potassium voltage-gated channel, delayed-rectifier, subfamily S, |
| | member 1; Kv9.1 |
| KCNS2 | Potassium voltage-gated channel, delayed-rectifier, subfamily S, |
| | member 2; Kv9.2 |
| KCNS3 | Potassium voltage-gated channel, delayed-rectifier, subfamily S, |
| | member 3; Kv9.3 |
| KVB3 | KVB3-LSB; potassium channel beta-subunit 3 |
| P2RX1 | Purinergic receptor P2X, ligand-gated ion channel, 1 |
| P2RX2 | P2X2; purinergic receptor P2X, ligand-gated ion channel, 2 |
| P2RX3 | Purinergic rece tor P2X, ligand-gated ion channel, 3; P2X3 |
| P2RX4 | Purinergic receptor P2X, ligand-gated ion channel, 4; P2X4 |
| P2RX5 | Purinergic receptor P2X, ligand-gated ion channel, 5; P2X5 |
| P2RX7 | Purinergic receptor P2X, ligand-gated ion channel, 7 |
| SCN10A | Sodium channel, voltage-gated, type X, alpha polypeptide |
| SCN11A | Sodium channel, voltage-gated, type XI, alpha polypeptide |
| SCN12A | Sodium channel, voltage-gated, type XII, alpha polypeptide |
| SCN1A | SCN1; sodium channel, voltage-gated, type I, alpha polypeptide |
| SCN1B | Hs.89634; sodium channel, voltage-gated, type I, beta polypeptide; |
| | Hs.1969 |
| SCN2A1 | SCN2A; HBSCI; sodium channel, voltage-gated, type II, alpha 1 |
| | polypeptide |
| SCN2A2 | HBSCII; sodium channel, voltage-gated, type II, alpha 2 polypeptide |
| SCN2B | Sodium channel, voltage-gated, type II, beta polypeptide |
| SCN3A | Sodium channel, voltage-gated, type III, alpha polypeptide |
| SCN4A | HYKPP; HYPP; hyperkalemic periodic paralysis (Gamstorp disease, |
| | adynamia episdica hereditaria); sodium channel, voltage-gated, type IV, |
| | alpha polypeptide |
| SCN4B | Sodium channel, voltage-gated, type IV, beta polypeptide |
| SCN5A | LQT3; sodium channel, voltage-gated, type V, alpha polypeptide (long |
| | (electrocardiographic) QT syndrome 3) |
| SCN6A | SCN7A; Hs.99945; sodium channel, voltage-gated, type VI, alpha |
| | polypeptide; sodium channel, voltage-gated, type VII, alpha polypeptide |
| SCN8A | MED; sodium channel, voltage-gated, type VIII, alpha polypeptide; |
| | motor endplate disease |
| SCN9A | Sodium channel, voltage-gated, type IX, alpha polypeptide |
| SCNN1A | SCNN1; sodium channel, nonvoltage-gated 1 alpha; EnaCa |
| SCNN1B | Sodium channel, nonvoltage-gated 1, beta (Liddle syndrome); EnaCb |
| SCNN1D | Sodium channel, nonvoltage-gated 1, delta; dNaCh; EnaCd |
| SCNN1G | Sodium channel, nonvoltage-gated 1, gamma; EnaCg |
| TRPC1 | Hs.78849; transient receptor potential channel 1 |
| TRPC2 | Transient receptor potential channel 2 |
| TRPC3 | Transient receptor potential channel 3 |
| TRPC4 | Transient receptor potential channel 4 |
| TRPC5 | Transient receptor potential channel 5 |
| TRPC6 | Transient receptor potential channel 6; TRP6 |
| TRPC7 | Transient receptor potential channel 7 |
| VDAC1 | Hs.2060; voltage-dependent anion channel 1 |
| VDAC1LP | Voltage-dependent anion channel 1-like pseudogene |
| VDAC1P | Voltage-dependent anion channel 1 pseudogene |
| VDAC2 | Voltage-dependent anion channel 2; Hs.78902 |
| VDAC3 | Voltage-dependent anion channel 3; HD-VDAC3; voltage-dependent |
| | anion channel 3 |
| VDAC4 | Voltage-dependent anion channel 4 |
| VDAC5P | VDAC3; voltage-dependent anion channel 3 |

Furthermore, the target sequence may encode an entire protein or merely an active portion of the protein. For example, the full length estrogen receptor or the isolated ligand binding domain of the same receptor may be used. A list of enzymes that may be encoded by the target sequence of the present invention is presented in Table II.

TABLE II

| Name | Description of Enzyme | |
|---|---|---|
| AACP | arylamide acetylase pseudogene; NATP | |
| AADAC | arylacetamide deacetylase (esterase); DAC | |
| AANAT | arylalkylamine N-acetyltransferase; SNAT | |
| AARS | alanyl-tRNA synthetase; Hs.75102 | |
| AATK | apoptosis-associated tyrosine kinase; AATYK; KIAA0641 | |
| ABAT | GABAT; 4-aminobutyrate aminotransferase | |
| ABCA4 | “ABCR; STGD1; ATP-binding cassette, sub-family A (ABC1), member | |
| | 4; ATP binding cassette transporter; retinitis pigmentosa 19 (autosomal | |
| | recessive); rim protein; FFM; STGD; ARMD2; Stargardt disease 1 | |
| | (fundus flavimaculatus, autosomal recessive)” | |
| ABCE1 | “RNS4I; RNASELI; ATP-binding cassette, sub-family E (OABP), | |
| | member 1; ribonuclease L (2′,5′-oligoisoadenylate synthetase-dependent) | |
| | inhibitor; OABP; RLI” | |
| ABCG1 | ATP-binding cassette, sub-family G (WHITE), member 1; WHITE1; | |
| | white (Drosophila) homolog 1, ATP binding casette transporter | |
| | superfamily; ABC8; WHITE” | |
| ABO | “ABO blood group (transferase A, alpha 1-3-N- | |
| | actylgalactosaminyltransferase; tansferase B, alpha 1-3, | |
| | galactosyltransferase); Hs.95985;ABO blood type” | |
| ABP1 | Hs.75741; AOC1; DAO; amiloride binding protein 1 (amine oxidase | |
| | (copper containing)) | |
| ACAA1 | ACAA; Hs.76260; acetyl-Coenzyme A acyltransferase (peroxisomal 3- | |
| | oxoacyl-Coenzyme A thiolase) | |
| ACAA2 | DSAEC; acetyl-Coenzyme A acyltransferase 2 (mitochondrial 3-oxoacyl- | |
| | Coenzyme A thiolase) | |
| ACACA | ACAC; acetyl-Coenzyme A carboxylase alpha; ACC | |
| ACACB | acetyl-Coenzyme A carboxylase beta; HACC275 | |
| ACAD | “acyl-Coenzyme A dehydrogenase, multiple” | |
| ACADL | “Hs.1209; acyl-Coenzyme A dehydrogenase, long chain” | |
| ACADM | “Hs.79158; MCAD; acyl-Coenzyme A dehydrogenase, C-4 to C-12 | |
| | straight chain” | |
| ACADS | “Hs.73966; acyl-Coenzyme A dehydrogenase, C-2 to C-3 short chain; | |
| | SCAD” | |
| ACADSB | “Hs.81934; acyl-Coenzyme A dehydrogenase, short/branched chain” | |
| ACADVL | “VLCAD; LCACD; acyl-Coenzyme A dehydrogenase, very long chain“ | |
| ACAT1 | Hs.37; T2; ACAT; THIL; acetyl-Coenzyme A acetyltransferase 1 | |
| | (acetoacetyl Coenzyme A thiolase) | |
| ACAT2 | acetyl-Coenzyme A acetyltransferase 2 (acetoacetyl Coenzyme A thiolase) | |
| ACE | DCP1; angiotensin I converting enzyme (peptidyl-dipeptidase A) 1; | |
| | dipeptidyl carboxypeptidase 1 (angiotensin I converting enzyme); DCP; | |
| | ACE1; Hs.76368; Hs.89639; Hs.99974 | |
| ACE2 | angiotensin I converting enzyme (peptidyl-dipeptidase A) 2 | |
| ACHAP | acetylcholinesterase-associated protein | |
| ACHE | acetylcholinesterase (YT blood group); Hs.89881; YT | |
| ACK | activated p21 cdc42Hs kinase | |
| ACLY | ATP citrate lyase | |
| ACO1 | “aconitase 1, soluble” | |
| ACO2 | “aconitase 2, mitochondrial; Hs.75900“ | |
| ACOX1 | “ACOX; acyl-Coenzyme A oxidase 1, palmitoyl; acyl-Coenzyme A | |
| | oxidase; PALMCOX” | |
| ACOX2 | “acyl-Coenzyme A oxidase 2, branched chain; BRCACOX; branched- | |
| | chain acyl-CoA oxidase, peroxisomal; BRCOX” | |
| ACOX3 | “acyl-Coenzyme A oxidase 3, pristanoyl” | |
| ACP1 | “Hs.75393; acid phosphatase 1, soluble” | |
| ACP2 | “Hs.75589; acid phosphatase 2, lysosomal” | |
| ACP5 | “Hs.89806; acid phosphatase 5, tartrate resistant; Hs.1211” | |
| ACPP | “Hs.1852; acid hosphatase, rostate" | |
| ACVR1 | “ACVRLK2; activin A receptor, type I; SKR1; ALK2; activin A | |
| | receptor, type II-like kinase 2” | |
| ACY1 | Hs.79; aminoacylase 1 | |
| ACY1L | AN; 91184800; aminoacylase 1-like | |
| ACYP1 | “acylphosphatase 1, erythrocyte (common) type; ACYPE” | |
| ACYP2 | “acylphosphatase 2, muscle type” | |
| AD2 | “Alzheimer disease 2 (APOEE4-associated, late onset)” | |
| AD5 | Alzheimer disease 5; AD5-PEN | |
| ADA | Hs.1217; adenosine deaminase | |
| ADAM1 | FTNAP; PH-30A; a disintegrin and metalloproteinase domain 1 (fertilin | |
| | alpha) | |
| ADAM10 | a disintegrin and metalloprotease domain 10; kuz | |
| ADAM11 | “MDC; metalloproteinase-like, disintegrin-like, cysteine-rich protein” | |
| ADAM12 | a disintegrin and metalloproteinase domain 12 (meltrin alpha); MLTN; | |
| | MCMP; Mltna | |
| ADAM13 | a disintegrin and metalloproteinase domain 13 | |
| ADAM14 | ADM-1; a disintegrin and metalloproteinase domain 14 | |
| ADAM15 | a disintegrin and metalloproteinase domain 15 (metargidin); MDC15 | |
| ADAM16 | MDC16; a disintegrin and metalloproteinase domain 16 | |
| ADAM18 | ADAM27; TMDCIII; a disintegrin and metalloproteinase domain 18 | |
| ADAM19 | MLTNB; a disintegrin and metalloproteinase domain 19 (meltrin beta) | |
| ADAM20 | a disintegrin and metalloproteinase domain 20 | |
| ADAM21 | a disintegrin and metalloproteinase domain 21 | |
| ADAM22 | a disintegrin and metalloproteinase domain 22; MDC2 | |
| ADAM23 | a disintegrin and metalloproteinase domain 23; MDC-L; MDC3 | |
| ADAM24 | a disintegrin and metalloproteinase domain 24 | |
| ADAM25 | a disintegrin and metalloproteinase domain 25 | |
| ADAM26 | a disintegrin and metalloproteinase domain 26 | |
| ADAM28 | a disintegrin and metalloproteinase domain 28 | |
| ADAM29 | a disintegrin and metalloproteinase domain 29; svph1 | |
| ADAM30 | a disintegrin and metalloproteinase domain 30; svph4 | |
| ADAM3B | CYRN2; cyritestin 2; a disintegrin and metalloproteinase domain 3b | |
| | (cyritestin 2) | |
| ADAM4 | TMDCV; a disintegrin and metalloproteinase domain 4 | |
| ADAM5 | TMDCII; a disintegrin and metalloproteinase domain 5 | |
| ADAM6 | TMDCIV; a disintegrin and metalloproteinase domain 6 | |
| ADAM7 | EAPI; GP-83; a disintegrin and metalloproteinase domain 7 | |
| ADAM8 | a disintegrin and metalloprotease domain 8 | |
| ADAM9 | a disintegrin and metalloproteinase domain 9 (meltrin gamma); MCMP; | |
| | MCMP-PEN; ADAM12; myeloma cell metalloproteinase | |
| ADAMTS1 | “a disintegrin-like and metalloprotease (reprolysin type) with | |
| | thrombospondin type 1 motif, 1; METH1; METH-1” | |
| ADAMTS2 | “a disintegrin-like and metalloprotease (reprolysm type) with | |
| | thrombospondin type 1 motif, 2; PCINP; hPCPNI; ADAM-TS2; | |
| | ADAMTS-3; EDS VIIC; EDS VIIB” | |
| ADAMTS4 | “a disintegrin-like and metalloprotease (reprolysin type) with | |
| | thrombospondin type 1 motif, 4; ADMP-1; ADAMTS-2” | |
| ADAMTS5 | “a disintegrin-like and metalloprotease (reprolysin type) with | |
| | thrombospondin type 1 motif, 5 (aggrecanase-2); ADMP-2; ADAMTS11” | |
| ADAMTS6 | “ADAM-TS6; a disintegrin-like and metalloprotease (reprolysin type) | |
| | with thrombospondin type 1 motif, 6” | |
| ADAMTS7 | “ADAM-TS7; a disintegrin-like and metalloprotease (reprolysin type) | |
| | with thrombospondin type 1 motif, 7” | |
| ADAMTS8 | “METH2; a disintegrin-like and metalloprotease (reprolysin type) with | |
| | thrombospondin type 1 motif, 8” | |
| ADAR | “Hs.7957; adenosine deaminase, RNA-specific” | |
| ADARB1 | “adenosine deaminase, RNA-specific, B1 (homolog of rat RED1); | |
| | ADAR2” | |
| ADARB2 | “adenosine deaminase, RNA-specific, B2 (homolog of rat BLUE); | |
| | RED2; hRED2” | |
| ADAT1 | “adenosine deaminase, tRNA-specific 1; hADAT1” | |
| ADCP1 | adenosine deaminase complexing protein 1 | |
| ADCY1 | Hs.139; adenylate cyclase 1 (brain) | |
| ADCY2 | HBAC2; adenylate cyclase 2 (brain) | |
| ADCY3 | adenylate cyclase 3 | |
| ADCY4 | adenylate cyclase 4 | |
| ADCY5 | adenylate cyclase 5 | |
| ADCY6 | adenylate cyclase 6 | |
| ADCY7 | KIAA0037; adenylate cyclase 7 | |
| ADCY8 | Hs.2522; ADCY3; HBAC1; adenylate cyclase 8 (brain) | |
| ADCY9 | adenylate cyclase 9 | |
| ADGYAP1 | Hs.68137; PACAP; adenylate cyclase activating polypeptide 1 (pituitary) | |
| ADCYAP1R1 | PACAPR; adenylate cyclase activating polypeptide 1 (pituitary) receptor | |
| | type 1 | |
| ADE2C1 | ade2 (S .cerevisiae) complementing; Multifunctional SAICAR | |
| | synthetase/AIR carboxylase | |
| ADE2H1 | multifunctional polypeptide similar to SAICAR synthetase and AIR | |
| | carboxylase | |
| ADH1 | “Hs.73843; alcohol dehydrogenase 1 (class I), alpha polypeptide” | |
| ADH2 | “Hs.4; alcohol dehydrogenase 2 (class 1), beta polypeptide” | |
| ADH3 | “Hs.2523; alcohol dehydrogenase 3 (class I), gamma polypeptide” | |
| ADH4 | “Hs.1219; alcohol dehydrogenase 4 (class II, pi polypetide” | |
| ADH5 | “Hs.78989; alcohol dehydrogenase 5 (class III, chi polypeptide” | |
| ADH5P1 | “alcohol dehydrogenase 5 (class III), chi polypeptide, pseudogene 1“ | |
| ADH6 | alcohol dehydrogenase 6 (class V) | |
| ADH7 | “alcohol dehydrogenase 7 (class IV), mu or sigma polypetide; Hs.389” | |
| ADK | Hs.94382; adenosine kinase | |
| ADPRH | ADP-ribosylarginine hydrolase | |
| ADPRT | ADP-ribosyltransferase (NAD+; poly (ADP-ribose) polymerase); PARP; | |
| | Hs.76105; PPOL | |
| ADPRTL1 | ADP-ribosyltransferase (NAD+; poly (ADP-ribose) polymerase)-like 1; | |
| | PH5P; PARPL; VPARP; KIAA0177 | |
| ADPRTL2 | ADP-ribosyltransferase (NAD+; poly(ADP-ribose) polymerase)-like 2; | |
| | Adprt2; PARP-2 | |
| ADPRTL3 | ADP-ribosyltransferase (NAD+; poly (ADP-ribose) polymerase)-like 3; | |
| | PARP-2 | |
| ADPRTP1 | PPOLP1; ADP-ribosyltransferase (NAD+; poly (ADP-ribose) | |
| | polymerase) pseudogene 1 | |
| ADPRTP2 | PPOLP2; ADP-ribosyltransferase (NAD+; poly (ADP-ribose) | |
| | polymerase) pseudogene 2 | |
| ADRBK1 | “Hs.83636; GRK2; BARK1; adrenergic, beta, receptor kinase 1” | |
| ADRBK2 | “GRK3; BARK2; adrenergic, beta, receptor kinase 2" | |
| ADSL | adenylosuccinate lyase; adenylosuccinase | |
| ADSS | adenylosuccinate synthase | |
| AFG3L1 | “AFG3 (ATPase family gene 3, yeast)-like 1; AFG3” | |
| AFG3L2 | “AFG3 (ATPase family gene 3, yeast)-like 2” | |
| AGA | Hs.21488; aspartylglucosaminidase | |
| AGL | “Hs.904; amylo-1,6-glucosidase, 4-alpha-glucanotransferase (glycogen | |
| | debranching enzyme, glycogen storage disease type III)” | |
| AGPAT1 | “1-acylglycerol-3-phosphate O-acyltransferase 1 (lysophosphatidic acid | |
| | acyltransferase, alpha); LPAAT-ALPHA; G15; lysophosphatidic acid | |
| | acyltransferase alpha” | |
| AGPS | alkylglycerone phosphate synthase; ADHAP; ADHAP-PEN; alkyl- | |
| | dihydroxyacetonephosphate; ADAS; ADPS; ADHAPS; ADAP-S; | |
| | ALDHPSY | |
| AGXT | SPAT; Hs.81554; alanine-glyoxylate aminotransferase (oxalosis I; | |
| | hyperoxaluria I; glycolicaciduria; serine-pyruvate aminotransferase) | |
| AHCY | Hs.85111; 5-adenosylhomocysteine hydrolase | |
| AHCYL1 | XPVKONA; 5-adenosylhomocysteine hydrolase-like 1 | |
| AHHR | AHH; aryl hydrocarbon hydroxylase regulator | |
| AIED | “OA2; Aland island eye disease (Forsius-Eriksson ocular albinism, ocu- | |
| | lar albinism type 2)” | |
| AK1 | adenylate kinase 1 | |
| AK2 | adenylate kinase 2 | |
| AK3 | adenylate kinase 3 | |
| AK3P1 | adenylate kinase 3 pseudogene 1 | |
| AKAP1 | “AKAP84; AKAP84-PEN; A kinase anchor protein(spermatid, p84)” | |
| AKAP10 | D-AKAP2; AKAP10-PENDING; A kinase (PRKA) anchor protein 10 | |
| AKAP11 | A kinase (PRKA) anchor protein 11; AKAP220; KIAA0629 | |
| AKAP13 | BRX; HT31; AKAP13-PENDING; A kinase (PRKA) anchor protein 13 | |
| AKAP2 | AKAP-KL; KIAA0920; AKAP2-PENDING; DKFZP564L0716; A kinase | |
| | (PRKA) anchor protein 2 | |
| AKAP3 | SOB1; AKAP110; AKAP3-PENDING; A kinase (PRKA) anchor protein | |
| | 3 | |
| AKAP4 | P82; FSC1; AKAP82; HAKAP82; AKAP4-PENDING; A kinase | |
| | (PRKA) anchor protein 4 | |
| AKAP5 | AKAP75; AKAP79; AKAPS-PENDING; A kinase (PRKA) anchor | |
| | protein 5 | |
| AKAP7 | AKAP18; AKAP7-PENDING; A kinase (PRKA) anchor protein 7 | |
| AKAP8 | AKAP95; AKAP8-PENDING; DKFZP586B1222; A kinase (PRKA) | |
| | anchor protein 8 | |
| AKAP9 | YOTIAO; CG-NAP; AKAP450; AKAP350; AKAP120; KIAA0803; A | |
| | kinase (PRKA) anchor protein 9 | |
| AKR1A1 | “aldo-keto reductase family 1, member Al (aldehyde reductase); ALR” | |
| AKR1B1 | “ALDR1; aldo-keto reductase family 1, member B1 (aldose reductase); | |
| | aldehyde reductase 1 (low Km aldose reductase); Hs.75313; AR” | |
| AKR1C1 | “DDH1; dihydrodiol dehydrogenase 1 (trans-1,2-dihydrobenzene-1,2-diol | |
| | dehydrogenase, high affinity bile acid binding); Hs.78183; DDH; MBAB” | |
| ARR1C2 | “DDH2; dihydrodiol dehydrogenase 2 (trans-1,2-dihydrobenzene- 1,2- | |
| | diol dehydrogenase)” | |
| AKR1C3 | “aldo-keto reductase family 1, member C3 (3-alpha hydroxysteroid | |
| | dehydrogenase, type II); KIAA0119” | |
| AKR1C4 | CHDR; chlordecone reductase; Hs.76790 | |
| AKR1D1 | “SRD5B1; aldo-keto reductase family 1, member D1 (delta 4-3- | |
| | ketosteroid-5-beta-reductase); steroid-5-beta-reductase, beta polypeptide | |
| | 1 (3-oxo-5 beta-steroid delta 4-dehydrogenase beta 1)” | |
| AKR7A2 | “aldo-keto reductase family 7, member A2 (aflatoxin aldehyde re- | |
| | ductase); AFAR; AKR7” | |
| AKR7A3 | “aldo-keto reductase family 7, member A3 (aflatoxin aldehyde reductase)” | |
| AKT3 | “v-akt murine thymoma viral oncogene homolog 3 (protein kinase B, | |
| | gamma); protein kinase B gamma; PKBG; PRKBG; RAC-gamma” | |
| ALAD | “aminolevulinate, delta-, dehydratase” | |
| ALAS1 | “Hs.78712; ALAS; aminolevulinate, delta-, synthase 1; Hs.2530” | |
| ALAS2 | “Hs.79103; ASB; aminolevulinate, delta-, synthase 2 | |
| | (sideroblastic/hypochromic anemia)” | |
| ALDH1 | “aldehyde dehydrogenase 1, soluble; Hs.76392; PUMB1” | |
| ALDH10 | SLS; aldehyde dehydrogenase 10 (fatty aldehyde dehydrogenase); | |
| | Sjogren-Larsson syndrome; FALDH | |
| ALDH2 | “Hs.74630; aldehyde dehydrogenase 2, mitochondrial” | |
| ALDH3 | Hs.575; aldehyde dehydrogenase 3 | |
| ALDH4 | aldehyde dehydrogenase 4 (glutamate gamina-semialdehyde | |
| | dehydrogenase; pyrroline-5-carboxylate dehydrogenase); P5CDh | |
| ALDH5 | ALDHX; aldehyde dehydrogenase 5 | |
| ALDH5A1 | SSADH; NAD+-dependent succinic semialdehyde dehydrogenase; SSDH | |
| ALDH6 | Hs.75746; aldehyde dehydrogenase 6 | |
| ALDH7 | aldehyde dehydrogenase 7 (NOTE: redefinition of symbol); Hs.3116; | |
| | ALDHA; Hs.2533 | |
| ALDH8 | aldehyde dehydroenase 8; Hs.87539 | |
| ALDH9 | “aldehyde dehydrogenase 9 (gamma-aminobutyraldehyde dehydrogenase, | |
| | E3 isozyme)” | |
| ALDOA | “HS.75181; aldolase A, fructose-bisphosphate” | |
| ALDOAP1 | “aldolase A, fructose-bisphosphate pseudogene 1” | |
| ALDOAP2 | “aldolase A, fructose-bisphosphate pseudogene 2” | |
| ALDOB | “aldolase B, fructose-bisphosphate; Hs.75592; ALDO2” | |
| ALDOC | “aldolase C, fructose-bisphosphate” | |
| ALDRL1 | aldehyde reductase (aldose reductase)-like 1 | |
| ALDRL2 | aldehyde reductase (aldose reductase)-like 2 | |
| ALDRL3 | aldehyde reductase (aldose reductase)-like 3 | |
| ALDRL4 | aldehyde reductase (aldose reductase)-like 4 | |
| ALDRP | aldehyde reductase (aldose reductase) pseudogene | |
| ALK | anaplastic lymphoma kinase (Ki-1) | |
| ALOX12 | arachidonate 12-lipoxygenase; Hs.1200 | |
| ALOX12B | “arachidonate 12-lipoxygenase, 12R type” | |
| ALOX12P1 | ALOX12P; arachidonate 12-lipoxygenase pseudogene 1 | |
| ALOX12P2 | arachidonate 12-lipoxygenase pseudogene 2 | |
| ALOX15 | arachidonate 15-lipoxygenase; Hs.73809 | |
| ALOX15B | “arachidonate 15-lipoxygenase, second type” | |
| ALOX5 | arachidonate 5-lipoxygenase; Hs.89499 | |
| ALOX5AP | arachidonate 5-lipoxygenase-activating protein; FLAP | |
| ALPI | “alkaline phoshatase, intestinal” | |
| ALPL | “alkaline phosphatase, liver/bone/kidney; Hs.2241; HOPS; TNSALP; | |
| | tissue-nonspecific ALP” | |
| ALPP | “Hs.73847; alkaline phosphatase, placental (Regan isozyme)” | |
| ALPPL2 | “alkaline phoshatase, placental-like 2” | |
| AMDI | Hs.75744; S-adenosylmethionine decarboxylase 1 | |
| AMD2 | S-adenosylmethionine decarboxylase 2 (pseudogene); AMD; S- | |
| | adenosylmethionine decarboxylase 2 | |
| AMPD1 | adenosine monophosphate deaminase 1 (isoform M) | |
| AMPD2 | adenosine monophosphate deaminase 2 (isoform L) | |
| AMPD3 | Hs.83918; adenosine monophosphate deaminase 3 (isoform E) | |
| AMT | Hs.102; aminomethyltransferase (glycine cleavage system protein T) | |
| AMY1A | “AMY1; amylase, alpha 1A; salivary” | |
| AMY1B | “AMY1; amylase, alpha 1B; salivary” | |
| AMY1C | “AMY1; amylase, alpha 1C; salivary” | |
| AMY2A | “AMY2; amylase, alpha 2A; pancreatic” | |
| AMY2B | “AMY2; amylase, alpha 2B; pancreatic” | |
| AMYP1 | “AMY2P; amylase, alpha pseudogene 1” | |
| ANG | “angiogenin, ribonuclease, RNase A family, 5; RNASE5” | |
| ANPEP | “Hs.1239; PEPN; CD13; alanyl (membrane) aminopeptidase | |
| | (aminopeptidase N, aminopeptidase M, microsomal aminopeptidase, | |
| | CD13, p150)” | |
| ANXA2 | “ANX2; CAL1H; arylsulfatase B; Hs.74470; LTP2; LPC2D; ANX2L4; | |
| | annexin II (lipocortin II); calpactin I, heavy polypeptide (p36)” | |
| ANXA3 | “ANX3; Hs.1378; annexin III (lipocortin III, 1,2-cyclic-inositol-phosphate | |
| | phosphodiesterase, placental anticoagulant protein III, calcimedin 35- | |
| | alpha)” | |
| AOAH | Hs.82542; acyloxyacyl hydrolase (neutrophil) | |
| AOC2 | “amine oxidase, copper containing 2 (retina-specific); RAO; DAO2” | |
| AOC3 | “VAP-1; amine oxidase, copper containing 3 (vascular adhesion protein | |
| | 1)” | |
| AOE372 | thioredoxin peroxidase (antioxidant enzyme) | |
| AOX1 | aldehyde oxidase 1; AO | |
| APAA | N-acetylglucosamine-1-phosphodiester alpha-N-acetylglucosaminidase; | |
| | LOC51172 | |
| APAF1 | apoptotic protease activating factor 1; CED4 | |
| APC10 | DOC1; anaphase-promoting complex 10 | |
| APEH | D3S48E; Hs.78223; N-aclaminoacyl-peptide hydrolase | |
| APEX | APE; APEX nuclease (multifunctional DNA repair enzyme); REF1; | |
| | HAP1; apurinic/apyrimidinic (abasic) endonuclease | |
| APP | “amyloid beta (A4) precursor protein (protease nexin-II, Alzheimer | |
| | disease); Hs.74600; AD1” | |
| APRT | adenine phosphoribosyltransferase | |
| APT6M8-9 | “ATPase, H+ transporting, lysosomal (vacuolar proton pump) membrane | |
| | sector associated protein M8-9” | |
| AR | androgen receptor (dihydrotestosterone receptor; testicular feminization; | |
| | spinal and bulbar muscular atrophy; Kennedy disease); Hs.99915; DHTR; | |
| | SBMA; AIS; NR3C4; Hs.1241 | |
| ARD1 | “TE2; N-acetyltransferase, homolog of S. cerevisiae ARD1” | |
| ARG1 | “Hs.77600; arginase, liver” | |
| ARG2 | “arginase, type II; Hs.79338” | |
| ARHGAP1 | Rho GTPase activating protein 1; RhoGAP; p50rhoGAP | |
| ARHGAP4 | Rho GTPase activating protein 4; KIAA0131; C1; p115; RhoGAP4 | |
| ARHGAP5 | Rho GTPase activating protein 5; p190-B; RhoGAP5 | |
| ARHGAP6 | Rho GTPase activating protein 6; rhoGAPX-1 | |
| ARSA | Hs.88251; arylsulfatase A | |
| ARSB | arylsulfatase B; Hs.1256 | |
| ARSC2 | “ARSC; arylsulfatase C, isozyme F” | |
| ARSD | arylsulfatase D; Hs. 1256 | |
| ARSDP | arysfulfatase D pseudogene | |
| ARSE | CDPX; CDPX1; arylsulfatase E (chondrodysplasia punctata 1) | |
| ARSEP | arysulfatase E pseudogene | |
| ARSF | arysulfatase F | |
| ART1 | ADP-ribosyltransferase 1; ART2 | |
| ART2P | RT6; ADP-ribosyltransferase 2 pseudogene (RT6 antigen (rat) homolog); | |
| | ART1P | |
| ART3 | ADP-ribosyltransferase 3; ADP-ribosyltransferase 3 | |
| ART4 | ADP-ribosyltransferase 4 | |
| ASAH | N-acylsphingosine amidohydrolase; AC | |
| ASK | activator of S phase kinase | |
| ASL | Hs.61258; argininosuccinate lyase | |
| ASLL | argininosuccinate lyase-like | |
| ASM3A | acid sphingomyelinase-like phosphodiesterase | |
| ASMT | acetylserotonin O-methyltransferase; HIOMT | |
| ASMTL | acetylserotonin N-methyltransferase-like | |
| ASNS | asparagine synthetase | |
| ASNSL1 | asparagine synthetase-like 1 | |
| ASNSL2 | asparagine synthetase-like 2 | |
| ASPA | “aspartoacylase (aminoacylase 2, Canavan disease); Hs.32042; ASP” | |
| ASPH | aspartate beta-hydroxylase | |
| ASS | “argininosuccinate synthetase; Hs.76753; ASS1; CTLN1; citrullinemia, | |
| | classic” | |
| ASSP1 | argininosuccinate synthetase pseudogene 1 | |
| ASSP10 | argininosuccinate synthetase pseudogene 10 | |
| ASSP11 | argininosuccinate synthetase pseudogene 11 | |
| ASSP12 | argininosuccinate synthetase pseudogene 12 | |
| ASSP13 | argininosuccinate synthetase pseudogene 13 | |
| ASSP14 | argininosuccinate synthetase pseudogene 14 | |
| ASSP2 | argininosuccinate synthetase pseudogene 2 | |
| ASSP3 | argininosuccinate synthetase pseudogene 3 | |
| ASSP4 | argininosuccinate synthetase pseudogene 4 | |
| ASSP5 | argininosuccinate synthetase pseudogene 5 | |
| ASSP6 | argininosuccinate synthetase pseudogene 6 | |
| ASSP7 | argininosuccinate synthetase pseudogene 7 | |
| ASSP8 | argininosuccinate synthetase pseudogene 8 | |
| ASSP9 | argininosuccinate synthetase pseudogene 9 | |
| ATE1 | arginyltransferase 1 | |
| ATIC | 5-aminoimidazole-4-carboxamide ribonucleotide formyltransferase/IMP | |
| | cyclohydrolase; PURH; AICARFT/IMPCHASE | |
| ATP-BL | “ATP synthase, subunit b-like” | |
| ATP1A1 | “ATPase, Na+/K+ transporting, alpha 1 polypeptide” | |
| ATP1A2 | “ATPase, Na+/K+ transporting, alpha 2 (+) polypetide” | |
| ATP1A3 | “ATPase, Na+/K+ transporting, alpha 3 polypeptide” | |
| ATP1AL1 | “Hs.1165; ATPase, Na+/K+ transporting, alpha polypeptide-like 1” | |
| ATP1AL2 | “ATPase, Na+/K+ transporting, alpha polypeptide-like 2; ATP1A4” | |
| ATP1B1 | “ATPase, Na+/K+ transporting, beta 1 polypeptide; Hs.78629; ATP1B” | |
| ATP1B2 | “ATPase, Na+/K+ transporting, beta 2 polypeptide; Hs.90792; AMOG; | |
| | Hs.78854” | |
| ATP1B3 | “ATPase, Na+/K+ transporting, beta 3 polypeptide” | |
| ATP1B3P1 | “ATPase, Na+/K+ transporting, beta 3 pseudogene 1” | |
| ATP1B4 | “B4 ATPase, (Na+)/K+ transporting, beta 4 polypeptide; X,K- | |
| | ATPase beta-m subunit” | |
| ATP1BL1 | “ATPase, Na+/K+ transporting, beta polypeptide-like 1” | |
| ATP1G1 | “ATPase, Na+/K+ transporting, gamma 1 polypeptide” | |
| ATP2A1 | “ATP2A; SERCA1; ATPase, Ca++ transporting, cardiac muscle, fast | |
| | twitch 1” | |
| ATP2A2 | “DAR; Darier disease (keratosis follicularis); ATPase, Ca++ transporting, | |
| | cardiac muscle, slow twitch 2; Hs.1526; ATP2B; SERCA2” | |
| ATP2A3 | “ATPase, Ca++ transporting, ubiquitous” | |
| ATP2B1 | “PMCA1; ATPase, Ca++ transporting, plasma membrane 1” | |
| ATP2B2 | “ATPase, Ca++ transporting, plasma membrane 2 (NOTE: redefinition of | |
| | symbol); Hs.89512; PMCA2” | |
| ATP2B3 | “Hs.2009; PMCA3; ATPase, Ca++ transporting, plasma membrane 3” | |
| ATP2B4 | “Hs.995; PMCA4; ATP2B2; ATPase, Ca++ transporting, plasma | |
| | membrane 4” | |
| ATP3 | “ATPase, Mg++ transporting” | |
| ATP4A | “ATP6A; ATPase, H+/K+ transporting, alpha polypeptide” | |
| ATP4B | “ATP6B; ATPase, H+/K+ transporting, beta polypeptide” | |
| ATP5 | “ATP synthase, H+ transporting, mitochondrial; Hs.73851; ATPM; | |
| | ATP5A” | |
| ATP5A1 | “ATP5A; ATP synthase, H+ transporting, mitochondrial F1 complex, | |
| | alpha subunit, isoform 1, cardiac muscle; Hs.1182; OMR; ATPM” | |
| ATP5A2 | “ATP synthase, H+ transporting, mitochondrial F1 complex, alpha | |
| | subunit, isoform 2, non-cardiac muscle” | |
| ATP5AL1 | “ATP synthase, H+ transporting, mitochondrial F1 complex, alpha | |
| | subunit, isoform 1, cardiac muscle-like 1” | |
| ATP5AL2 | “ATP synthase, H+ transporting, mitochondrial F1 complex, alpha | |
| | subunit, isoform 2, non-cardiac muscle-like 2” | |
| ATP5AP1 | “ATP synthase, H+ transporting, mitochondnal F1 complex, alpha | |
| | subunit, pseudogene 1” | |
| ATP5AP2 | “ATP synthase, H+ transporting, mitochondrial F1 complex, alpha | |
| | subunit, pseudogene 2” | |
| ATP5AP3 | “ATP synthase, H+ transporting, mitochondrial F1 complex, alpha | |
| | subunit, pseudogene 3” | |
| ATP5B | “Hs.25; ATPSB; ATP synthase, H+ transporting, mitochondrial F1 | |
| | complex, beta polypeptide” | |
| ATP5BL1 | “ATPSBL1; ATP synthase, H+ transporting, mitochondrial F1 complex, | |
| | beta polypeptide-like 1” | |
| ATP5BL2 | “ATPSBL2; ATP synthase, H+ transporting, mitochondrial F1 complex, | |
| | beta polypeptide-like 2” | |
| ATP5C1 | “ATP5C; ATP synthase, H+ transporting, mitochondrial F1 complex, | |
| | gamma polypeptide 1” | |
| ATP5C2 | “ATP synthase, H+ transporting, mitochondrial F1 complex, gamma | |
| | polypeptide 2” | |
| ATP5CL1 | “ATP synthase, H+ transporting, mitochondrial F1 complex, gamma | |
| | polypeptide-like 1” | |
| ATP5CL2 | “ATP synthase, H+ transporting, mitochondrial F1 complex, gamma | |
| | polypeptide-like 2” | |
| ATP5D | “Hs.89761; ATP synthase, H+ transporting, mitochondrial F1 complex, | |
| | delta subunit” | |
| ATP5E | “ATP synthase, H+ transporting, mitochondrial F1 complex, epsilon | |
| | subunit” | |
| ATP5EP1 | “ATP synthase, H+ transporting, mitochondrial F1 complex, epsilon | |
| | subunit pseudogene 1” | |
| ATP5F1 | “Hs.77199; ATP synthase, H+ transporting, mitochondrial F0 complex, | |
| | subunit b, isoform 1” | |
| ATP5G1 | “ATP synthase, H+ transporting, mitochondrial F0 complex, subunit c | |
| | (subunit 9), isoform 1; ATP5G” | |
| ATP5G2 | “ATP synthase, H+ transporting, mitochondrial F0 complex, subunit c | |
| | (subunit 9), isoform 2” | |
| ATP5G3 | “ATP synthase, H+ transporting, mitochondrial F0 complex, subunit c | |
| | (subunit 9) isoform 3” | |
| ATP5GP1 | “ATP synthase, H+ transporting, mitochondrial F0 complex, subunit c | |
| | (subunit 9) pseudogene 1” | |
| ATP5H | “ATP synthase, H+ transporting, mitochondrial F0 complex, subunit d” | |
| ATP5I | “ATP synthase, H+ transporting, mitochondrial F0 complex, subunit e (? | |
| | oligomycin sensitivity conferring protein)” | |
| ATP5J | “ATP synthase, H+ transporting, mitochondrial F0 complex, subunit F6” | |
| ATP5J2 | “ATP5JL; F1FO-ATPASE; ATP5J2-PENDING; ATP synthase, H+ | |
| | transporting, mitochondrial F0 complex, subunit f, isoform 2” | |
| ATP5JD | “ATP synthase, H+ transporting, mitochondrial F1F0, subunit d” | |
| ATP5JG | “ATP synthase, H+ transporting, mitochondrial F1F0, subunit g” | |
| ATP5O | “ATP synthase, H+ transporting, mitochondrial F1 complex, O subunit | |
| | (oligomycin sensitivity conferring protein); Hs.76572; OSCP; ATPO” | |
| ATP6A1 | “Hs.52210; VPP2; ATPase, H+ transporting, lysosomal (vacuolar proton | |
| | pump), alpha polypeptide, 70 kD, isoform 1” | |
| ATP6A2 | “Hs.603; VPP2; ATPase, H+ transporting, lysosomal (vacuolar proton | |
| | pump), alpha polypeptide, 70 kD, isoform 2” | |
| ATP6B1 | “ATPase, H+ transporting, lysosomal (vacuolar proton pump), beta | |
| | polypeptide, 56/58 kD, isoforrn 1; Hs.1009; VPP3; V-ATIPASE; VATB” | |
| ATP6B2 | “ATPase, H+ transporting, lysosomal (vacuolar proton pump), beta | |
| | polypeptide, 56/58 kD, isoform 2; Hs.56298; VPP3; Hs.1697” | |
| ATP6C | “Hs.76159; ATPL; ATPase, H+ transporting, lysosomal (vacuolar proton | |
| | pump) 16 kD” | |
| ATP6D | “Hs.86905; ATPase, H+ transporting, lysosomal (vacuolar proton pump) | |
| | 42 kD” | |
| ATP6DV | “vacuolar proton-ATPase, subunit D; V-ATPase, subunit D” | |
| ATP6E | “Hs.77805; ATPase, H+ transporting, lysosomal (vacuolar proton pump) | |
| | 31 kD; Hs.74105" | |
| ATP6EL1 | “ATPase, H+ transporting, lysosomal (vacuolar proton pump) 31 kD-like | |
| | 1” | |
| ATP6EP1 | “ATPase, H+ transporting, lysosomal (vacuolar proton pump) 31 kD | |
| | pseudogene 1” | |
| ATP6EP2 | “ATPase, H+ transporting, lysosomal (vacuolar proton pump) 31 kD | |
| | pseudogene 2” | |
| ATP6F | “ATPase, H+ transporting, lysosomal (vacuolar proton pump) 21 kD” | |
| ATP6G | “ATPase, H+ transporting, lysosomal (vacuolar proton pump)” | |
| ATP6H | “ATPase, H+ transporting, lysosomal (vacuolar proton pump) 9 kD” | |
| ATP6J | “ATPase, H+ transporting, lysosomal (vacuolar proton pump), member | |
| | J; ATP6GL” | |
| ATP6N1A | “ATP6N1; ATPase, H+ transporting, lysosomal (vacuolar proton pump) | |
| | non-catalytic accessory protein 1A (110/116 kD); VPP1; vacuolar proton | |
| | pump, subunit 1” | |
| ATP6N2 | “ATPase, H+ transporting, lysosomal (vacuolar proton pump) non- | |
| | catalytic accessory protein 2 (38 kD)” | |
| ATP6S1 | “ATPase, H+ transporting, lysosomal (vacuolar proton pump), subunit 1; | |
| | ORF; XAP-3; VATPS1; 16A” | |
| ATP6S14 | “ATPase, vacuolar, 14 kD” | |
| ATP7A | “Hs.606; MNK; ATPase, Cu++ transporting, alpha polypeptide (Menkes | |
| | syndrome)” | |
| ATP7B | “ATPase, Cu++ transporting, beta polypeptide (Wilson disease); | |
| | Hs.84999; WND” | |
| ATPC2B | “ATPASEP; ATPase, class 2, member b; ATPase type IV, phospholipid | |
| | transporting (P-type) (putative)” | |
| ATPP2 | ATPASEII; aminophospholipid translocase | |
| ATRN | attractin (with dipeptidylpeptidase IV activity) | |
| AUH | AU RNA-binding protein/enoyl-Coenzyme A hydratase | |
| AXL | Hs.83341; AXL receptor tyrosine kinase | |
| B3GALT1 | “UDP-Gal:betaGlcNAc beta 1,3-galactosyltransferase, polypeptide 1; | |
| | BETA3GAL-T1” | |
| B3GALT2 | “beta-1,3-glucuronyltransferase 2 (glucuronosyltransferase S); | |
| | BETA3GAL-T2; UDP-Gal:betaGlcNAc beta 1,3-galactosyltransferase, | |
| | polypeptide 2; GlcAT-S” | |
| B3GALT3 | “BETA3GAL-T3; UDP-Gal:betaGlcNAc beta 1,3-galactosyltransferase, | |
| | polypeptide 3” | |
| B3GALT4 | “BETA3GAL-T4; UDP-Gal:betaGlcNAc beta 1,3-galactosyltransferase, | |
| | polypeptide 4” | |
| B3GALT5 | “UDP-Gal:betaGlcNAc beta 1,3-galactosyltransferase, polypeptide 5; | |
| | beta3Gal-T5” | |
| B4GALT1 | GGTB2; Hs.80881; glycoprotein-4-beta-galactosyltransferase 2 | |
| B4GALT2 | “UDP-Gal:betaGlcNAc beta 1,4-galactosyltransferase, polypeptide 2; | |
| | beta4Gal-T2” | |
| B4GALT3 | “BETA4GAL-T3; UDP-Gal:betaGlcNAc beta 1,4-galactosyltransferase, | |
| | polypeptide 3” | |
| B4GALT4 | “BETA4GAL-T4; UDP-Gal:betaGlcNAc beta 1,4-galactosyltransferase, | |
| | polypeptide 4” | |
| B4GALT5 | “UDP-Gal:betaGlcNAc beta 1,4-galactosyltransferase, polypeptide 5; | |
| | beta4GalT-V; beta4-GalT IV” | |
| B4GALT6 | “UDP-Gal:betaGlcNAc beta 1,4-galactosyltransferase, polypeptide 6” | |
| B4GALT7 | “xylosylprotein betal,4-galactosyltransferase, polypeptide 7 | |
| | (galactosyltransferase I); XGPT1; XGALT-1; beta4Gal-T7” | |
| B99 | GTSE-1; Hs.122552; Gtsel (mouse) homolog; GTSE1; G two S phase | |
| | expressed protein 1 | |
| BAAT | BAT; bile acid Coenzyme A: amino acid N-acyltransferase (glycine N- | |
| | choloyltransferase) | |
| BAP1 | BRCA1 associated protein-1 (ubiquitin carboxy-terminal hydrolase); | |
| | ubiquitin carboxy-terminal hydrolase | |
| BBOX | “BBH; G-BBH; GAMMA-BBH; butyrobetaine (gamma), 2-oxoglutarate | |
| | dioxygenase (gamma-butyrobetaine hydroxylase)” | |
| BCAT1 | “BCT1; branched chain aminotransferase 1, cytosolic” | |
| BCAT2 | “BCT2; branched chain aminotransferase 2, mitochondrial” | |
| BCHE | butyrylcholinesterase; E1; CHE1 | |
| BCHEL1 | butyrylcholinesterase-like 1; CHEL1 | |
| BCHEL3 | butyrylcholinesterase-like 3; CHEL3 | |
| BCKDHA | “Hs.78950; branched chain keto acid dehydrogenase E1, alpha | |
| | polypeptide (maple syrup urine disease)” | |
| BCKDHB | “Hs.1265; branched chain keto acid dehydrogenase E1, beta polypep- | |
| | tide (maple syrup urine disease)” | |
| BCKDK | branched chain aipha-ketoacid dehydrogenase kinase | |
| BCPM | benign chronic pemphigus (Hailey-Hailey disease) | |
| BDH | “3-hydroxybutyrate dehydrogenase (heart, mitochondrial)” | |
| BETA3GNT | “beta-1,3-N-acetylglucosaminyltransferase” | |
| BETA3GNTI | “i-beta-1,3-N-acetylglucosaminyltransferase” | |
| BHMT | betaine-homocysteine methyltransferase | |
| BLK | B lymphoid tyrosine kinase; Hs.2243 | |
| BLMH | bleomycin hydrolase | |
| BLVRA | BLVR; biliverdin reductase A | |
| BLVRB | biliverdin reductase B | |
| BMPR1A | “ACVRLK3; bone morphogenetic protein receptor, type IA; ALK3; | |
| | activin A receptor, type II-like kinase 3” | |
| BMPR2 | “bone morphogenetic protein receptor, type II (serine/threonine kinase); | |
| | BRK-3; T-ALK; BMPR3; BMPR-II” | |
| BMX | BMX non-receptor tyrosine kinase; ETK; PSCTK2 | |
| BPGM | “Hs.79537; 2,3-bisphosphoglycerate mutase” | |
| BPHL | biphenyl hydrolase-like (serine hydrolase); D0S2254E; MCNAA; Bph-rp | |
| BPNT1 | “3′(2′), 5′-bisphosphate nucleotidase 1” | |
| BTD | Hs.78885; biotinidase | |
| BTK | Bruton agammaglobulinemia tyrosine kinase; ATK; XLA; IMD1; | |
| | AGMX1; PSCTK1 | |
| CA1 | Hs.23118; carbonic anhydrase I | |
| CA10 | carbonic anhydrase X | |
| CA11 | carbonic anhydrase XI; CARP2 | |
| CA12 | carbonic anhydrase XII | |
| CA2 | Hs.89748; carbonic anhydrase II; Hs.78883 | |
| CA3 | “carbonic anhydrase III, muscle specific” | |
| CA4 | carbonic anhydrase IV; Hs.89485; CAIV | |
| CA5A | “CA5; carbonic anhydrase VA, mitochondrial; carbonic anhydrase V, | |
| | mitochondrial; Hs.137; CAV; CAVA” | |
| CA5B | “carbonic anhydrase VB, mitochondrial” | |
| CA5P | carbonic anhydrase V pseudogene | |
| CA6 | Hs.73855; carbonic anhydrase VI | |
| CA7 | carbonic anhydrase VII | |
| CA8 | carbonic anhydrase VIII; CALS; CARP | |
| CA9 | carbonic anhydrase IX; MN | |
| CAD | “carbamoyl-phosphate synthetase 2, aspartate transcarbamylase, and | |
| | dihydroorotase” | |
| CALM1 | “calmodulin 1 (phosphorylase kinase, delta); Hs.73785; CAMI; PHKD; | |
| | DD132; CALML2” | |
| CALM1P1 | “calmodulin 1 (phosphorylase kinase, delta) pseudogene 1” | |
| CALM1P2 | “calmodulin 1 (phosphorylase kinase, delta) pseudogene 2” | |
| CALM2 | “PHKD; CAMII; calmodulin 2 (phosphorylase kinase, delta)” | |
| CALM3 | “PHKD; calmodulin 3 (phosphorylase kinase, delta)” | |
| CAMK1 | calcium/calmodulin-dependent protein kinase I; CAMK1-PEN; CaMKI | |
| CAMK2A | CAMKA; calciumlca/modulin-dependent protein kinase (CaM kinase) II | |
| | alpha; KIAA0968 | |
| CAMK2B | CAMKB; calciumlca/modulin-dependent protein kinase (CaM kinase) II | |
| | beta | |
| CAMK2D | CAMKD; calciumlca/modulin-dependent protein kinase (CaM kinase) II | |
| | delta; CaMKII delta | |
| CAMK2G | CAMKG; calcium/calmodulin-dependent protein kinase (CaM kinase) II | |
| | gamma | |
| CAMK4 | calcium/calmodulin-dependent protein kinase IV; Hs.348 | |
| CAMKK1 | “calcium/camodulin-dependent protein kinase kinase 1, alpha; CaMKKa” | |
| CAMK1K2 | “calcium/calmodulin-dependent protein kinase kinase 2, beta; CaMKK; | |
| | CaMKKb; KIAA0787” | |
| CANPX | calpain-like protease | |
| CAP1 | “CAP1-PEN; adenylyl cyclase-associated CAP protein, yeast homolog” | |
| CAP2 | adenylyl cyclase-associated protein 2 | |
| CAPN7 | calpain 7; calpain like protease; PalBH | |
| CARKL | carbohydrate kinase-like | |
| CARM1 | coactivator-associated arginine methyltransferase-1 | |
| CARS | Hs.16642; cysteinyl-tRNA synthetase | |
| CASK | calcium/calmodulin-dependent serine protein kinase (MAGUK family) | |
| CASKP | calcium/calmodulin-dependent serine protein kinase (MAGUK family) | |
| | pseudogene | |
| CASP1 | “IL1BC; caspase 1, apoptosis-related cysteine protease (interleukin 1, | |
| | beta, convertase); Hs.2490; ICE” | |
| CASP10 | “caspase 10, apoptosis-related cysteine protease; MCH4” | |
| CASP13 | “caspase 13, apoptosis-related cysteine protease; ERICE” | |
| CASP2 | “NEDD2; caspase 2, apoptosis-related cysteine protease (neural pre- | |
| | cursor cell expressed, developmentally down-regulated 2); ICH1” | |
| CASP3 | “CPP32B; caspase 3, apoptosis-related cysteine protease; Yama; CPP32; | |
| | apopain” | |
| CASP4 | caspase 4, apoptosis-related cysteine protease; TX; ICH-2; ICErel-II” | |
| CASP5 | “caspase 5, apoptosis-related cysteine protease; ICErel-III” | |
| CASP6 | “caspase 6, apoptosis-related cysteine protease; MCH2” | |
| CASP7 | “caspase 7, apoptosis-related cysteine protease; MCH3; CMH-1; ICE- | |
| | LAP3” | |
| CASP8 | “caspase 8, apoptosis-related cysteine protease; MACH; MCH5; FLICE” | |
| CASP9 | “caspase 9, apoptosis-related cysteine protease; APAF3; MCH6; ICE- | |
| | LAP6” | |
| CAT | Hs.76359; catalase | |
| CAXIV | CA13; carbonic anhydrase 13 | |
| CBR1 | CBR; carbonyl reductase 1; Hs.88778; carbonyl reductase (NADPH) | |
| CBR3 | carbonyl reductase 3 | |
| CBS | cystathionine-beta-synthase; Hs.84152 | |
| CCAL1 | “CPDD; chondrocalcinosis 1 (calcium pyrophosphate-deposition disease, | |
| | early onset osteoarthritis)” | |
| CCAL2 | “chondrocalcinosis 2 (calcium pyrophosphate-deposition disease, without | |
| | osteoarthritis)” | |
| CCBL1 | “cysteine conjugate-beta lyase; cytoplasmic (glutamine transaminase K, | |
| | kyneurenine aminotransferase)” | |
| CCO | central core disease of muscle | |
| CCS | copper chaperone for superoxide dismutase | |
| CDA | Hs.72924; CDD; cytidine deaminase | |
| CDC20 | “cell division cycle 20, S. cerevisiae homolog; p55CDC; protein kinase | |
| | associated protein, similar to s. cerevisiae cell division cycle proteins | |
| | Cdc20 and Cdc4; P55CDC-LSB” | |
| CDC2L5 | cell division cycle 2-like 5 (cholinesterase-related cell division | |
| | controller); CDC2L; CHED | |
| CDC42BPA | MRCK; MRCKA; CDC42-binding protein kinase alpha (DMPK-like) | |
| CDC42BPB | MRCKB; CDC42-binding protein kinase beta (DMPK-like) | |
| CDC42GA1 | CDC42GA1-PEN; CDC42 GTPase activating protein 1 | |
| CDK10 | “PISSLRE; protein kinase, serine/threonine cdc2-related” | |
| CDK2 | Hs.99981; cyclin-dependent kinase 2; Hs.19192 | |
| CDK3 | cyclin-dependent kinase 3 | |
| CDK4 | PSK-J3; cyclin-dependent kinase 4 | |
| CDK5 | Hs.2869; PSSALRE; cyclin-dependent kinase 5 | |
| CDK5R1 | “cyclin-dependent kinase 5, regulatory subunit 1 (p35); CDK5P35; p35; | |
| | Nck5a; p35nck5a” | |
| CDK5R2 | “cyclin-dependent kinase 5, regulatory subunit 2 (p39); cyclin-dependent | |
| | kinase 5, regulatory subunit 2 (p39); p39; p39nck5ai” | |
| CDK6 | cyclin-dependent kinase 6; Hs.38481; PLSTIRE | |
| CDK7 | Hs.83088; CAK1; CDKN7; cyclin-dependent kinase 7 (homolog of | |
| | Xenopus MO15 cdk-activating kinase); STK1 | |
| CDK8 | cyclin-dependent kinase 8; K35 | |
| CDK9 | CDC2L4; cyclin-dependent kinase 9 (CDC2-related kinase); PITALRE; | |
| | TAR; C-2k | |
| CDKL1 | 1 cyclin-dependent kinase-like 1 (CDC2-related kinase); KKIALRE | |
| CDKL2 | cyclin-dependent kinase-like 2 (CDC2-related kinase); P56; KKIAMRE; | |
| | cyclin-dependent kinase-like 2 (CDC2-related kinase) | |
| CDKN1A | “cyclin-dependent kinase inhibitor 1A (p21, Cip1); Hs.74984; P21; CIP1; | |
| | WAF1; SDI1; CDKN1; CAP20” | |
| CDKN1B | “KIP1; P27KIP1; cyclin-dependent kinase inhibitor 1B (p27, Kip1)” | |
| CDKN1C | “P57; KIP2; cyclin-dependent kinase inhibitor 1C (p57, Kip2)” | |
| CDKN2A | “CDKN2; cyclin-dependent kinase inhibitor 2A (melanoma, p16, inhibits | |
| | CDK4); CDK4I; MLM; Hs.1174; P16; INK4; MTS1; CMM2” | |
| CDKN2B | “P15; MTS2; INK4B; cyclin-dependent kinase inhibitor 2B (p15, inhib- | |
| | its CDK4)” | |
| CDKN2C | “INK4C; cyclin-dependent kinase inhibitor 2C (p18, inhibits CDK4)” | |
| CDKN2D | “cyclin-dependent kinase inhibitor 2D (p19, inhibits CDK4); INK4D” | |
| CDKN3 | cyclin-dependent kinase inhibitor 3 (CDK2-associated dual specificity | |
| | phosphatase); KAP; CDI1 | |
| CDO1 | cysteine dioxygenase type I; Hs.3229 | |
| CDS1 | CDP-diacylglycerol synthase (phosphatidate cytidylyltransferase) 1 | |
| CDS2 | CDP-diacylglycerol synthase (phosphatidate cytidylyltransferase) 2 | |
| CEL | Hs.99918; BSSL; carboxyl ester lipase (bile salt-stimulated lipase) | |
| CELL | Hs.257; carboxyl ester lipase-like (bile salt-stimulated lipase-like) | |
| CEPT1 | choline/ethanolaminephosphotransferase | |
| CES1 | CES2; carboxylesterase 1 (monocyte/macrophage senne esterase 1); | |
| | carboxylesterase 2 (liver); SES1; Hs.76688; HMSE; HMSE1 | |
| CES2 | “carboxylesterase 2 (intestine, liver); intestinal carboxylesterase; liver | |
| | carboxylesterase-2; iCE; CE-2; hCE-2” | |
| CH25H | cholesterol 25-hydroxylase; C25H | |
| CHAT | choline acetyltransferase | |
| CHD1 | chromodomain helicase DNA binding protein 1 | |
| CHD1L | CHDL; CHD1L-PENDING; chromodomain helicase DNA binding | |
| | protein 1-like | |
| CHD2 | chromodomain helicase DNA binding protein 2 | |
| CHD3 | chromodomain helicase DNA binding protein 3; Mi-2a | |
| CHD4 | chromodomain helicase DNA binding protein 4; Mi-2b | |
| CHDRL1 | CHDRL1-PEN; chlordecone reductase-like 1 | |
| CHDRL2 | CHDRL2-PEN; chiordecone reductase-like 2 | |
| CHDRL3 | CHDRL3-PEN; chiordecone reductase-like 3 | |
| CHE2 | cholinesterase (serum) 2 | |
| CHI3L1 | chitinase 3-like 1; HCGP-3P; GP39; YKL40; YKL-40 | |
| CHI3L2 | chitinase 3-like 2 | |
| CHIT1 | “chitinase 1; chitinase, chitotriosidase; CHIT-LSB; Hs.79115; CHIT” | |
| CHK | Hs.77221; CKI; choline kinase | |
| CHKL | choline kinase-like | |
| CHST1 | carbohydrate (chondroitin 6/keratan) sulfotransferase 1; C6ST; KSGal6ST | |
| CHST2 | carbohydrate (chondroitin 6/keratan) sulfotransferase 2 | |
| CHST3 | carbohydrate (chondroitin 6/keratan) sulfotransferase 3; C6ST; | |
| | carbohydrate (chondroitin 6/keratan) sulfotransferase 3 | |
| CHST4 | carbohydrate (N-acetylglucosamine 6-O) sulfotransferase 4; HEC- | |
| | GLCNAC-6-ST; N-acetylglucosamine 6-O-sulfotransferase; LSST | |
| CHUK | conserved helix-loop-helix ubiquitous kinase; IKK1; NFKBIKA; | |
| | IkBKA; IKK-alpha; TCF16 | |
| CILP | “cartilage intermediate layer protein, nucleotide pyrophosphohydrolase” | |
| CIT | “CRIK; STK21; KIAA0949; citron (rho-interacting, serine/theorine | |
| | kinase 21)” | |
| CKB | “Hs.669; CKBB; creatine kinase, brain” | |
| CKBE | “creatine kinase, ectopic expression” | |
| CKBP1 | creatine kinase B pseudogene 1 | |
| CKM | “creatine kinase, muscle; Hs.75635; CKMM” | |
| CKMT1 | “CKMT; UMTCK; creatine kinase, mitochondrial 1 (ubiquitous)” | |
| CKMT2 | “Hs.80691; SMTCK; creatine kinase, mitochondrial 2 (sarcomeric)” | |
| CKS1 | CDC28 protein kinase 1; Hs.77550; CRS1 (S. cerevisiae; Cdc28/Cdc2 | |
| | kinase subunit) homolog-1 | |
| CRS2 | CDC28 protein kinase 2; Hs.83758; CKS1 (S. cerevisiae Cdc28/Cdc2 | |
| | kinase subunit) homolog-2 | |
| CLCN1 | “CLC1; chloride channel 1, skeletal muscle (Thomsen disease, auto- | |
| | somal dominant)” | |
| CLCN5 | “NPHL2; chloride channel 5; Hs.3121; DENTS; nephrolithiasis 2 (X- | |
| | linked, Dent disease)” | |
| CLK1 | CLK; CDC-like kinase | |
| CLK2 | CDC-like kinase 2 | |
| CLK2P | “CDC-like kinase 2, pseudogene” | |
| CLK3 | CDC-like kinase 3 | |
| CLN2 | “ceroid-lipofuscinosis, neuronal 2, late infantile (Jansky-Bielschowsky | |
| | disease)” | |
| CLN3 | “ceroid-lipofuscinosis, neuronal 3, juvenile (Batten, Spielmeyer-Vogt | |
| | disease); Hs.77479; BTS” | |
| CLN4 | “ceroid-lipofuscinosis, neuronal 4 (Kufs disease)” | |
| CLPP | “ClpP (caseinolytic protease, ATP-dependent, proteolytic subunit, E. coli) | |
| | homolog” | |
| CLPS | “Hs.1340; colipase, pancreatic” | |
| CLPX | “ClpX (caseinolytic protease X, E. coli) homolog; energy-dependent | |
| | regulator of proteolysis” | |
| CMA1 | “chymase 1, mast cell” | |
| CMAH | cytidine monophosphate-N-acetylneuraminic acid hydroxylase (CMP-N- | |
| | acetylneuraminate monooxygenase) | |
| CMAS | CYTIDINE 5-PRIME-MONOPHOSPHATE N-ACETYLNEURAMINIC | |
| | ACID SYNTHETASE | |
| CNK | cytokine-inducible kinase; FNK; PRK | |
| CNK1 | KSR; connector enhancer of KSR-like (Drosophila kinase suppressor of | |
| | ras) | |
| CNP | “Hs.75062; 2′,3′-cyclic nucleotide 3′phosphodiesterase” | |
| CNSN | Camosinemia (carnosinase) | |
| COLQ | collagen-like tail subunit (single strand of homotrimer) of asymmetnc | |
| | acetylcholinesterase | |
| COMT | Hs.89893; catechol-O-methyltransferase; Hs.78534 | |
| COX10 | cytochrome c oxidase subunit X (heme A: farnesyltransferase); Hs.77513 | |
| COX11 | cytochrome c oxidase subunit 11 | |
| COX11P | “cytochrome c oxidase subunit 11, pseudogene” | |
| COX15 | cytochrome c oxidase subunit 15 | |
| COX17 | “COX17 (yeast) homolog, cytochrome c oxidase assembly protein; human | |
| | homolog of yeast mitochondrial copper recruitment gene” | |
| COX17P | “COX17 (yeast) homolog, cytochrome c oxidase assembly protein, | |
| | pseudogene” | |
| COX4 | Hs.686; cytochrome c oxidase subunit IV | |
| COX4P1 | COX4L1; cytochrome c oxidase subunit IV pseudogene 1 | |
| COX5A | VA; COX; COX-VA; cytochrome c oxidase subunit Va | |
| COX5AP1 | cytochrome c oxidase subunit Va pseudogene I | |
| COX5B | Hs.1342; cytochrome c oxidase subunit Vb | |
| COX5BL1 | cytochrome c oxidase subunit Vb-like 1 | |
| COX5BL2 | cytochrome c oxidase subunit Vb-like 2 | |
| COX5BL3 | cytochrome c oxidase subunit Vb-like 3 | |
| COX5BL4 | cytochrome c oxidase subunit Vb-like 4 | |
| COX5BL5 | cytochrome c oxidase subunit Vb-like 5 | |
| COX5BL6 | cytochrome c oxidase subunit Vb-like 6 | |
| COX5BL7 | cytochrome c oxidase subunit Vb-like 7 | |
| COX6A1 | COX6A; cytochrome c oxidase subunit VIa polypeptide 1 | |
| COX6A1P | cytochrome c oxidase subunit VIa polypeptide 1 pseudogene | |
| COX6A2 | cytochrome c oxidase subunit VIa polypeptide 2 | |
| COX6B | Hs.83379; cytochrome c oxidase subunit VIb | |
| COX6BP1 | cytochrome c oxidase subunit VIb pseudogene 1 | |
| COX6BP2 | cytochrome c oxidase subunit VIb pseudogene 2 | |
| COX6BP3 | cytochrome c oxidase subunit VIb pseudogene 3 | |
| COX6BP4 | cytochrome c oxidase subunit VIb pseudogene 4 | |
| COX6C | Hs.74649; cytochrome c oxidase subunit VIc | |
| COX6CP1 | cytochrome c oxidase subunit VIc pseudogene 1 | |
| COX7A1 | cytochrome c oxidase subunit VIIa polypeptide 1 (muscle); Hs.71883; | |
| | COX7A | |
| COX7A2 | Hs.2321; cytochrome c oxidase subunit VIIa polypeptide 2 (liver) | |
| COX7A3 | cytochrome c oxidase subunit VIIa polypeptide 3 (liver) | |
| COX7B | Hs.75752; cytochrome c oxidase subunit VIIb | |
| COX7C | Hs.3462; cytochrome c oxidase subunit VIIc | |
| COX7CP1 | cytochrome c oxidase subunit VIIc pseudogene 1 | |
| COX7RP | cytochrome c oxidase subunit VII-related protein | |
| COX8 | cytochrome c oxidase subunit VIII | |
| CP | Hs.10735; ceruloplasmin (ferroxidase) | |
| CPA1 | Hs.2879; CPA; carboxypeptidase Al (pancreatic) | |
| CPA2 | carboxypeptidase A2 (pancreatic) | |
| CPA3 | Hs.646; carboxypeptidase A3 (mast cell) | |
| CPB1 | carboxypeptidase BT (tissue); Hs.56117 | |
| CPB2 | carboxypeptidase B2 (plasma); CPU; carboxypeptidase U; Hs.75572; | |
| | PCPB | |
| CPD | carboxypeptidase D | |
| CPE | Hs.75360; carboxypeptidase E | |
| CPM | Hs.50997; carboxypeptidase M | |
| CPN1 | “carboxypeptidase N, polypeptide 1, 50 kD; CPNE1” | |
| CPN2 | “ACBP; carboxypeptidase N, polypeptide 2, 83 kD; Hs.2246; arginine | |
| | carbox eptidase (carboxypeptidase N)” | |
| CPO | “Hs.89866; CPX; coproporphyrinogen oxidase (coproporphyria, | |
| | harderoporphyria); Hs.79904” | |
| CPP | ceruloplasmin (ferroxidase) pseudogene | |
| CPS1 | “Hs.50966; carbamoyl-phosphate synthetase 1, mitochondrial” | |
| CPT1A | “CPT1; carnitine palmitoyltransferase I, liver; CPT1-L; L-CPT1” | |
| CPT1B | “carnitine palmitoyltransferase I, muscle; M-CPT1; CPT1-M” | |
| CPT2 | 1; CPTT; CPTASE; camitine palmitoyltransferase II | |
| CPZ | carboxypeptidase Z | |
| CRAT | Hs.12068; CAT1; carnitine acetyltransferase | |
| CRMP1 | collapsin response mediator protein 1 (dihydropyrimidinase-like 1); | |
| | DRP- 1; DPYSL1; Hs.75079 | |
| CRY1 | PHLL1; cryptoebrome 1 (photolyase-like) | |
| CRY2 | cryptochrome 2 (photolyase-like) | |
| CRYZ | “Hs.83114; crystallin, zeta (quinone reductase)” | |
| CRYZL1 | “crystallin, zeta (guinone reductase)-like 1” | |
| CRYZP1 | “crystallin, zeta (guinone reductase) pseudogene 1” | |
| CS | citrate synthase | |
| CSCI | Corticosterone side-chain isomerase | |
| CSK | Hs.89756; c-src tyrosine kinase; Hs.77793 | |
| CSN1 | “casein, alpha; Hs.3155; CASA” | |
| CSN10 | “casein, kappa; CSN3” | |
| CSN2 | “casein, beta; Hs.2242; CASB” | |
| CSNK1A1 | “Hs.52195; casein kinase 1, alpha 1” | |
| CSNK1D | “casein kinase 1, delta; Hs.75852; HCKID” | |
| CSNK1E | “casein kinase 1, epsilon; Hs.79658; CKIe; HCKIE” | |
| CSNK1G2 | “casein kinase 1, gamma 2” | |
| CSNK1G3 | “casein kinase 1, gamma 3” | |
| CSNK2A1 | “Hs.12740; casein kinase 2, alpha 1 polypeptide” | |
| CSNK2A1P | “casein kinase 2, alpha 1 polypeptide pseudogene” | |
| CSNK2A2 | “Hs.82201; CSNK2A1; casein kinase 2, alpha prime polypeptide” | |
| CSNK2B | “Hs.84316; casein kinase 2, beta polypeptide” | |
| CST | cerebroside (3′-phosphoadenylylsulfate:galactosylceramide 3′) | |
| | sulfotransferase | |
| CTBS | “CTB; chitobiase, di-N-acetyl-; Hs.99889” | |
| CTD | Coats disease | |
| CTDP1 | “CTD (carboxy-terminal domain, RNA polymerase II, polypeptide A) | |
| | phosphatase, subunit 1; FCP1; CTD (carboxy-terminal domain, RNA | |
| | polymerase II, polypeptide A) phosphatase, subunit 1” | |
| CTH | Hs.19904; cystathionase (cystathionine gamma-lyase) | |
| CTPS | Hs.84112; CTP synthase | |
| CTRC | “chymotrypsin C (caldecrin); caldecrin (serum calcium decreasing factor, | |
| | elastase IV); CLCR” | |
| CTSD | Hs.79572; CPSD; cathepsin D (lysosomal aspartyl protease) | |
| CYBB | “cytochrome b-245, beta polypeptide (chronic granulomatous disease); | |
| | Hs.88974; CGD; GP91-PHOX” | |
| CYP11B1 | “cytochrome P450, subfamily XIB (steroid 11-beta-hydroxylase), | |
| | polypeptide 1; Hs.2610; CYP11B” | |
| CYP11B2 | “cytochrome P450, subfamily XIB (steroid 11-beta-hydroxylase), | |
| | polypeptide 2; Hs.36986; CYP11B” | |
| CYP17 | “Hs.1363; cytochrome P450, subfamily XVII (steroid 17-alpha- | |
| | hydroxylase), adrenal hyperplasia” | |
| CY1P19 | “cytochrome P450, subfamily XIX (aromatization of androgens); | |
| | Hs.79946; aromatase“ | |
| CYP21A1P | “CYP21P; cytochrome P450, subfamily XXIA (steroid 21-hydroxylase), | |
| | polypeptide 1 pseudogene; CYP21A; cytocbrome P450, subfamily XXI | |
| | (steroid 21-hydroxylase) pseudogene; P450c21A” | |
| CYP21A2 | “CYP21; cytochrome P450, subfamily XXIA (steroid 21-hydroxylase, | |
| | congenital adrenal hyperplasia), polypeptide 2; Hs.49066; CYP21B; | |
| | cytochrome P450, subfamily XXI (steroid 21-hydroxylase, congenital | |
| | adrenal hyperplasia); P450c21B” | |
| CYP24 | “cytochrome P450, subfamily XXIV (vitamin D 24-hydroxylase)” | |
| CYP27A1 | “CYP27; cytochrome P450, subfamily XXVIIA (steroid 27-hydroxylase, | |
| | cerebrotendinous xanthomatosis), polypeptide 1; Hs.82568; cytochrome | |
| | P450, subfamily XXVII (sterol 27-hydroxylase, cerebrotendinous | |
| | xanthomatosis)” | |
| CYP27B1 | “PDDR; cytochrome P450, subfamily XXVIIB (25-hydroxyvitamin D-l- | |
| | alpha-hydroxylase), polypeptide 1; VDR; VDD1; pseudo-vitamin D | |
| | dependency rickets 1; CYP1; P450c1; VDDR I” | |
| CYP2C | “Hs.703; cytoclirome P450, subfamily IIC (mephenytoin 4-hydroxylase” | |
| CYP2C10 | “cytochrome P450, subfamily IIC (mephenytoin 4-hydroxylase), | |
| | polypeptide 10” | |
| CYP2C18 | “CYP2C17; cytochrome P450, subfamily IIC (mephenytoin 4- | |
| | hydroxylase), polypeptide 18; Hs.702; P450IIC17; cytoebrome P450, | |
| | subfamily IIC (mephenytoin 4-hydroxylase), polypeptide 17” | |
| CYP2C19 | “cytochrome P450, subfamily IIC (mephenytoin 4-hydroxylase), | |
| | polypeptide 19; P450IIC19” | |
| CYP2C8 | “cytochrome P450, subfamily IIC (mephenytoin 4-hydroxylase), | |
| | polypeptide 8” | |
| CYP2C9 | “cytochrome P450, subfamily IIC (mephenytoin 4-hydroxylase), | |
| | polypeptide 9; Hs.9669; P450IIC9” | |
| CYP2J2 | “cytochrome P450, subfamily IIJ (arachidonic acid epoxygenase), | |
| | polypeptide 2; Hs.30894” | |
| CYP3A | “CYP3; cytochrome P450, subfamily IIIA (niphedipine oxidase)” | |
| CYP3A3 | “Hs.73725; cytochrome P450, subfamily IIIA (niphedipine oxidase), | |
| | polypeptide 3” | |
| CYP3A4 | “cytochrome P450, subfamily IIIA (niphedipine oxidase), polypeptide 4; | |
| | Hs.45” | |
| CYP3A5 | “cytochrome P450, subfamily IIIA (niphedipine oxidase), polypeptide 5; | |
| | Hs.146” | |
| CYP3A5P1 | “cytochrome P450, subfamily IIIA (niphedipine oxidase), pseudogene 1” | |
| CYP46 | “cytochrome P450, subfamily 46 (cholesterol 24-hydroxylase)” | |
| CYP4F3 | “LTB4H; cytochrome P450, subfamily IVF, polypeptide 3 (leukotriene | |
| | B4 omega hydroxylase); leukotriene B4 omega hydroxylase (cytochrome | |
| | P450, subfamily IVF); Hs.101; CYP4F” | |
| CYP51 | “cytochrome P450, 51 (lanosterol 14-alpha-demethylase); Hs.2379” | |
| CYP7A1 | “CYP7; cytochrome P450, subfamily VIIA (cholesterol 7 alpha- | |
| | monooxygenase), polypeptide 1; Hs.1644; cholesterol 7-alpha- | |
| | hydroxylase” | |
| CYP7B1 | “cytochrome P450, subfamily VIIB (oxysterol 7 alpha-hydroxylase), | |
| | polypeptide 1” | |
| CYP8B1 | “cytochrome P450, subfamily VIIB (sterol 12-alpha-hydroxylase), | |
| | polypeptide 1; CYP12” | |
| DAO | Hs.2625; DAMOX; D-amino-acid oxidase | |
| DAPK1 | DAPK; death-associated protein kinase 1 | |
| DAPK3 | death-associated protein kinase 3 | |
| DBH | Hs.2301; dopamine beta-hydroxylase (dopamine beta-monooxygenase) | |
| DBT | Hs.89685; dihydrolipoamide branched chain transacylase (E2 component | |
| | of branched chain keto acid dehydrogenase complex; maple syrup urine | |
| | disease); Hs.23443; Hs.89479 | |
| DCI | “Hs.89466; dodecenoyl-Coenzyme A delta isomerase (3,2 trans-enoyl- | |
| | Coenzyme A isomerase)” | |
| DCK | Hs.709; deoxycytidine kinase | |
| DCT | “Hs.23454; TYRP2; dopachrome tautomerase (dopachrome delta- | |
| | isomerase, tyrosine-related protein 2); Hs.472” | |
| DCTD | Hs.76894; dCMP deaminase | |
| DDAH1 | dimethylarginine dimethylaminohydrolase 1; DDAH; DDAHI | |
| DDAH2 | dimethylarginine dimethylaminohydrolase 2; DDAHII | |
| DDC | Hs.475; dopa decarboxylase (aromatic L-amino acid decarboxylase) | |
| DDO | D-aspartate oxidase | |
| DDOST | dolichyl-diphosphooligosaccharide-protein glycosyltransferase; OST | |
| DDR1 | “NEP; CAK; EDDR1; NTRK4; PTK3A; PTK3A protein tyrosine kinase | |
| | 3A; neurotrophic tyrosine kinase, receptor, type 4; Hs.75562; | |
| | neuroepithelial tyrosine kinase; cell adhesion kinase; trkE; RTK6; | |
| | epithelial discoidin domain receptor 1” | |
| DDR2 | “NTRKR3; TKT; TYRO10; neurotrophic tyrosine kinase, receptor- | |
| | related 3” | |
| DDT | D-dopachrome tautomerase | |
| DDX10 | DEAD/H (Asp-Glu-Ala-Asp/His) box polypeptide 10 (RNA helicase); | |
| | HRH-J8 | |
| DDX11 | DEAD/H (Asp-Glu-Ala-Asp/His) box polypeptide 11 (S. cerevisiae CHL1- | |
| | like helicase); CHLR1 | |
| DDX12 | DEAD/H (Asp-Glu-Ala-Asp/His) box polypeptide 12 (S. cerevisiae CHL1- | |
| | like helicase); CHLR2 | |
| DDX5 | “HLR1; G17P1; DEAD/H (Asp-Glu-Ala-Asp/His) box polypeptide 5 | |
| | (RNA helicase, 68 kD)” | |
| DDX6 | “RCK; HLR2; DEAD/H (Asp-Glu-Ala-Asp/His) box polypeptide 6 (RNA | |
| | helicase, 54 kD)” | |
| DDX7 | “DEAD/H (Asp-Glu-Ala-Asp/His) box polypeptide 7 (RNA helicase, | |
| | 52 kD) NOTE: Symbol and name provisional” | |
| DDX8 | DEAD/H (Asp-Glu-Ala-Asp/His) box polypeptide 8 (RNA helicase); | |
| | HRH1 | |
| DDX9 | “DEAD/H (Asp-Glu-Ala-Asp/His) box polypeptide 9 (RNA helicase A, | |
| | nuclear DNA helicase I ; NDHII” | |
| DDX9P | DEAD/H (Asp-Glu-Ala-Asp/His) box polypeptide 9 (RNA helicase A) | |
| | pseudogene | |
| DDXL | “nuclear RNA helicase, DECD variant of DEAD box family” | |
| DECR | “2,4-dienoyl CoA reductase” | |
| DFFB | “DNA fragmentation factor, 40 kD, beta polypeptide (caspase-activated | |
| | DNase); DNA fragmentation factor, 40 kD, beta subunit; CAD; DFF2; | |
| | CPAN; DFF40; DFF-40” | |
| DGAT | diacylglycerol O-acyltransferase (mouse) homolog; ARGP1 | |
| DGKA | “DAGK1; diacylglycerol kinase, alpha (80 kD); Hs.74044; DGK-alpha; | |
| | DAGK” | |
| DGKB | “DAGK2; diacylglycerol kinase, beta (90 kD); KIAA0718” | |
| DGKD | “diacylglycerol kinase, delta (130 kD); DAGK4-PEN; KIAA0145; | |
| | DGKdelta” | |
| DGKE | “diacylglycerol kinase, epsilon (64 kD); DAGK6; DAGK6-PEN” | |
| DGKG | “DAGK3; diacylglycerol kinase, gamma (90 kD); Hs.89462” | |
| DGKH | “DGKETA; diacylglycerol kinase, eta” | |
| DGKI | “diacylglycerol kinase, iota” | |
| DGKQ | “DAGK4; diacylglycerol kinase, theta (110 kD); diacylglycerol kinase, | |
| | delta (110 kD); Hs.89979; DAGK; DAGK7” | |
| DGKZ | “diacylglycerol kinase, zeta (104 kD); DAGK5; DAGK5-PEN; hDGKzeta” | |
| DGUOK | deoxyguanosine kinase; Hs.101519; dGK; Hs.77494 | |
| DHCR24 | 24-dehydrocholesterol reductase | |
| DHCR7 | 7-dehydrocholesterol reductase | |
| DHFR | Hs.83765; dihydrofolate reductase | |
| DHFRP1 | Hs.73878; dihydrofolate reductase pseudogene 1 | |
| DHFRP2 | dihydrofolate reductase pseudogene 2 | |
| DHFRP4 | dihydrofolate reductase pseudogene 4 | |
| DHODH | Hs.1151; dihydroorotate dehydrogenase | |
| DHPS | deoxyhypusine synthase; Hs.79064 | |
| DIA1 | diaphorase (NADH) (cytochrome b-5 reductase) | |
| DIA2 | Diaphorase-2 | |
| DIA4 | “NMOR1; diaphorase (NADH/NADPH) (cytochrome b-5 reductase); | |
| | NMORI; diaphorase (NADH/NADPH); NAD(P)H menadione | |
| | oxidoreductase 1, dioxin-inducible” | |
| DIFF6 | differentiation 6 (deoxyguanosine triphosphate triphosphohydrolase; | |
| | KIAA0158 | |
| DIO1 | “TXDI1; deiodinase, iodothyronine, type I; 5DI; thyroxine deiodinase type | |
| | I (selenoprotein)” | |
| DIO2 | “deiodinase, iodothyronine type II; thyroxine deiodinase type IL; TXDI2” | |
| DIO3 | “TXDI3; deiodinase, iodothyronine type III; thyroxine deiodinase type III | |
| | (selenoprotein)” | |
| DLAT | Hs.74642; DLTA; PDC-E2; dihydrolipoamide S-acetyltransferase (E2 | |
| | component of pyruvate dehydrogenase complex) | |
| DLD | “Hs.74635; LAD; DLDH; dihydrolipoamide dehydrogenase (E3 | |
| | component of pyruvate dehydrogenase complex, 2-oxo-glutarate complex, | |
| | branched chain keto acid dehydrogenase complex)” | |
| DLST | Hs.401; DLTS; dihydrolipoamide S-succinyltransferase (E2 component of | |
| | 2-oxo-glutarate complex) | |
| DLSTP | dihydrolipoamide S-succinyltransferase pseudogene (E2 component of 2- | |
| | oxo-glutarate complex) | |
| DMPK | DM; DM1; dystrophia myotonica-protein kinase; dystrophia myotonica 1 | |
| | (includes dystrophia myotonia protein kinase); Hs.898 | |
| DNA2L | “DNA2 (DNA replication helicase, yeast, homolog)-like” | |
| DNASE1 | DMA; deoxyribonuclease I | |
| DNASE1L1 | DNL1L; deoxyribonuclease I-like 1 | |
| DNASE1L2 | deoxyribonuclease I-like 2 | |
| DNASE1L3 | deoxyribonuclease I-like 3 | |
| DNASE2 | “DNL2; deoxyribonuclease II, lysosomal; DNL; DNase II, lysosomal" | |
| DNMT1 | DNMT; DNA (cytosine-5-)-methyltransferase 1; Hs.77462; DNA | |
| | methyltransferase | |
| DNMT2 | DNA (cytosine-5-)-methyltransferase 2 | |
| DNMT3A | DNA (cytosine-5-)-methyltransferase 3 alpha | |
| DNMT3B | DNA (cytosine-5-)-methyltransferase 3 beta | |
| DNPEP | aspartyl aminopeptidase; DAP | |
| DNTT | “TDT; deoxynucleotidyltransferase, terminal” | |
| DOK1 | docking protein 1 (downstream of tyrosine kinase 12); p62dok | |
| DPAGT1 | DPAGT; DPAGT2; dolichyl-phosphate alpha-N- | |
| | acetylglucosaminyltransferase 1; dolichyl-phosphate N- | |
| | acetylglucosaminephosphotransferase 2 (GlcNAc-1-P transferase); | |
| | UGAT; dolichyl-phosphate alpha-N-acetylglucosaminyltransferase | |
| DPEP1 | Hs.109; dipetidase 1 (renal) | |
| DPM1 | “dolichyl-phosphate mannosyltransferase polypeptide 1, catalytic subunit” | |
| DPM2 | “dolichyl-phosphate mannosyltransferase polypeptide 2, regulatory | |
| | subunit” | |
| DPP3 | dipeptidylpeptidase III | |
| DPP4 | “Hs.44926; CD26; ADCP2; dipeptidylpeptidase IV (CD26, adenosine | |
| | deaminase complexing protein 2)” | |
| DPP6 | Hs.34074; DPPX; dipeptidylpeptidase VI | |
| DPYD | dihydropyrimidine dehydrogenase | |
| DPYS | dihydropyrimidinase; DHPase | |
| DPYSL2 | dihydropyrimidinase-like 2; DHPRP2; DRP-2; CRMP2 | |
| DPYSL3 | dihydropyrimidinase-like 3; DRP-3 | |
| DPYSL4 | ULIP4; dihydropyrimidinase-like 4 | |
| DTYMK | Hs.79006; deoxythymidylate kinase | |
| DUSP1 | HVH1; CL100; PTPN10; dual specificity phosphatase 1; MKP-1 | |
| DUSP11 | PIR1; dual specificity phosphatase 11 (RNA/RNP complex 1-interacting) | |
| DUSP2 | PAC-1; dual specificity phosphatase 2 | |
| DUSP3 | VHR; dual specificity phosphatase 3 (vaccinia virus phosphatase VHl- | |
| | related) | |
| DUSP4 | HVH2; dual specificity phosphatase 4; MKP-2 | |
| DUSP5 | HVH3; dual specificity phosphatase 5 | |
| DUSP6 | dual specificity phosphatase 6; MKP-3; PYSTl | |
| DUSP7 | dual specificity phosphatase 7; MKP-X; PYST2 | |
| DUSP8 | dual specificity phosphatase 8; HVH-5; HB5 | |
| DUSP8P | dual specificity phosphatase 8 pseudogene | |
| DUSP9 | dual specificity phosphatase 9; MKP4; MKP-4 | |
| DUSPP | dual specificity phosphatase pseudogene; HVH4 | |
| DUT | dUTP pyrophosphatase; Hs.82113 | |
| DYRK1A | DYRK; DYRK1 ; MNBH; dual-specificity tyrosine-(Y)-phosphorylation | |
| | regulated kinase; MNB; minibrain (Drosophila) homolog; Hs.103125 | |
| DYRK1B | dual-specificity tyrosine-(Y)-phosphorylation regulated kinase 1B | |
| DYRK2 | dual-specificity tyrosine-(Y)-phosphorylation regulated kinase 2 | |
| DYRK3 | dual-specificity tyrosine-(Y)-phosphorylation regulated kinase 3 | |
| DYRK4 | dual-specificity tyrosine-(Y)-phosphorylation regulated kinase 4 | |
| EBP | CDPX2; emopamil-binding protein (sterol isomerase); phenylalkylamine | |
| | Ca2+ antagonist (emopamil) binding protein; chondrodysplasia punctata 2 | |
| | (X-linked dominant); CPX; CPXD | |
| ECH1 | “enoyl Coenzyme A hydratase 1, peroxisomal” | |
| ECHS1 | “enoyl Coenzyme A hydratase, short chain, 1, mitochondrial; SCEH” | |
| EFNA1 | “EPLG1; ephrin-A1; LERK1; ECKLG; TNFAIP4; eph-related receptor | |
| | tyrosine kinase ligand 1 (tumor necrosis factor, alpha-induced protein 4); | |
| | B61” | |
| EFNA2 | EPLG6; ephrin-A2; ELF-1; LERK6; eph-related receptor tyrosine kinase | |
| | ligand 6 | |
| EFNA3 | EPLG3; ephrin-A3; LERK3; eph-related receptor tyrosine kinase ligand 3; | |
| | Ehk1-L | |
| EFNA4 | EPLG4; ephrin-A4; LERK4; eph-related receptor tyrosine kinase ligand 4 | |
| EFNA5 | EPLG7; ephrin-A5; Hs.37142; AE1; LERK7; eph-related receptor | |
| | tyrosine kinase ligand 7 | |
| EFNB1 | EPLG2; ephrin-B1; LERK2; eph-related receptor tyrosine kinase ligand 2; | |
| | Elk-L | |
| EFNB2 | EPLG5; ephrin-B2; Hs.30942; LERK5; eph-related receptor tyrosine | |
| | kinase ligand 5; Htk-L | |
| EFNB3 | EPLG8; ephrin-B3; eph-related receptor tyrosine kinase ligand 8; | |
| | Hs.26988; LERK-8 | |
| EHHADH | Hs.1531; enoyl-Coenzyme A hydratase/3-hydroxyacyl Coenzyme A | |
| | dehydrogenase | |
| EIF2AK3 | eukaryotic translation initiation factor 2-alpha kinase 3; PEK; WRS; | |
| | PERK; Wolcott-Rallison syndrome | |
| ELA1 | “Hs.21; elastase 1, pancreatic” | |
| ELA2 | “SERP1; elastase 2, neutrophil; Hs.99863; serine protease” | |
| ELA3 | “elastase 3, pancreatic (protease E)” | |
| ELA3B | elastase 3B | |
| ELANH2 | “EI; PI2; protease inhibitor 2 (anti-elastase), monocyte/neutrophil derived” | |
| ELL2 | “ELL-RELATED RNA POLYMERASE II, ELONGATION FACTOR” | |
| EMK1 | ELKL motif kinase 1; MARK2 | |
| ENDOG | endonuclease G | |
| ENDOGL1 | ENGL; endonuclease G-like 1 | |
| ENDOGL2 | ENGL-B; endonuclease G-like 2 | |
| ENO1 | “ENO1L1; enolase 1, (alpha)-like 1; Hs.675; enolase 1, (alpha); PPH; | |
| | phosphopyruvate hydrates” | |
| ENO1P | “enolase 1, (alpha) pseudogene” | |
| ENO2 | “Hs.75675; enolase 2, (gamma, neuronal)” | |
| ENO3 | “enolase 3, (beta, muscle); Hs.99986; ENO-3; Hs.2645” | |
| ENPEP | glutamyl aminopeptidase (aminopeptidase A); Hs.291; gp160 | |
| EPHA1 | EPHT1; EphA1; EPH; EPHT; eph tyrosine kinase 1 (erythropoietin- | |
| | producing hepatoma amplified sequence); ephrin receptor EphA1 | |
| EPHA2 | ECK; EphA2; ephrin receptor EphA2; epithelial cell receptor protein | |
| | tyrosine kinase | |
| EPHA3 | ETK1; EphA3; ETK; HEK; eph-like tyrosine kinase 1 (human embryo | |
| | kinase 1); ephrin receptor EphA3 | |
| EPHA4 | TYRO1; EphA4; TYRO1 protein tyrosine kinase; Hek8; ephrin receptor | |
| | EphA4 | |
| EPHA8 | “EEK; EphA8; eph-, elk-related tyrosine kinase; Hek3; ephrin receptor | |
| | EphA8” | |
| EPHB1 | EPHT2; EphB1; eph tyrosine kinase 2; Elk; Hek6; ephrin receptor EphB1 | |
| EPHB2 | DRT; ERK; EPHT3; EphB2; eph tyrosine kinase 3; developmentally- | |
| | regulated eph-related tyrosine kinase; Hek5; Tyro5; EPHT3; ephrin | |
| | receptor EphB2; elk-related tyrosine kinase | |
| EPHB3 | ETK2; EphB3; HEK2; eph-like tyrosine kinase 2 (human embryo kinase | |
| | 2); Hek2; Tyro6; ephrin receptor EphB3 | |
| EPHB4 | HTK; EphB4; Hs.464; hepatoma transmembrane kinase; Tyro11; ephrin | |
| | receptor EphB4 | |
| EPHX1 | “epoxide hydrolase 1, microsomal (xenobiotic); Hs.89689; EPHX; | |
| | Hs.89649” | |
| EPHX2 | “Hs.113; epoxide hydrolase 2, cytoplasmic” | |
| EPM2A | “epilepsy, progressive myoclonus type 2, Lafora disease (laforin); EPM2; | |
| | MELF” | |
| EPR1 | effector cell protease receptor 1; EPR-1; effector cell protease receptor 1 | |
| EPRS | QARS; QPRS; glutamyl-prolyl-tRNA synthetase | |
| EPX | eosinophil peroxidase; EPX-PEN; EPO; EPP | |
| ERP70 | “ERP72; protein disulfide isomerase related protein (calcium-binding | |
| | protein, intestinal-related)” | |
| ESA4 | esterase A4 | |
| ESAT | esterase activator | |
| ESB3 | esterase B3 | |
| ESD | Hs.82193; esterase D/formylglutathione hydrolase | |
| ETFDH | ETFQO; electron-transferring-flavoprotein dehydrogenase | |
| EXO1 | HEX1; exonuclease 1 | |
| EYA1 | BOR; eyes absent (Drosophila) homolog 1; branchiootorenal syndrome; | |
| | Melnick-Fraser syndrome | |
| F2R | coagulation factor II (thrombin) receptor; TR; CF2R; PAR1; Hs.85889; | |
| | protease-activated receptor 1 | |
| F9 | “coagulation factor IX (plasma thromboplastic component, Christmas | |
| | disease, hemophilia B); Hs.1330; FIX; Factor 9; Factor IX” | |
| FAAH | fatty acid amide hydrolase | |
| FACL1 | “fatty-acid-Coenzyme A ligase, long-chain 1; Hs.89549; Hs.34” | |
| FACL2 | “FACL1; fatty-acid-Coenzyme A ligase, long-chain 2” | |
| FACL3 | “fatty-acid-Coenzyme A ligase, long-chain 3; ACS3” | |
| FACL4 | “fatty-acid-Coenzyme A ligase, long-chain 4” | |
| FACVL1 | “VLCS; VLACS; fatty-acid-Coenzyme A ligase, very long-chain 1” | |
| FADS1 | LLCDL1; linoleoyl-CoA desaturase (delta-6-desaturase)-like 1 | |
| FADS2 | LLCDL2; linoleoyl-CoA desaturase (delta-6-desaturase)-like 2 | |
| FADS3 | LLCDL3; linoleoyl-CoA desaturase (delta-6-desaturase)-like 3 | |
| FADSD6 | delta-6 fatty acid desaturase | |
| FAH | Hs.73875; fumarylacetoacetase; fumarylacetoacetate | |
| FAK2 | “focal adhesion kinase 2; cell adhesion kinase, beta; PKB; PYK2; | |
| | RAFTK; CAK beta; proline-rich tyrosine kinase 2; CAKB” | |
| FARS1 | phenylalamine-tRNA synthetase | |
| FARSL | phenylalamine-tRINA synthetase-like; CML33 | |
| FASN | fatty acid synthase | |
| FASTK | Fas-activated serine/threonine kinase | |
| FBP1 | Hs.574; FBP; fructose-bisphosphatase 1 | |
| FBP2 | “fructose-1,6-bisphosphatase 2” | |
| FDFT1 | farnesyl-diphosphate famesyltransferase 1; Squalene synthase | |
| FDH | formaldehyde dehydrogenase | |
| FDPS | “Hs.99926; farnesyl diphosphate synthase (farnesyl pyrophosphate | |
| | synthetase, dimethylallyltranstransferase, geranyltranstransferase); | |
| | Hs.123; Hs.99866” | |
| FDPSL1 | “FPSL1; CHR39A; farnesyl diphosphate synthase-like 1 (farnesyl | |
| | pyrophosphate synthetase-like 1, cholesterol-repressible protein 39A)” | |
| FDPSL2 | FPSL2; farnesyl diphosphate synthase-like 2 (farnesyl pyrophosphate | |
| | synthetase-like 2) | |
| FDPSL3 | FPSL3; farnesyl diphosphate synthase-like 3 (farnesyl pyrophosphate | |
| | synthetase-like 3) | |
| FDPSL4 | FPSL4; farnesyl diphosphate synthase-like 4 (farnesyl pyrophosphate | |
| | synthetase-like 4) | |
| FDPSL5 | FPSL5; farnesyl diphosphate synthase-like 5 (farnesyl pyrophosphate | |
| | synthetase-like 5) | |
| FDXR | Hs.69745; ADXR; ferredoxin reductase | |
| FECH | ferrochelatase (protoporphyria); Hs.26 | |
| FECHP | ferrochelatase pseudogene | |
| FEN1 | RAD2; flap structure-specific endonuclease 1; FEN-l; RAD2 (S. pombe) | |
| | homolog | |
| FENL1 | flap endonuclease-like 1 | |
| FER | fer (fps/fes related) tyrosine kinase (phosphoprotein NCP94); TYK3 | |
| FGFR1 | “fibroblast growth factor receptor 1 (fins-related tyrosine kinase 2, Pfeiffer | |
| | syndrome); Hs.99988; H2; H3; H4; H5; CEK; FLG; FLT2; BFGFR; N- | |
| | SAM; Hs.748” | |
| FGFR2 | “fibroblast growth factor receptor 2 (bacteria-expressed kinase, | |
| | keratinocyte growth factor receptor, craniofacial dysostosis 1, Crouzon | |
| | syndrome, Pfeiffer syndrome, Jackson-Weiss syndrome); Hs.82775; BEK; | |
| | JWS; CEK3; KGFR; TK14; TK25; ECT1; CFD1; K-SAM” | |
| FH | Hs.75 653; fumarate hydratase | |
| FIC1 | “BRIC; PFICl; PFIC; benign recurrent intrahepatic cholestasis; | |
| | progressive familial intrahepatic cholestasis 1, Byler disease; familial | |
| | intrahepatic cholestasis 1” | |
| FLR | flavin reductase (NADPH) | |
| FLT1 | Hs.96085; FLT; fins-related tyrosine kinase 1 (vascular endothelial growth | |
| | factor/vascular permeability factor receptor) | |
| FLT3 | STK1; fms-related tyrosine kinase 3 | |
| FLT3LG | Hs.428; fms-related tyrosine kinase 3 ligand | |
| FLT4 | fms-related tyrosine kinase 4; Hs.74049; VEGFR3 | |
| FMO1 | Hs.1424; flavin containing monooxygenase 1 | |
| FMO2 | Hs.80876; flavin containing monooxygenase 2 | |
| FMO3 | FMOII; flavin containing monooxygenase 3 | |
| FMO4 | Hs.89763; FMO2; flavin containing monooxygenase 4 | |
| FMO5 | Hs.14286; flavin containing monooxygenase 5 | |
| FNTA | “farnesyltransferase, CAAX box, alpha; FPTA; PGGT1A; Hs.78630” | |
| FNTAL1 | “farnesyltransferase, CAAX box, alpha-like 1” | |
| FNTAL2 | “farnesyltransferase, CAAX box, alpha-like 2” | |
| FNTB | “farnesyltransferase, CAAX box, beta; FPTB; Hs.276” | |
| FNTBL1 | “farnesyltransferase, CAAX box, beta-like 1” | |
| FOLH1 | FOLH; folate hydrolase 1 (prostate-specific membrane antigen); PSM | |
| FOLH2 | FOLHP; folate hydrolase 2; folate hydrolase pseudogene | |
| FPGS | folylpolyglutamate synthase | |
| FPGT | GFPP; fucose-1-phosphate guanylyltransferase | |
| FRK | Hs.89426; fyn-related kinase | |
| FTCD | formiminotransferase cyclodeaminase | |
| FTHFD | formyltetrahydrofolate dehydrogenase | |
| FUCA1 | “fucosidase, alpha-L-1, tissue; Hs.576; FUCA” | |
| FUCA1P | “fucosidase, alpha-L-1, tissue pseudogene” | |
| FUCA2 | “fucosidase, alpha-L-2, plasma” | |
| FUT1 | “Hs.69747; H; fucosyltransferase 1 (alpha (1,2) fucosyltransferase, | |
| | Bombay phenotype included)” | |
| FUT2 | fucosyltransferase 2 (secretor status included); SE | |
| FUT3 | “Hs.92753; LE; fucosyltransferase 3 (galactoside 3(4)-L- | |
| | fucosyltransferase, Lewis blood group included); Hs.2527; Hs.89742; | |
| | Hs.92752” | |
| FUT4 | “Hs.2173; CD15; FCT3A; FUC-TIV; fucosyltransferase 4 (alpha (1,3) | |
| | fucosyltransferase, myeloid-speciflc)” | |
| FUT5 | “Hs.32955; FUC-TV; fucosyltransferase 5 (alpha (1,3) | |
| | fucosyltransferase)” | |
| FUT6 | “fucosyltransferase 6 (alpha (1,3) fucosyltransferase)” | |
| FUT7 | “fucosyltransferase 7 (alpha (1,3) fucosyltransferase)” | |
| FUT8 | “fucosyltransferase 8 (alpha (1,6) fucosyltransferase)” | |
| FUT9 | “fucosyltransferase 9 (alpha (1,3) fucosyltransferase); FUC-TIX” | |
| G3BP | Ras-GTPase-activating rotein SH3-domain-binding rotein | |
| G6PC | “G6PT; glucose-6-phosphatase, catalytic (glycogen storage disease type I, | |
| | von Gierke disease); Hs242; GSD1a” | |
| G6PD | glucose-6-phosphate dehydrogenase; Hs.80206; G6PD1; Hs.1435 | |
| G6PDL | glucose-6-phosphate dehydrogenase-like | |
| G6PR | “glucose-6-phosphatase, regulatory; GSD1aSP” | |
| G6PT1 | “glucose-6-phosphatase, transport (glucose-6-phosphate) protein 1; | |
| | GSD1b” | |
| G6PT2 | “glucose-6-phosphatase, transport (phosphate/pyrophosphate) protein 2; | |
| | GSD1c” | |
| G6PT3 | “glucose-6-phosphatase, transport (glucose) protein 3; GSD1d” | |
| G7P1 | kinase-like protein | |
| GAA | “Hs.1437; glucosidase, alpha; acid (Pompe disease, glycogen | storage |
| | disease type II)” | |
| GAD1 | “Hs.75668; GAD; glutamate decarboxylase 1 (brain, 67 kD)” | |
| GAD2 | “Hs.89662 glutamate decarboxylase 2 (pancreatic islets and brain, 65 kD); | |
| | Hs.1668” | |
| GAD3 | glutamate decarboxylase 3 | |
| GAK | cyclin G associated kinase | |
| GALC | galactosylceramidase (Krabbe disease); Hs.273 | |
| GALE | “galactose-4-epimerase, UDP-” | |
| GALGT | “UDP-N-acetyl-alpha-D-galactosamine: (N-acetylneuraminyl)- | |
| | galactosylglucosylceramide N-acetylgalactosaminyltransferase (GalNAc- | |
| | T); beta 1,4GalNAc-T” | |
| GALK1 | GALK; galactokinase 1 | |
| GALK2 | Hs.99935; GK2; galactokinase 2 | |
| GALNS | “GAS; GALNAC6S; galactosamine (N-acetyl)-6-sulfate sulfatase | |
| | (Morquio syndrome, mucopolysaccharidosis type IVA)” | |
| GALNT1 | UDP-N-acetyl-alpha-D-galactosamine:polypeptide N- | |
| | acetylgalactosaminyltransferase 1 (GalNAc-T1) | |
| GALNT2 | UDP-N-acetyl-alpha-D-galactosamine:polypeptide N- | |
| | acetylgalactosaminyltransferase 2 (GalNAc-T2) | |
| GALNT3 | UDP-N-acetyl-a1pha-D-galactosamine:polypeptide N- | |
| | acetylgalactosaminyltransferase 3 (Ga1NAc-T3) | |
| GALNT4 | GALNAC-T4; UDP-N-acetyl-alpha-D-galactosamine:polypeptide N- | |
| | acetylgalactosaminyltransferase 4 (GalNAc-T4) | |
| GALNT5 | GALNAC-T5; UDP-N-acetyl-alpha-D-galactosamine:polypeptide N- | |
| | acetylgalactosaminyltransferase 5 (GalNAc-T5) | |
| GALNT6 | GALNAC-T6; UDP-N-acetyl-alpha-D-galactosamine:polypeptide N- | |
| | acetylgalactosaminyltransferase 6 (GalNAc-T6) | |
| GALNT7 | UDP-N-acetyl-alpha-D-galactosamine:polypeptide | N- |
| | acetylgalactosaminyltransferase 7 (GalNAc-T7) | |
| GALT | Hs.75641; galactose-1-phosphate uridylyltransferase; Hs.56311 | |
| GAMT | guanidinoacetate N-methyltransferase | |
| GANAB | “glucosidase, alpha; neutral AB” | |
| GANC | “glucosidase, alpha; neutral C” | |
| GAPD | Hs.74456; glyceraldehyde-3-phosphate dehydrogenase; GAPDH; G3PDH | |
| GAPDL1 | glyceraldehyde-3-phosphate dehydrogenase-like 1 | |
| GAPDL10 | glyceraldehyde-3-phosphate dehydrogenase-like 10 | |
| GAPDL11 | glyceraldehyde-3-phosphate dehydrogenase-like 11 | |
| GAPDL12 | glyceraldehyde-3-phosphate dehydrogenase-like 12 | |
| GAPDL13 | glyceraldehyde-3-phosphate dehydrogenase-like 13 | |
| GAPDL14 | glyceraldehyde-3-phosphate dehydrogenase-like 14 | |
| GAPDL15 | glyceraldehyde-3-phosphate dehydrogenase-like 15 | |
| GAPDL16 | glyceraldehyde-3-phosphate dehydrogenase-like 16 | |
| GAPDL17 | glyceraldehyde-3-phosphate dehydrogenase-like 17 | |
| GAPDL2 | glyceraldehyde-3-phosphate dehydrogenase-like 2 | |
| GAPDL3 | glyceraldehyde-3-phosphate dehydrogenase-like 3 | |
| GAPDL4 | glyceraldehyde-3-phosphate dehydrogenase-like 4 | |
| GAPDL5 | glyceraldehyde-3-phosphate dehydrogenase-like 5 | |
| GAPDL6 | glyceraldehyde-3-phosphate dehydrogenase-like 6 | |
| GAPDL7 | glyceraldehyde-3-phosphate dehydrogenase-like 7 | |
| GAPDL8 | glyceraldehyde-3-phosphate dehydrogenase-like 8 | |
| GAPDL9 | glyceraldehyde-3-phosphate dehydrogenase-like 9 | |
| GAPDP1 | glyceraldehyde-3-phosphate dehydrogenase pseudogene 1 | |
| GAPDP14 | glyceraldehyde-3-phosphate dehydrogenase pseudogene 14 | |
| GAPL | GTPase activatin protein-like | |
| GARS | Hs.75280; GlyRS; glycyl-tRNA synthetase | |
| GART | “phosphorlbosyiglycinamide formyltransferase, | |
| | phosphoribosylglycinamide synthetase, phosphoribosylaminoimidazole | |
| | synthetase; Hs. 82285; PGFT; PRGS” | |
| GAT | putative glycine-N-acyltransferase | |
| GATM | glycine amidinotransferase (L-arginine:glycine amidinotransferase) | |
| GBA | “glucosidase, beta; acid (includes glucosylceramidase); Hs.80377; GLUC” | |
| GBAP | “glucosidase, beta; acid, pseudogene” | |
| GBE1 | “Hs.1691; glucan (1,4-alpha-), branching enzyme 1 (glycogen branching | |
| | enzyme, Andersen disease, glycogen storage disease type IV)” | |
| GCAT | glycine C-acetyltransferase (2-amino-3-ketobutyrate coenzyme A ligase); | |
| | KBL | |
| GCDH | Hs.63773; glutaryl-Coenzyme A dehydrogenase | |
| GCH1 | GTP cyclohydrolase 1 (dopa-responsive dystonia); Hs.103987; GCH; | |
| | DYT5; GTPCH1; Hs.86724 | |
| GCHFR | GTP cyclohydrolase I feedback regulatory protein; p35; GFRP | |
| GCK | “glucokinase (hexokinase 4, maturity onset diabetes of the young 2); | |
| | Hs.1270; GK; GLK; HK4; NIDDM; MODY2” | |
| GCKR | Hs.89771; glucokinase (hexokinase 4) regulatory protein | |
| GCLC | “GLCLC; glutamate-cysteine ligase, catalytic subunit; Hs.1673; GCS; | |
| | GLCL; glutamate-cysteine ligase (gamma-glutamylcysteine synthetase), | |
| | catalytic (72.8 kD)” | |
| GCLM | “GLCLR; glutamate-cysteine ligase, modifier subunit; Hs.89709; | |
| | glutamate-cysteine ligase (gamma-glutamylcysteine synthetase), | |
| | regulatory (30.8 kD)” | |
| GCNT1 | “Hs.781; C2GNT; NAGCT2; NACGT2; glucosaminyl (N-acetyl) | |
| | transferase 1, core 2 (beta-1,6-N-acetylglucosaminyltransferase); | |
| | C2GnTL; C2GnT-L” | |
| GCNT2 | “Hs.934; IGNT; NAGCT1; NACGT1; glucosaminyl (N-acetyl) | |
| | transferase 2, I-branching enzyme” | |
| GCNT3 | “glucosaminyl (N-acetyl) transferase 3, mucin type; C2GNT-M; | |
| | C2GnTM” | |
| GCS1 | GCS 1-PEN; Glucosidase I | |
| GCTG | gamma-glutamylcyclotransferase | |
| GDA | guanine deaminase | |
| GDH | glucose dehydrogenase | |
| GFPT1 | GFPT; Hs.1674; GFAT; glutamine-fructose-6-phosphate transaminase; | |
| | GFA | |
| GFPT2 | glutamine-fructose-6-phosphate transaminase 2; GFAT2 | |
| GGCX | Hs.77719; gamma-glutamyl carboxylase | |
| GGH | “GH; gamma-glutamyl hydrolase (conjugase, folylpolygammaglutamyl | |
| | hydrolase)” | |
| GGPS1 | GGPPS; geranylgeranyl diphosphate synthase 1 | |
| GGT1 | D22S672; D22S732; GGT; gamma-glutamyltransferase 1 | |
| GGT2 | Hs.56312; GGT; gamma-glutamyltransferase 2 | |
| GGT3 | gamma-glutamyltransferase 3 | |
| GGTA1 | “GGTA; GLYT2; glycoprotein, alpha-galactosyltransferase 1” | |
| GGTA1P | “GLYT3; glycoprotein, alpha-galactosyltransferase 1 pseudogene” | |
| GGTL1 | gamma-glutamyltransferase-like 1 | |
| GGTL2 | gamma-glutamyltransferase-like 2 | |
| GGTL3 | gamma-glutamyltransferase-like 3 | |
| GGTLA1 | GGT-REL; gamma-glutamyltransferase-like activity 1 | |
| GK | Hs.1466; glycerol kinase deficiency | |
| GKP1 | glycerol kinase pseudogene 1 | |
| GKP2 | Hs.2692; glycerol kinase pseudogene 2 | |
| GKP3 | glycerol kinase pseudogene 3 | |
| GKP4 | glycerol kinase pseudogene 4 | |
| GKP5 | glycerol kinase pseudogene 5 | |
| GLA | “GALA; galactosidase, alpha” | |
| GLB1 | “galactosidase, beta 1; Hs.79222” | |
| GLDC | “Hs.27; glycine dehydrogenase (decarboxylating; glycine decarboxylase, | |
| | glycine cleavage system protein P)” | |
| GLDCP | glycine dehydrogenase (decarboxylase) pseudogene | |
| GLO1 | Hs.75207; glyoxalase I | |
| GLRA1 | “STHE; glycine receptor, alpha 1 (startle disease/hyperekplexia, stiff man | |
| | syndrome)” | |
| GLRX | Hs.28988; glutaredoxin (thioltransferase); GRX | |
| GLS | glutaminase | |
| GLUD1 | Hs.77508; GLUP; glutamate dehydrogenase 1 | |
| GLUD2 | Glutamate dehydrogenase-2 | |
| GLUDP1 | glutamate dehydrogenase pseudogene 1 | |
| GLUDP2 | glutamate dehydrogenase pseudogene 2 | |
| GLUDP3 | glutamate dehydrogenase pseudogene 3 | |
| GLUDP4 | glutamate dehydrogenase pseudogene 4 | |
| GLUDP5 | glutamate dehydrogenase pseudogene 5 | |
| GLUL | glutamate-ammonia ligase (glutamine synthase); Hs.1717; GLNS | |
| GLULL1 | glutamate-ammonia ligase (glutamine synthase)-like 1 | |
| GLULL2 | glutamate-ammonia ligase (glutamine synthase)-like 2 | |
| GLULL3 | glutamate-ammonia ligase (glutamine synthase)-like 3 | |
| GLULP | glutamate-ammonia ligase (glutamine synthase) pseudogene | |
| GLYD | glycerate-2-dehydrogeflase | |
| GMDS | “GDP-mannose 4,6-dehydratase” | |
| GMPR | guanine monophosphate reductase | |
| GMPR2 | guanosme monophosphate reductase 2 | |
| GMPS | GMPS-PEN; guanine monophosphate synthetase | |
| GNE | GLCNE; UDP-N-acetylglucosamine-2-epimerase/N-acetylmannosamine | |
| | kinase | |
| GNPAT | DHAPAT; dihydroxyacetone hosphate acyltransferase; DAPAT | |
| GNPTA | glucosamine (UDP-N-acetyl)-lysosomal-enzyme N-acetylglucosamine | |
| | phosphotransferase (mucolipidoses II & III); mucolipidosis II; | |
| | mucolipidosis III | |
| GNS | Hs.2703; glucosamine (N-acetyl)-6-sulfatase (Sanfilippo disease IIID) | |
| GOT1 | “Hs.597; glutamic-oxaloacetic transaminase 1, soluble (aspartate | |
| | aminotransferase 1)” | |
| GOT2 | “Hs.79365; glutamic-oxaloacetic transaminase 2, mitochondrial (aspartate | |
| | aminotransferase 2)” | |
| GOT2L1 | glutamic-oxaloacetic transaminase 2-like 1 | |
| GOT2L2 | glutamic-oxaloacetic transaminase 2-like 2 | |
| GOT2L3 | glutamic-oxaloacetic transaminase 2-like 3 | |
| GPB | “glycerol phosphatase, beta-” | |
| GPD1 | glycerol-3-phosphate dehydrogenase 1 (soluble) | |
| GPD2 | Hs.93201; glycerol-3 -phosphate dehydrogenase 2 (mitochondrial); | |
| | Hs.89720 | |
| GPI | glucose phosphate isomerase; Hs.944 | |
| GPI1 | N-acetylglucosaminyl transferase component Gpi1 | |
| GPLD1 | glycosylphosphatidylinositol specific phospholipase D1 | |
| GPR3 | G protein-coupled receptor 3; ACCA; adenylate cyclase constitutive | |
| | activator | |
| GPRK2L | G protein-coupled receptor kinase 2 (Drosophila)-like; GPRK4 | |
| GPRK5 | GRK5; G protein-coupled receptor kinase 5 | |
| GPRK6 | Hs.76297; GRK6; G protein-coupled receptor kinase 6 | |
| GPRK6P | GPRK6L; G protein-coupled receptor kinase 6 pseudogene; G protein- | |
| | coupled receptor kinase 6-like | |
| GPRK7 | G protein-coupled receptor kinase 7 | |
| GPT | glutamic-pyruvate transaminase (alanine aminotransferase) | |
| GPX1 | Hs.76686; glutathione peroxidase 1 | |
| GPX2 | Hs.2704; GSHPX-GI; glutathione peroxidase 2 (gastrointestinal) | |
| GPX3 | glutathione peroxidase 3 (plasma); Hs.81477 | |
| GPX4 | Hs.2706; glutathione peroxidase 4 (phospholipid hydroperoxidase) | |
| GPX5 | glutathione peroxidase 5 (epididymal androgen-related protein) | |
| GPX6 | glutathione peroxidase 6 (olfactory) | |
| GPX7 | glutathione peroxidase 7 | |
| GPXP1 | GPXL1; glutathione peroxidase pseudogene 1 | |
| GPXP2 | GPXL2; glutathione peroxidase pseudogene 2 | |
| GRHPR | GLXR; glyoxylate reductase/hydroxypyruvate reductase | |
| GSD1B | glycogen-storage disease type 1b | |
| GSD1C | glycogen-storage disease type 1 c | |
| GSE | CD; Gluten-sensitive enteropatby (celiac disease) | |
| GSK1 | glycogen synthase kinase 1 | |
| GSK2 | glycogen synthase kinase 2 | |
| GSK3A | glycogen synthase kinase 3 alpha | |
| GSK3B | glycogen synthase kinase 3 beta | |
| GSPT1 | G1 to S phase transition 1; Hs.2707; GST1 | |
| GSR | glutathione reductase | |
| GSS | Hs.82327; glutathione synthetase | |
| GSTA1 | Hs.100026; H-A; glutathione 5-transferase Al; Hs.89552; Hs.99928 | |
| GSTA2 | glutathione 5-transferase A2; H-A; GST2 | |
| GSTA3 | glutathione S-transferase A3 | |
| GSTA4 | glutathione S-transferase A4 | |
| GSTAP1 | glutathione S-transferase A pseudogene 1 | |
| GSTAP2 | glutathione-S-transferase A pseudogene 2 | |
| GSTM1 | Hs.99859; MU; H-B; GST1; glutathione 5-transferase M1 | |
| GSTM1L | GST1L; glutathione 5-transferase M1-like | |
| GSTM2 | Hs.73974; GST4; glutathione 5-transferase M2 (muscle) | |
| GSTM3 | Hs.2006; GST5; glutathione S-transferase M3 (brain) | |
| GSTM4 | Hs. 105976; glutathione S-transferase M4; Hs.82891 | |
| GSTM5 | Hs.75652; glutathione S-transferase M5 | |
| GSTP1 | FAEES3; glutathione 5-transferase pi; fatty acid ethyl ester synthase III; | |
| | P1; GST3 | |
| GSTPP | glutathione S-transferase pi pseudogene; GST3L; GSTPL | |
| GSTT1 | Hs.77490; glutathione S-transferase theta 1 | |
| GSTT2 | Hs. 1581; glutathione S-transferase theta 2 | |
| GSTTLP28 | P28; glutathione-S-transferase like | |
| GSTZ1 | MAAI; glutathione 5-transferase Zeta 1 (maleylacetoacetate isomerase) | |
| GTA | GGTB1; galactosyltransferase activator | |
| GUCA1A | GUCA1; guanylate cyclase activator 1A (retina); GUCA; GCAP; GCAP1 | |
| GUCA1B | guanylate cyclase activator 1B (retina); GCAP2 | |
| GUCA1C | GCAP3; guanylate cyclase activator 1C | |
| GUCA2A | GUCA2; guanylate cyclase activator 2A (guanylin); Hs.778; STARA | |
| GUCA2B | guanylate cyclase activator 2B; uroguanylin | |
| GUCY1A2 | “GUC1A2; guanylate cyclase 1, soluble, alpha 2; Hs.2685; GC-SA2” | |
| GUCY1A3 | “GUC1A3; guanylate cyclase 1, soluble, alpha 3; GC-SA3” | |
| GUCY1B2 | “guanylate cyclase 1, soluble, beta 2” | |
| GUCY1B3 | “GUC1B3; guanylate cyclase 1, soluble, beta 3; GC-SB3” | |
| GUCY2C | GUC2C; guanylate cyclase 2C (heat stable enterotoxin receptor); guanylyl | |
| | cyclase C; STAR | |
| GUCY2D | “GUC2D; LCA; guanylate cyclase 2D, retina-specific (Leber congenital | |
| | amaurosis 1); Leber congenital amaurosis; Hs.1974; GUC1A4; guanylate | |
| | cyclase 2D, membrane (retina-specific); LCA1; retGC” | |
| GUCY2E | guanylate cyclase 2E; GC-E | |
| GUCY2F | “guanylate cyclase 2F, retinal; GUC2DL; GC-F; RetGC-2; guanylate | |
| | cyclase 2D-like, membrane (retina-specific)” | |
| GUK1 | guanylate kinase 1 | |
| GUK2 | guanylate kinase 2 | |
| GULOP | gulonolactone (L-) oxidase pseudogene; GLO; GULO | |
| GUSB | “Hs.29174; glucuronidase, beta” | |
| GUSM | “glucuronidase, beta (mouse) modifier of” | |
| GYS1 | Hs.772; GYS; glycogen synthase 1 (muscle) | |
| GYS2 | glycogen synthase 2 (liver) | |
| GZMA | “CTLA3; granzyme A (granzyme 1, cytotoxic T-lymphocyte-associated | |
| | serine esterase 3); HFSP” | |
| GZMB | “CTLA1; granzyme B (granzyme 2, cytotoxic T-lymphocyte-associated | |
| | serine esterase 1); CSPB; CCPI; CGL-1; CSP-B” | |
| GZMK | “granzyme K (serine protease, granzyme 3; tryptase II); TRYP2; Hs.3066; | |
| | PRSS; granzyme K (serine protease, granzyme 3); granzyme 3; tryptase | |
| | II” | |
| GZMM | MET1; LMET1; granzyme M (lymphocyte met-ase 1) | |
| H6PD | hexose-6-phosphate dehydrogenase; glucose 1-dehydrogenase | |
| HADH | hydroxyacyl-Coenzyme A dehydrogenase | |
| HADH2 | “hydroxyacyl-Coenzyme A dehydrogenase, type II; ERAB” | |
| HADHA | “hydroxyacyl-Coenzyme A dehydrogenase/3-ketoacyl-Coenzyme A | |
| | thiolase/enoyl-Coenzyme A hydratase (trifunctional protein), alpha | |
| | subunit; Hs.75860; GBP” | |
| HADHAP | hydroxyacyl-Coenzyme A dehydrogenase/3-ketoacyl-Coenzyme A | |
| | thiolase/enoyl-Coenzyme A hydratase pseudogene (gastrin binding | |
| | protein pseudogene) | |
| HADHB | “hydroxyacyl-Coenzyme A dehydrogenase/3-ketoacyl-Coenzyme A | |
| | thiolase/enoyl-Coenzyme A hydratase (trifunctional protein), beta | |
| | subunit” | |
| HADHSC | “L-3-hydroxyacyl-Coenzyme A dehydrogenase, short chain; SCHAD” | |
| HAGH | hydroxyacyl glutathione hydrolase | |
| HAL | Hs.89429; HIS; histidine ammonia-lyase | |
| HAO | “HAO-PEN; 3-hydroxyanthranilate 3,4-dioxygenase” | |
| HAO1 | GOX1; hydroxyacid oxidase (glycolate oxidase) 1; GOX | |
| HARS | Hs.2741; histidyl-tRNA synthetase | |
| HAS1 | HAS; hyaluronan synthase 1 | |
| HAS2 | hyaluronan synthase 2 | |
| HAS3 | hyaluronan synthase 3 | |
| HAT | airway trypsin-like protease | |
| HAT1 | histone acetyltransferase 1 | |
| HBACH | cytosolic acyl coenzyme A thioester hydrolase | |
| HBOA | HBO1; histone acetyltransferase | |
| HCCS | CCHL; holocytochrome c synthase (cytochrome c heme-lyase) | |
| HCK | Hs.89555; JTK9; hemopoietic cell kinase; Hs.77058 | |
| HD | huntingtin (Huntington disease); Hs.79391; IT15 | |
| HDAC1 | “RPD3L1; histone deacetylase 1; HD1; RPD3 (reduced potassium | |
| | dependency, yeast, homolog)-like 1” | |
| HDAC2 | histone deacetylase 2 | |
| HDAC3 | histone deacetylase 3 | |
| HDC | Hs.1481; histidine decarboxylase | |
| HE1 | “NPC2; NP-C2; Niemann-Pick disease, type C; epididymal secretory | |
| | protein (19.5 kD)” | |
| HELLS | “helicase, lymphoid-specific; LSH” | |
| HEP27 | short-chain alcohol dehydrogenase family member | |
| HERA-B | “HERA-A; GTPase, human homolog of E. coli essential cell cycle protein | |
| | Era” | |
| HEXA | hexosaminidase A (alpha polypeptide) | |
| HEXB | Hs.51043; hexosaminidase B (beta polypeptide) | |
| HGD | “AKU; homogentisate 1,2-dioxygenase (homogentisate oxidase); HGG; | |
| | Hs.15113; Alcaptonuria” | |
| HGS | human growth factor-regulated tyrosine kinase substrate; HRS; human | |
| | growth factor-regulated tyrosine kinase substrate | |
| HHCMA56 | putative oxidoreductase | |
| HIBADH | 3-hydroxyisobutyrate dehydrogenase | |
| HINT | PRKCNH1; histidine triad nucleotide-binding protein; protein kinase C | |
| | inhibitor 1; PKCI-1 | |
| HIPK3 | homeodomain-interacting protein kinase 3; PKY; DYRK6 | |
| HK1 | Hs.75276; hexokinase 1 | |
| HK2 | hexokinase 2 | |
| HK2P | hexokinase 2 pseudogene | |
| HK3 | Hs.94397; hexokinase 3 (white cell) | |
| HLCS | holocarboxylase synthetase (biotin-[proprionyl-Coenzyme A-carboxylase | |
| | (ATP-hydrolysing)] ligase); Hs.79375; HCS | |
| HMBS | Hs.82609; UPS; PBGD; hydroxymethylbilane synthase | |
| HMGCL | 3-hydroxy-3-methylglutaryl-Coenzyme A lyase | |
| | (hydroxymethylglutaricaciduria); Hs.831; HL | |
| HMGCR | 3-hydroxy-3-methylglutaryl-Coenzyme A reductase | |
| HMGCS1 | Hs.21808; HMGCS; 3-hydroxy-3-methylglutaryl-Coenzyme A synthase 1 | |
| | (soluble) | |
| HMGCS2 | 3-hydroxy-3-methylglutaryl-Coenzyme A synthase 2 (mitochondrial) | |
| HMOX1 | Hs.75967; heme oxygenase (decycling) 1 | |
| HMOX2 | Hs.83853; HO-2; heme oxygenase (decycling) 2 | |
| HNK-1ST | HNK-1 sulfotransferase | |
| HNMT | histamine N-methyltransferase | |
| HPD | Hs.89831; PPD; 4-hydroxyphenylpyruvate dioxygenase | |
| HPGD | hydroxyprostaglandin dehydrogenase 15-(NAD) | |
| HPN | “hepsin (transmembrane protease, serine 1); Hs.823; TMPRSS1; hepsin” | |
| HPRT1 | Hs.82314; HPRT; HGPRT; hypoxanthine phosphoribosyltransferase 1 | |
| | (Lesch-Nyhan syndrome) | |
| HPRT2 | hypoxanthine phosphoribosyltransferase 2 | |
| HPRTP1 | hypoxanthine phosphoribosyltransferase pseudogene 1 | |
| HPRTP2 | hypoxanthine phosphoribosyltransferase pseudogene 2 | |
| HPRTP3 | hypoxanthine phosphoribosyltransferase pseudogene 3 | |
| HPRTP4 | hypoxanthine phosphoribosyltransferase pseudogene 4 | |
| HPSE | HPA; HSE1; heparanase | |
| HRMT1Ll | “HMT1 (hnRNP methyltransferase, S. cerevisiae)-like 1; PRMT2” | |
| HRMT1L2 | “HMT1 (hnRNP methyltransferase, S. cerevisiae)-like 2; HCP1; PRMT1” | |
| HS3ST1 | heparan sulfate (glucosamine) 3-O-sulfotransferase 1 | |
| HS3ST2 | heparan sulfate (glucosamine) 3-O-sulfotransferase 2 | |
| HS3ST3A1 | heparan sulfate (glucosamine) 3-O-sulfotransferase 3A1 | |
| HS3ST3A2 | heparan sulfate (glucosamine) 3-O-sulfotransferase 3A2 | |
| HS3ST3B1 | heparan sulfate (glucosamine) 3-O-sulfotransferase 3B1 | |
| HS3ST3B2 | heparan sulfate (glucosamine) 3-O-sulfotransferase 3B2 | |
| HS3ST4 | heparan sulfate (glucosamine) 3-O-sulfotransferase 4 | |
| HS6ST | heparan-sulfate 6-sulfotransferase | |
| HSA9947 | putative ATPase | |
| HSCR2 | HSCR; Hirschsprung disease 2 | |
| HSD11B1 | HSD11; HSD11B; hydroxysteroid (11-beta) dehydrogenase 1 | |
| HSD11B2 | hydroxysteroid (11-beta) dehydrogenase 2 | |
| HSD17B1 | HSD17; EDHB17; EDH17B2; hydroxysteroid (17-beta) dehydrogenase 1 | |
| HSD17B2 | Hs.181; hydroxysteroid (17-beta) dehydrogenase 2 | |
| HSD17B3 | Hs.477; hydroxysteroid (17-beta) dehydrogenase 3 | |
| HSD17B4 | hydroxysteroid (17-beta) dehydrogenase 4 | |
| HSD17B5 | hydroxysteroid (17-beta) dehydrogenase 5 | |
| HSD17BP1 | HSD17; EDHB17; EDH17B1; hydroxysteroid (17-beta) dehydrogenase | |
| | pseudogene 1 | |
| HSD3B1 | “Hs.38586; HSDB3; HSD3B; hydroxy-delta-5-steroid dehydrogenase, 3 | |
| | beta- and steroid delta-isomerase 1” | |
| HSD3B2 | “hydroxy-delta-5-steroid dehydrogenase, 3 beta- and steroid delta- | |
| | isomerase 2” | |
| HSD3B3 | “HSD3B3-LSB; hydroxy-delta-5-steroid dehydrogenase, 3 beta- C(27); | |
| | giant cell hepatitis, neonatal” | |
| HSD3BP1 | “hydroxy-delta-5-steroid dehydrogenase, 3 beta, pseudogene 1” | |
| HSD3BP2 | “hydroxy-delta-5-steroid dehydrogenase, 3 beta, pseudogene 2” | |
| HSD3BP3 | “hydroxy-delta-5-steroid dehydrogenase, 3 beta, pseudogene 3” | |
| HSD3BP4 | “hydroxy-delta-5-steroid dehydrogenase, 3 beta, pseudogene 4” | |
| HSD3BP5 | “hydroxy-delta-5-steroid dehydrogenase, 3 beta, pseudogene 5” | |
| HTOR | 5-hydroxytryptamine (serotonin) oxygenase regulator | |
| HTR7 | Hs.73739; 5-hydroxytryptamine (serotonin) receptor 7 (adenylate | |
| | cyclase-coupled) | |
| HU-K5 | lysophospholipase-like | |
| HYAL1 | hyaluronoglucosaminidase 1; LUCA1; HYAL-1 | |
| HYAL2 | LUCA-2; hyaluronoglucosaminidase 2 | |
| HYAL3 | hyaluronoglucosaminidase 3; LUCA-3; LUCA 14; Minna14 | |
| HYL | HYL-PEN; hematopoietic consensus tyrosine-lacking kinase | |
| IARS | Hs.89412; ILRS; isoleucine-tRNA synthetase; Hs.78770 | |
| IBD1 | inflammatory bowel disease 1; Crohn disease | |
| IBGC1 | idiopathic basal ganglia calcification 1; BGCI; IBGC; Fahr disease | |
| ICB-1 | basement membrane-induced gene | |
| IDH1 | “isocitrate dehydrogenase 1 (NADP+), soluble” | |
| IDH2 | “Hs.105969; isocitrate dehydrogenase 2 (NADP+), mitochondrial” | |
| IDH3A | isocitrate dehydrogenase 3 (NAD+) alpha | |
| IDH3B | isocitrate dehydrogenase 3 (NAD+) beta | |
| IDH3G | isocitrate dehydrogenase 3 (NAD+) gamma | |
| IDI1 | isopentenyl diphosphate delta isomerase | |
| IDO | “Hs.840; indole 2,3-dioxygenase” | |
| IDS | iduronate 2-sulfatase (Hunter syndrome); Hs.79285; SIDS | |
| IDSP1 | IDS2; iduronate 2-sulfatase pseudogene 1 | |
| IDUA | “iduronidase, alpha-L-; Hs.89560” | |
| IKBKAP | “inhibitor of kappa light polypeptide gene enhancer in B-cells, kinase | |
| | complex-associated protein; IKAP” | |
| IKBKB | “inhibitor of kappa light polypeptide gene enhancer in B-cells, kinase | |
| | beta; IKK2; NFKBIKB; IKK-beta” | |
| IKBKG | “inhibitor of kappa light polypeptide gene enhancer in B-cells, kinase | |
| | gamma; NEMO; IKK-gamma” | |
| IL17 | CTLA8; interleukin 17 (cytotoxic T-lymphocyte-associated serine | |
| | esterase 8); Hs.41724 | |
| ILF3 | “interleukin enhancer binding factor 3, 90 kD; M-phase phosphoprotein | |
| | 4; NF90; MPP4; DRBP76; NFAR-1; MPHOSPH4; MMP4” | |
| ILK | integrin-linked kinase; Hs.6196 | |
| ILVBL | AHAS; ILV2H; ilvB (bacterial acetolactate synthase)-like | |
| IMPA1 | IMPA; inositol(myo)-1 (or 4)-monophosphatase 1 | |
| IMPA2 | inositol(myo)-1 (or 4)-monophosphatase 2 | |
| IMPDH1 | Hs.850; IMP (inosine monophosphate) dehydrogenase 1; sWSS2608 | |
| IMPDH2 | Hs.75432; IMP (inosine monophosphate) dehydrogenase 2 | |
| IMPDHL1 | IMP (inosine monophosphate) dehydrogenase-like 1 | |
| INDO | “IDO; indoleamine-pyrrole 2,3 dioxygenase” | |
| INMT | ndolethylamine N-methyltransferase; thioester 5-methyltransferase-like; | |
| | indolethylamine N-methyltransferase | |
| INPP1 | inositol polyphosphate-1-phosphatase; Hs.32309 | |
| INPP3 | inositol polyphosphate-3-phosphatase | |
| INPP4A | INPP4; inositol polyphosphate-4-phosphatase | |
| INPP4B | “inositol polyphosphate-4-phosphatase, type II, 105 kD” | |
| INPP5A | “inositol trisphosphate-5-phosphatase, 40 kD; inositol polyphosphate-5- | |
| | phosphatase, 40 kD” | |
| INPP5B | “inositol polyphosphate-5-phosphatase, 75 kD” | |
| INPP5C | “inositol polyphosphate-5-phosphatase, 120 kD” | |
| INPP5D | “inositol polyphosphate-5-phosphatase, 145 kD; SHIP; hp51CN” | |
| INPPL1 | Hs.75339; inositol polyphosphate phosphatase-like 1; SHIP2 | |
| IQGAP2 | IQ motif containing GTPase activating protein 2 | |
| IRAK-M | interleukin-1 receptor-associated kinase M | |
| IRAK1 | interleukin-1 receptor-associated kinase; IRAK; Pelle (Drosophila) | |
| | homolog; pelle | |
| IRAK2 | interleukin-1 receptor-associated kinase 2; IRAK-2 | |
| ITK | IL2-inducible T-cell kinase; EMT; T-celI-specific tyrosine kinase; | |
| | homolog of mouse T-celI itk/tsk tyrosine kinase; PSCTK2 | |
| ITPA | inosine triphosphatase (nucleoside triphosphate pyrophosphatase) | |
| ITPK1 | “inositol 1,3,4-trisphosphate 5/6 kinase” | |
| ITPKA | “Hs.2722; inositol 1,4,5-trisphosphate 3-kinase A” | |
| ITPKB | “Hs.78877; inositol 1,4,5-trisphosphate 3-kinase B” | |
| IVD | Hs.77510; isovaleryl Coenzyme A dehydrogenase | |
| JAK1 | JAK1A; Janus kinase 1 (a protein tyrosine kinase) | |
| JAK2 | Janus kinase 2 (a protein tyrosine kinase) | |
| JAK3 | “Hs.99877; L-JAK; Janus kinase 3 (a protein tyrosine kinase, leukocyte)” | |
| JTK5A | JTK5A protein tyrosine kinase | |
| JTK5B | JTK5B protein tyrosine kinase | |
| KAPPA | “Kappa transcript, coding region similar to kinases” | |
| KAR | Aromatic alpha-keto acid reductase | |
| KARS | lysyl-tRNA synthetase | |
| KATII | kynurenine aminotransferase II | |
| KATNA1 | katanin p60 (ATPase-containing) subunit A1 | |
| KDR | kinase insert domain receptor (a type III receptor tyrosine kinase); | |
| | Hs.12337; FLK1; VEGFR2 | |
| KHK | ketohexokinase (fructokinase); Hs.81454 | |
| KIAA0566 | “ATP#; ATPase type IV, phospholipid transporting (P-type) (putative)” | |
| KIAA0611 | “ATP#; ATPase type IV, phospholipid-transporting (P-type), (putative)” | |
| KIAA0660 | G3BP2; Ras-GTPase activating protein SH3 domain-binding protein 2 | |
| KIAA0901 | HDAC6; histone deacetylase 6 | |
| KIAA0928 | helicase-moi | |
| KIP2 | DNA-dependent protein kinase catalytic subunit-interacting protein 2 | |
| KLK6 | “PRSS9; kallikrein 6 (neurosin, zyme); protease, serine, 9 (neurosin); | |
| | protease M” | |
| KLK7 | “PRSS6; kallikrein 7 (chymotryptic, stratum corneum); SCCE; protease, | |
| | serine, 6 (chymotryptic, stratum corneum)” | |
| KMO | kynurenine 3-monooxygenase (kynurenine 3-hydroxylase) | |
| KNPEP | lysyl aminopeptidase (aminopeptidase Co) | |
| KSR | KSR1; kinase suppressor of ras | |
| KWE | keratolytic winter erythema (Oudtshorn skin disease) | |
| KYN | KYN-PEN; kynureninase | |
| KYNU | kynureninase (L-kynurenine hydrolase) | |
| LAP70 | “apyrase lysosomal” | |
| LARGE | like-glycosyltransferase; KIAA0609 | |
| LARS | leucyl-tRNA synthetase | |
| LAS | lipoic acid synthetase | |
| LCAT | Hs.23513; lecithin-cholesterol acyltransferase; Norum disease; fish-eye | |
| | disease | |
| LCB2 | “KIAA0526; serine palmitoyltransferase, subunit II" | |
| LCK | Hs.1765; lymphocyte-specific protein tyrosine kinase | |
| LCT | Hs.2251; lactase | |
| LDHA | Hs.2795; lactate dehydrogenase A | |
| LDHAL1 | lactate dehydrogenase A-like 1 | |
| LDHAL2 | lactate dehydrogenase A-like 2 | |
| LDHAL3 | lactate dehydrogenase A-like 3 | |
| LDHAL4 | lactate dehydrogenase A-like 4 | |
| LDHAL5 | lactate dehydrogenase A-like 5 | |
| LDHB | Hs.74545; lactate dehydrogenase B | |
| LDHBL1 | lactate dehydrogenase B-like 1 | |
| LDHBP | LDHBL2; lactate dehydrogenase B pseudogene | |
| LDHC | Hs.99881; lactate dehydrogenase C; Hs.511 | |
| LIG1 | “Hs.1770; ligase I, DNA, ATP-dependent” | |
| LIG2 | “ligase II, DNA, ATP-dependent” | |
| LIG3 | “Hs.100299; ligase III, DNA, ATP-dependent” | |
| LIG4 | “ligase IV, DNA, ATP-dependent” | |
| LIM | ENH; LIM protein (similar to rat rotein kinase C-binding enigma) | |
| LIMK1 | LIMK; LIM domain kinase 1; Hs.36566; LIM motif-containing protein | |
| LIMK2 | LIM domain kinase 2 | |
| LIPA | “Hs.85226, lipase A, lysosomal acid, cholesterol esterase (Wolman | |
| | disease)” | |
| LIPB | “lipase B, lysosomal acid” | |
| LIPC | “Hs.9994; lipase, hepatic; HL” | |
| LIPE | “lipase, hormone-sensitive; HSL” | |
| LIPF | “HGL; HLAL; lipase, gastric” | |
| LIPG | “EL; EDL; lipase, endothelial” | |
| LKR/SDH | lysine-ketoglutarate reductase /saccharopine dehydrogenase | |
| LNPEP | leucyl/cystinyl aminopeptidase (oxytocinase); CAP; PLAP | |
| LOAD | late-onset Alzheimer disease susceptibility | |
| LON | “LON-PEN; Lon, ATP-dependent protease (homolog of bacterial Lon)” | |
| LOX | Hs.79234; lysyl oxidase | |
| LOXL1 | LOXL; lysyl oxidase-like 1 | |
| LOXL2 | lysyl oxidase-like 2; WS9-14 | |
| LPAAT- | lysophosphatidic acid acyltransferase beta | |
| BETA | | |
| LPL | lipoprotein lipase; Hs.83122; LIPD | |
| LPO | lactoperoxidase; SPO; salivary peroxidase | |
| LRAT | lecithin retinol acyltransferase (phosphatidylcholine-retinol O- | |
| | acyltransferase) | |
| LSFC | “Leigh syndrome, French-Canadian type (cytochrome oxidase | |
| | deficiency)” | |
| LSK | LSK-PEN; leukocyte carboxyl-terminal src kinase related gene | |
| LSS | “lanosterol synthase (2,3-oxidosqualene-lanosterol cyclase); OSC” | |
| LTA4H | Hs.81118; leukotriene A4 hydrolase | |
| LTB4HD | LTB4HD-PEN; leukotriene B4 12-hydroxydehydrogenase | |
| LTC4S | Hs.456; leukotriene C4 synthase | |
| LTK | Hs.210; TYK1; leukocyte tyrosine kinase | |
| LYPLA1 | lysophospholipase I; LPL1; APT-1; hLysoPLA | |
| LYPLA2 | lysophospholipase II; APT-2 | |
| MACS | “Hs.75607; 80K-L; MARCKS; myristoylated alanine-rich protein kinase | |
| | C substrate (MARCKS, 80K-L)” | |
| MACSL1 | “myristoylated alanine-rich protein kinase C substrate (MARCKS, 80K- | |
| | L)-like 1” | |
| MAK | male germ cell-associated kinase | |
| MAN1A1 | “mannosidase, alpha, class 1A, member 1” | |
| MAN1A2 | “MAN1B; mannosidase, alpha, class 1A, member 2” | |
| MAN1B1 | “MANA-ER; mannosidase, alpha, class 1B, member 1” | |
| MAN2A1 | “MANA2; Hs.75296; mannosidase, alpha type II; Hs.32965” | |
| MAN2A2 | “mannosidase, alpha, class 2A, member 2; mannosidase, alpha | |
| | type II-X; MANA2X” | |
| MAN2B1 | “MANB; Hs.89432; mannosidase, alpha B, lysosomal; LAMAN; Hs.375” | |
| MAN2C1 | “MANA1; MANA; mannosidase, alpha A, cytoplasmic” | |
| MANBA | “mannosidase, beta A, lysosomal” | |
| MANBB | “mannosidase, beta B, soluble” | |
| MAOA | Hs.1782; monoamine oxidase A | |
| MAOB | Hs.82163; monoamine oxidase B | |
| MAP2K1 | “PRKMK1; MEK1; MAPKK1; protein kinase, mitogen-activated, kinase | |
| | 1 (MAP kinase kinase 1); MKK1” | |
| MAP2K2 | “PRKMK2; MEK2; protein kinase, mitogen-activated, kinase 2, p45 | |
| | (MAP kinase kinase 2)” | |
| MAP2K3 | “PRKMK3; protein kinase, mitogen-activated, kinase 3 (MAP kinase | |
| | kinase 3); MEK3; MKK3” | |
| MAP2K4 | SERK1; SAPK/Erk kinase 1; MEK4; JNKK1; PRKMK4 | |
| MAP2K5 | “PRKMK5; protein kinase, mitogen-activated, kinase 5 (MAP kinase | |
| | kinase 5); MEK5” | |
| MAP2K6 | “PRKMK6; protein kinase, mitogen-activated, kinase 6 (MAP kinase | |
| | kinase 6); MEK6; MKK6; SAPKK3” | |
| MAP2K7 | “PRKMK7; MKK7; protein kinase, mitogen-activated, kinase 7 (MAP | |
| | kinase kinase 7); Jnkk2; MAPKK7” | |
| MAP3K1 | MEKK1; MAP/ERK kinase kinase 1; MEKK; MAPKKK1 | |
| MAP3K10 | “MLK2; mitogen-activated protein kinase kinase kinase 10; mixed | |
| | lineage kinase 2 (tyr and ser/thr specificity); serine/threonine | |
| | kinase, non-receptor type; MST” | |
| MAP3K11 | MLK3; mixed lineage kinase 3; PTK1; SPRK; MLK-3 | |
| MAP3K12 | ZPK; zipper (leucine) protein kinase | |
| MAP3K13 | LZK; mitogen-activated protein kinase kinase kinase 13 | |
| MAP3K2 | MEKK2; MAP3K2-PENDING; mitogen-activated protein kinase kinase | |
| | kinase 2 | |
| MAP3K3 | MEKK3; MAP/ERK kinase kinase 3; MAPKKK3 | |
| MAP3K4 | MEKK4; MAP/ERK kinase kinase 4; MTK1; MAPKKK4 | |
| MAP3K5 | MEKK5; MAP/ERK kinase kinase 5; ASK1; MAPKKK5 | |
| MAP3K6 | mitogen-activated protein kinase kinase kinase 6; MAPKKK6 | |
| MAP3K7 | TAK1; transforming growth factor beta-activated kinase 1 | |
| MAP3K9 | MLK1; mixed lineage kinase 1 (tyr and ser/thr specificity); PRKE1 | |
| MAP4K1 | HPK1; mitogen-activated protein kinase kinase kinase kinase 1 | |
| MAP4K2 | RAB8IP; Rab8 interacting protein (GC kinase); GCK; BL44 | |
| MAP4K3 | GLK; RAB8IPL1; mitogen-activated protein kinase kinase kinase kinase 3 | |
| MAP4K4 | HGK; NIX; KIAA0687; mitogen-activated protein kinase kinase kinase | |
| | kinase 4 | |
| MAP4K5 | mitogen-activated protein kinase kinase kinase kinase 5; KHS; | |
| | KHS-PEN; kinase homologous to SPS1/STE20; KHS1 | |
| MAPK1 | “PRKM1; mitogen-activated protein kinase 1; PRKM2; protein kinase, | |
| | mitogen-activated 1 (MAP kinase 1; p40, p41); ERK; ERK2; MAPK2; | |
| | p4lmapk; p38” | |
| MAPK11 | PRKM11; mitogen-activated protein kinase 11; protein kinase mitogen- | |
| | activated 11; SAPK2; p38-2; p38Beta | |
| MAPK12 | SAPK3; stress-activated protein kinase 3; ERK6; PRKM12; p38gamma | |
| MAPK13 | PRKM13; protein kinase mitogen-activated 13; SAPK4; p38delta | |
| MAPK14 | CSBP1; CSBP2; cytokine suppressive anti-inflammatory drug binding | |
| | protein 2 (p38 MAP kinase); CSPB1; cytokine suppressive anti- | |
| | inflammatory drug binding protein 1; PRKM14; p38; Mxi2; PRKM15 | |
| MAPK3 | “PRKM3; protein kinase, mitogen-activated 3 (MAP kinase 3; p44); | |
| | ERK1; p44mapk; p44erk1” | |
| MAPK4 | “PRKM4; protein kinase, mitogen-activated 4 (MAP kinase 4; p63); | |
| | Erk3-related; ERK3” | |
| MAPK6 | “PRKM6; protein kinase, mitogen-activated 6 (extracellular signal- | |
| | regulated kinase, p97); protein kinase, mitogen-activated 5 (extracellular | |
| | signal-regulated kinase, p97); ERK3; p97MAPK” | |
| MAPK7 | PRKM7; mitogen-activated protein kinase 7; BMK1; ERK5 | |
| MAPKAPK2 | mitogen-activated protein kinase-activated protein kinase 2 | |
| MAPKAPK3 | MAPKAP; 3pK; mitogen-activated protein kinase-activated protein kinase | |
| MAPKAPK5 | mitogen-activated protein kinase-activated protein kinase 5; PRAK | |
| MARK1 | MAP/microtubule affinity-regulating kinase 1 | |
| MARK3 | MAP/microtubule affinity-regulatin kinase 3; KP78 | |
| MARS | methionine-tRNA synthetase | |
| MASP1 | “PRSS5; MASP; protease, serine, 5 (mannose-binding protein- | |
| | associated)” | |
| MAT1A | “MAT; methionine adenosyltransferase I, alpha; SAMS; MATA1; | |
| | SAMS1” | |
| MAT2A | “methionine adenosyltransferase II, alpha; SAMS2; MATA2; MATII” | |
| MATK | Hs.274; megakaryocyte-associated tyrosine kinase | |
| MCCC1 | methylcrotonoyl-Coenzyme A carboxylase 1 (alpha) | |
| MCCC2 | methylcrotonoyl-Coenzyme A carboxylase 2 (beta) | |
| MCD | malonyl coenzyme A decarboxylase | |
| MCKD1 | medullary cystic kidney disease I (autosomal dominant); ADMCKD; | |
| | MCD; MCKD; ADMCKD1 | |
| MCKD2 | ADMCKD2; medullary cystic kidney disease 2 (autosomal dominant) | |
| MDH1 | “Hs.75375; malate dehydrogenase 1, NAD (soluble)” | |
| MDH2 | “malate dehydrogenase 2, NAD (mitochondrial)” | |
| ME78 | ME78-PEN; Metallo-endopeptidase(78KDa)(cleaves a beta-APP substrate | |
| MEB | muscle-eye-brain disease | |
| MED6 | “RNA polymerase II transcriptional regulation mediator (Med6, S. | |
| | cerevisiae, homolog of)” | |
| MEP1A | “meprin A, alpha (PABA peptide hydrolase); PPHA” | |
| MERTK | “c-mer proto-oncogene tyrosine kinase; MER; MER-PEN; | |
| | protooncogene C-mer (tyrosine kinase expressed in monocytes, | |
| | epithelial, and reproductive tissues); c-mer” | |
| METTL1 | methyltransferase-like 1; C12orfl; YDL201w | |
| MGAM | MG; MGA; maltase-glucoamylase (alpha-glucosidase) | |
| MGAT1 | “mannosyl (alpha-1,3-)-glycoprotein beta-1,2-N- | |
| | acetylglucosaminyltransferase; Hs.82148; GNT-I; MGAT; GLYT1; | |
| | GLCNAC-TI” | |
| MGAT2 | “mannosyl (alpha-1,6-)-glycoprotein beta-1,2-N- | |
| | acetylglucosaminyltransferase; GNT-II” | |
| MGAT3 | “mannosyl (beta-1,4-)-glycoprotein beta-1,4-N- | |
| | acetylglucosaminyltransferase; Hs.112; GNT-III” | |
| MGAT4A | “GNT-IV; GNT-IVA; mannosyl (alpha-1,3 -)-glycoprotein beta-1,4-N- | |
| | acetylglucosaminyltransferase, isoenzyme A” | |
| MGAT4B | “GNT-IV; GNT-IVB; mannosyl (alpha-1,3-)-glycoprotein beta-1,4-N- | |
| | acetylglucosaminyltransferase, isoenzyme B” | |
| MGAT5 | “mannosyl (alpha-1,6-)-glycoprotein beta-1,6-N- | |
| | acetylglucosaminyltransferase; GNT-V” | |
| MGEA5 | MEA5; meningioma expressed antigen 5 (hyaluronidase) | |
| MGMT | O-6-methylguanine-DNA methyltransferase; Hs.1384 | |
| MGST1 | MGST; microsomal glutathione S-transferase 1; MGST-I; GST12 | |
| MGST1L1 | PIG12; MGST-IV; microsomal glutathione S-transferase 1-like 1 | |
| MGST2 | microsomal glutathione S-transferase 2; MGST-II | |
| MGST3 | microsomal glutathione S-transferase 3 | |
| MINPP1 | MIPP; MINPP2; multiple inositol polyphosphate phosphatase 1 | |
| MIPEP | mitochondrial intermediate peptidase; MW; HMIP | |
| MJD | “Machado-Joseph disease (spinocerebellar ataxia 3, olivopontocerebellar | |
| | ataxia 3, autosomal dominant, ataxin 3); ATX3; SCA3; Machado-Joseph | |
| | disease” | |
| MJD4 | MJD4-PEN; Machado-Joseph disease-related 4 | |
| MJDL1 | MJD2; Machado-Joseph disease-like-1 | |
| MKNK1 | MNK1; MAP kinase-interacting serine/threonine kinase 1 | |
| MKP-L | MKP-1 like protein tyrosine phosphatase | |
| MKP5 | dual specificity phosphatase MKP-5 | |
| MLD | membrane fatty acid (lipid) desaturase | |
| MMCP-7- | MMCP-7-LIKE-1; mast cell tryptase | |
| LIKE-2 | | |
| MME | “membrane metallo-endopeptidase (neutral endopeptidase, | |
| | enkephalinase, CALLA, CD10); Hs.1298; CD10; CALLA” | |
| MMP1 | Hs.83169; CLG; matrix metalloproteinase 1 (interstitial collagenase) | |
| MMP10 | Hs.2258; STMY2; matrix metalloproteinase 10 (stromelysin 2) | |
| MMP11 | STMY3; matrix metalloproteinase 11 (stromelysin 3) | |
| MMP12 | matrix metalloproteinase 12 (macrophage elastase); Hs.1695; HME | |
| MMP13 | Hs.2936; CLG3; matrix metalloproteinase 13 (collagenase 3) | |
| MMP14 | matrix metalloproteinase 14 (membrane-inserted); MT1-MMP | |
| MMP15 | matrix metalloproteinase 15 (membrane-inserted); MT2-MMP | |
| MMP16 | matrix metalloproteinase 16 (membrane-inserted); MT3-MMP | |
| MMP17 | matrix metalloproteinase 17 (membrane-inserted); MT4-MMP | |
| MMP19 | MMP18; matrix metalloproteinase 19; matrix metalloproteinase 18; | |
| | RASI-1 | |
| MMP2 | “Hs.80343; CLG4; CLG4A; matrix metalloproteinase 2 (gelatinase A, | |
| | 72 kD gelatinase, 72 kD type IV collagenase); Hs.75399; Hs.75557” | |
| MMP20 | matrix metalloproteinase 20; enamelysin | |
| MMP23A | MIFR; MMP21; MIFR-1; matrix metalloproteinase 23A | |
| MMP23B | MMP22; matrix metalloproteinase 23B | |
| MMP24 | MT5-MMP; matrix metalloproteinase 24 (membrane-inserted) | |
| MMP3 | “Hs.83326; STMY; STMY1; matrix metalloproteinase 3 (stromelysin 1, | |
| | progelatinase); Hs.46450” | |
| MMP7 | “Hs.2256; MPSL1; MMP-7; PUMP-1; matrix metalloproteinase 7 | |
| | (matrilysin, uterine)” | |
| MMP8 | Hs.73862; CLG1; matrix metalloproteinase 8 (neutrophil collagenase) | |
| MMP9 | “CLG4B; matrix metalloproteinase 9 (gelatinase B, 92 kD gelatinase, | |
| | 92 kD type IV collagenase)” | |
| MMPL1 | matrix metalloproteinase-like 1; MMP20; matrix metalloproteinase-like 1 | |
| MMSDH | methylmalonate-semialdehyde dehydrogenase | |
| MOX1 | mitogenic oxidase (pyridine nucleotide-dependent superoxide-generating) | |
| MPG | Hs.79396; MDG; N-methylpurine-DNA glycosylase | |
| MPI | Hs.75694; mannose phosphate isomerase | |
| MPO | myeloperoxidase; Hs.1817 | |
| MPP-1 | M-phase phosphoprotein I | |
| MPP-10 | M phase phosphoprotein 10 (U3 small nucleolar ribonucleoprotein) | |
| MPP-6 | M-phase phosphoprotein 6 | |
| MPP-9 | M phase phosphoprotein 9 | |
| MPST | mercaptopyruvate sulfurtransferase; MST | |
| MSRA | methionine sulfoxide reductase A | |
| MST-3 | STE20-like kinase 3 | |
| MST1R | Hs.2942; RON; macrophage stimulating 1 receptor (c-met-related | |
| | tyrosine kinase) | |
| MTAP | Hs.3245; methylthioadeno sine phosphorylase | |
| MTAPP | MTAPP-PEN; methylthioadensine phosphorylase pseudogene | |
| MTATP6 | ATP synthase 6 | |
| MTATP8 | ATP synthase 8 | |
| MTCO1 | cytocbrome c oxidase I | |
| MTCO2 | cytochrome c oxidase II | |
| MTCO3 | cytochrome c oxidase III | |
| MTHFD1 | “MTHFD; Hs.37791; MTHFC; 5,10-methylenetetrahydrofolate | |
| | dehydrogenase, 5,10-methylenetetrahydrofolate cyclohydrolase, 10- | |
| | formyltetrahydrofolate synthetase; Hs.1793” | |
| MTHFD1P1 | “MTHFDP1; MTHFDL1; 5,10-methylenetetrahydrofolate | |
| | dehydrogenase, 5,10-methylenetetrahydrofolate cyclohydrolase, | |
| | 10-formyltetrahydrofolate synthetase pseudogene 1” | |
| MTHFD2 | “NMDMC; methylene tetrahydrofolate dehydrogenase (NAD+ | |
| | dependent), methenyltetrahydrofolate cyclohydrolase” | |
| MTHFR | “5,10-methylenetetrahydrofolate reductase (NADPH)” | |
| MTHFS | “MTHFS-PEN; 5,10-methenyltetrahydrofolate synthase” | |
| MTND1 | “NADH dehydrogenase, subunit 1 (complex I)” | |
| MTND2 | “NADH dehydrogenase, subunit 2 (complex I)” | |
| MTND3 | “NADH dehydrogenase, subunit 3 (complex I)” | |
| MTND4 | “NADH dehydrogenase, subunit 4 (complex I)” | |
| MTND4L | “NADH dehydrogenase, subunit 4L (complex I)” | |
| MTND5 | “NADH dehydrogenase, subunit 5 (complex I)” | |
| MTND6 | “NADH dehydrogenase, subunit 6 (complex I)” | |
| MTR | 5-methyltetrahydrofolate-homocysteine methyltransferase | |
| MTRF1 | mitochondrial translational release factor 1; MTTRF1; RF1 | |
| MTRR | 5-methyltetrahydrofolate-homocysteine methyltransferase reductase | |
| MUSK | “muscle, skeletal, receptor tyrosine kinase” | |
| MUT | methylmalonyl Coenzyme A mutase | |
| MVD | mevalonate (diphospho) decarboxylase; MPD | |
| MVK | mevalonate kinase (mevalonic aciduria); Hs.75138 | |
| MYHK | “myosin, heavy polypeptide kinase” | |
| MYLK | “myosin, light polypeptide kinase” | |
| MYLKP | “myosin, light polypeptide kinase pseudogene” | |
| MYP1 | “myopia 1 (X-linked, Bornholm eye disease included)” | |
| MYPT1 | “myosin phosphatase, target subunit 1; MBS” | |
| MYPT2 | “myosin phosphatase, target subunit 2” | |
| NAADALAS | NAALADASE2; N-acetylated alpha-linked acidic dipeptidase II | |
| E2 | | |
| NAALADAS | I100; N-acetylated alpha-linked acidic dipeptidase-like; ILEAL | |
| EL | DIPEPTIDYLPEPTIDASE | |
| NAGA | “N-acetylgalactosaminidase, alpha-; D22S674; Hs.75372; GALB” | |
| NAGLU | “N-acetylglucosaminidase, alpha- (Sanfilippo disease IIIB); Hs.50727; | |
| | NAG” | |
| NARS | asparaginyl-tRNA synthetase | |
| NAT1 | AAC1; Hs.89391; arylamide acetylase 1 (N-acetyltransferase 1) | |
| NAT2 | “AAC2; Hs.2; arylamide acetylase 2 (N-acetyltransferase 2, isoniazid | |
| | inactivation)” | |
| NCF1 | “Hs.1583; neutrophil cytosolic factor 1 (47 kD, chronic granulomatous | |
| | disease, autosomal 1); p47phox” | |
| NCF2 | “Hs.949; neutrophil cytosolic factor 2 (65 kD, chronic granulomatous | |
| | disease, autosomal 2); p67phox” | |
| NCK1 | NCK; Hs.54589; non-catalytic region of tyrosine kinase | |
| NDP | Hs.2839; Norrie disease (pseudoglioma) | |
| NDR | “NDR-LSB; serine/threonine kinase, nuclear Dfnb2-related (Drosophila) | |
| | homolog” | |
| NDST1 | HSST; N-deacetylase/N-sulfotransferase (heparan glucosaminyl); heparan | |
| | sulfate-N-deacetylase/N-sulfotransferase; Hs.20894; NST1 | |
| NDST2 | N-deacetylase/N-sulfotransferase (heparan glucosaminyl) 2; NST2; | |
| | HSST2; N-deacetylase/N-sulfotransferase (heparan glucosaminyl) 2 | |
| NDST3 | N-deacetylase/N-sulfotransferase (heparan glucosaminyl) 3 | |
| NDUFA1 | “NADH dehydrogenase (ubiquinone) 1 alpha subcomplex, 1 (7.5 kD, | |
| | MWFE); MWFE” | |
| NDUFA10 | “NADH dehydrogenase (ubiguinone) 1 alpha subcomplex, 10 (42 kD)” | |
| NDUFA2 | “NADH dehydrogenase (ubiquinone) 1 alpha subcomplex, 2 (8 kD, B8); | |
| | B8 | |
| NDUFA3 | “NADH dehydrogenase (ubiquinone) 1 alpha subcomplex, 3 (9 kD, B9); | |
| | B9” | |
| NDUFA4 | “NADH dehydrogenase (ubiquinone) 1 alpha subcomplex, 4 (9 kD, | |
| | MLRQ); MLRQ” | |
| NDUFA5 | “NADH dehydrogenase (ubiquinone) 1 alpha subcomplex, 5 | |
| | (13 kD, B13); B13” | |
| NDUFA5P1 | “NADH dehydrogenase (ubiquinone) 1 alpha subcomplex, 5, | |
| | pseudogene 1” | |
| NDUFA6 | “NADH dehydrogenase (ubiquinone) 1 alpha subcomplex, 6 | |
| | (14 kD, B 14); B14” | |
| NDUFA7 | “NADH dehydrogenase (ubiquinone) 1 alpha subcomplex, 7 (14.5 kD, | |
| | B14.5a); B14.5a” | |
| NDUFA8 | “NADH dehydrogenase (ubiquinone) 1 alpha subcomplex, 8 (19 kD, | |
| | PGIV); PGIV” | |
| NDUFA9 | “NADH dehydrogenase (ubiguinone) 1 alpha subcomplex, 9 (39 kD)” | |
| NDUFAB1 | “NADH dehydrogenase (ubiquinone) 1, alpha/beta suboomplex, 1 | |
| | (8 kD, SDAP); SDAP” | |
| NDUFB1 | “NADH dehydrogenase (ubiquinone) 1 beta subcomplex, 1 (7 kD, MNLL); | |
| | MNLL” | |
| NDUFB10 | “NADH dehydrogenase (ubiquinone) 1 beta subcomplex, 10 (22 kD, | |
| | PDSW); PDSW” | |
| NDUFB2 | “NADH dehydrogenase (ubiquinone) 1 beta subcomplex, 2 (8 kD, | |
| | AGGG); AGGG” | |
| NDUFB3 | “NADH dehydrogenase (ubiquinone) 1 beta subcomplex, 3 (12 kD, | |
| | B12); B12” | |
| NDUFB4 | “NADH dehydrogenase (ubiquinone) 1 beta subcomplex, 4 (15 kD, | |
| | B15); B15” | |
| NDUFB5 | “NADH dehydrogenase (ubiquinone) 1 beta subcomplex, 5 (16 kD, | |
| | SGDH); SGDH” | |
| NDUFB6 | “NADH dehydrogenase (ubiquinone) 1 beta subcomplex, 6 (17 kD, | |
| | B17); B17” | |
| NDUFB7 | “NADH dehydrogenase (ubiquinone) 1 beta subcomplex, 7 (18 kD, | |
| | B18); B18” | |
| NDUFB8 | “NADH dehydrogenase (ubiquinone) 1 beta subcomplex, 8 (19 kD, | |
| | ASHI); ASHI” | |
| NDUFB9 | “B22; NADH dehydrogenase (ubiquinone) 1 beta subcomplex, 9 (22 kD, | |
| | B22); UQOR22” | |
| NDUFC1 | “NADH dehydrogenase (ubiquinone) 1, subcomplex unknown, 1 (6 kD, | |
| | KFYI); KFYI” | |
| NDUFC2 | “NADH dehydrogenase (ubiquinone) 1, subcomplex unknown, 2 | |
| | (14.5 kD, B14.5b); B14.5b” | |
| NDUFS1 | NADH dehydrogenase (ubiquinone) Fe-S protein 1 (75 kD) (NADH- | |
| | coenzyme Q reductase); Hs.8248; NADH-UBIQUINONE | |
| | OXIDOREDUCTASE 75 KD SUBUNIT PRECURSOR | |
| NDUFS2 | NADH dehydrogenase (ubiquinone) Fe-S protein 2 (49 kD) (NADH- | |
| | coenzyme Q reductase) | |
| NDUFS2L | NADH dehydrogenase (ubiquinone) Fe-S protein 2-like (NADH- | |
| | coenzyme Q reductase) | |
| NDUFS3 | NADH dehydrogenase (ubiquinone) Fe-S protein 3 (30 kD) (NADH- | |
| | coenzyme Q reductase) | |
| NDUFS4 | NADH dehydrogenase (ubiquinone) Fe-S protein 4 (18 kD) (NADH- | |
| | coenzyme Q reductase); AQDQ; mitochondrial respiratory chain | |
| | complex I (18-RD subunit) | |
| NDUFS5 | NADH dehydrogenase (ubiquinone) Fe-S protein 5 (15 kD) (NADH- | |
| | coenzyme Q reductase) | |
| NDUFS6 | NADH dehydrogenase (ubiquinone) Fe-S protein 6 (13 kD) (NADH- | |
| | coenzyme Q reductase) | |
| NDUFS7 | NADH dehydrogenase (ubiquinone) Fe-S protein 7 (20 kD) (NADH- | |
| | coenzyme Q reductase); PSST | |
| NDUFS8 | NADH dehydrogenase (ubiquinone) Fe-S protein 8 (23 kD) (NADH- | |
| | coenzyme Q reductase) | |
| NDUFV1 | NADH dehydrogenase (ubiquinone) flavoprotein 1 (51 kD) | |
| NDUFV2 | Hs.51299; NADH dehydrogenase (ubiquinone) flavoprotein 2 (24 kD) | |
| NDUFV2P1 | NADH dehydrogenase (ubiquinone) flavoprotein 2 pseudogene 1 | |
| NDUFV3 | NADH dehydrogenase (ubiquinone) flavoprotein 3 (10 kD) | |
| NEK1 | NIMA (never in mitosis gene a)-related kinase 1 | |
| NEK2 | NIMA (never in mitosis gene a)-related kinase 2; NLK; 1 HSPK21 | |
| NEK3 | NIMA (never in mitosis gene a)-related kinase 3 | |
| NEK4 | NIMA (never in mitosis gene a)-related kinase 4; NLK2 | |
| NEK5 | NIMA (never in mitosis gene a)-related kinase 5; NLK3 | |
| NEK6 | NIMA (never in mitosis gene a)-related kinase 6 | |
| NEU1 | NEU; neuraminidase; sialidase | |
| NEU2 | sialidase 2 (cytosolic sialidase) | |
| NEU3 | neuraminidase 3 (membrane sialidase) | |
| NF1 | “neurofibromin 1 (neurofibromatosis, von Recklinghausen disease, | |
| | Watson disease); Hs.93207; Hs.37170; Hs.89393” | |
| NGAP | ras GTPase activating protein-like | |
| NIFS | cysteine desulfurase | |
| NIK | HS; HSNIK; serine/threonine protein-kinase | |
| NIPSNAP1 | 4-nitrophenyiphosphatase domain and non-neuronal SNAP25-like 1 | |
| NIT1 | nitrilase 1 | |
| NM23-H6 | NME6; IPIA-ALPHA; nucleoside diphosphate kinase type 6 (inhibitor | |
| | of p53-induced apoptosis-alpha) | |
| NME4 | “non-metastatic cells 4, protein expressed in (nucleoside-diphosphate | |
| | kinase); nm23-H4” | |
| NME5 | “non-metastatic cells 5, protein expressed in (nucleoside-diphosphate | |
| | kinase)” | |
| NMOR2 | “Hs.73956; NQ02; NAD(P)H menadione oxidoreductase 2, dioxin- | |
| | inducible” | |
| NMT1 | NMT; N-myristoyltransferase | |
| NMT2 | N-myristoyltransferase 2 | |
| NNMT | nicotinamide N-methyltransferase | |
| NNT | NNT-PEN; nicotinamide nucleotide transhydrogenase | |
| NOD1 | CARD4; caspase recruitment domain 4 | |
| NOS1 | Hs.46752; NOS; nitric oxide synthase 1 (neuronal) | |
| NOS2A | “Hs.946; NOS2; nitric oxide synthase 2A (inducible, hepatocytes)” | |
| NOS2B | nitric oxide synthase 2B | |
| NOS2C | nitric oxide synthase 2C | |
| NOS3 | nitric oxide synthase 3 (endothelial cell); Hs.76983; consitutive | |
| | endothelial nitric oxide synthase; ECNOS | |
| NP | Hs.75514; nucleoside phosphorylase | |
| NPC1 | “NPC; Niemann-Pick disease, type C1” | |
| NPR1 | NPRA; ANPRA; GUC2A; natriuretic peptide receptor Alguanylate | |
| | cyclase A (atrionatriuretic peptide receptor A) | |
| NPR2 | NPRB; ANPRB; GUC2B; natriuretic peptide receptor B/guanylate | |
| | cyclase B (atrionatriuretic peptide receptor B) | |
| NPR2L | homologous to yeast nitrogen permease (candidate tumor suppressor) | |
| NRD1 | nardilysin (N-arginine dibasic convertase) 1; hNRD1; hNRD2 | |
| NRGN | “neurogranin (protein kinase C substrate, RC3); RC3” | |
| NSEP1 | DBPB; nuclease sensitive element binding protein 1 | |
| NSMAF | neutral sphingomyelinase (N-SMase) activation associated factor; FAN | |
| NT3 | 3′ nucleotidase | |
| NT5 | Hs.76856; CD73; 5′ nucleotidase (CD73); Hs.2382 | |
| NT5CP | NT5CP-LSB; cytosolic purine 5′ nucleotidase | |
| NTE | neuropathy target esterase | |
| NTHL1 | nth (E. coli endonuclease III)-like 1; NTH1; OCTS3 | |
| NTRK1 | “TRK; neurotrophic tyrosine kinase, receptor, type 1” | |
| NTRK2 | “TRKB; neurotrophic tyrosine kinase, receptor, type 2” | |
| NTRK3 | “Hs.26776; TRKC; neurotrophic tyrosine kinase, receptor, type 3” | |
| NTRKR1 | neurotrophic tyrosine kinase receptor-related 1; Ror1 | |
| NTRKR2 | neurotrophic tyrosine kinase receptor-related 2; Ror2 | |
| NUDT1 | “MTH1; Hs.388; mutT (E. coli) human homolog (8-oxo-7,8- | |
| | dihydroguanosine triphosphatase)” | |
| NUDT2 | “APAH1; Ap4A hydrolase 1 (diadenosine 5′,5″″′-P1,P4-tetraphosphate | |
| | pyrophosphohydrolase)” | |
| NY-CO-9 | HDAC5; histone deacetylase 5; KIAA0600 | |
| OAS1 | “OIAS; ′,5′-oligoadenylate synthetase; Hs.82396; IFI-4” | |
| OAS2 | 2′-5′oligoaden late synthetase 2 | |
| OAS3 | 2′-5′oligoadenylate synthetase 3 | |
| OASL | TRIP14; 2′-5′oligoadenylate synthetase-like | |
| OAT | Hs.75485; ornithine aminotransferase (gyrate atrophy) | |
| OATL1 | ornithine aminotransferase-like 1 | |
| OATL2 | ornithine aminotransferase-like 2 | |
| OATL3 | ornithine aminotransferase-like 3 | |
| OAZ1 | OAZ; ornithine decarboxylase antizyme | |
| OAZ2 | ornithine decarboxylase antizyme 2 | |
| OC90 | PLA2L; otoconin 90; phospholipase A2-like | |
| ODC1 | Hs.75212; ornithine decarboxylase 1 | |
| ODCP | ODC2; ornithine decarboxylase pseudogene | |
| OED | Oregon eye disease | |
| OGDH | Hs.75533; oxoglutarate dehydrogenase (lipoamide) | |
| OGG1 | 8-oxoguanine DNA glycosylase | |
| OGT | O-GLCNAC; O-linked N-acetylglucosamine (GlcNAc) transferase | |
| | (UDP-N-acetylglucosamine:polypeptide-N-acetylglucosaminyl | |
| | transferase) | |
| OTC | Hs.1842; ornithine carbamoyltransferase | |
| OVD1A | 2-oxoisovalerate dehydrogenase (lipoamide) | |
| OXA1L | oxidase (cytochrome c) assembly 1-like | |
| OXCT | 3-oxoacid CoA transferase; SCOT | |
| P-CIP1 | peptidylglycine alpha-amidating monooxygenase COOH-terminal | |
| | interactor protein-1 | |
| P11 | PP11; placental protein 11 (serine proteinase) | |
| P4HA1 | “P4HA; Hs.89513; procollagen-proline, 2-oxogilutarate 4-dioxygenase | |
| | (proline 4-hydroxylase), alpha polypeptide; Hs.76768” | |
| P4HA2 | “procollagen-proline, 2-oxoglutarate 4-dioxygenase (proline 4- | |
| | hydroxylase), alpha polypeptide II” | |
| P4HB | “ERBA2L; procollagen-proline, 2-oxoglutarate 4-dioxygenase (proline 4- | |
| | hydroxylase), beta polypeptide (protein disulfide isomerase; thyroid | |
| | hormone binding protein p55); Hs.89698; PO4DB; v-erb-a avian | |
| | erythroblastic leukemia viral oncogene homolog 2-like; Hs.75655” | |
| P4HBR | “P4HBR-PEN; Procollagen-proline, 2-oxyglutarate 4-dioxygenase | |
| | (proline 4-hydrolase), beta polypeptide (protein disulfide isomerase) | |
| | -related” | |
| P5 | protein disulfide isomerase-related protein | |
| PACSIN2 | protein kinase C and casein kinase substrate in neurons 2 | |
| PAFAHIB1 | “platelet-activating factor acetylhydrolase, isoform Ib, alpha subunit | |
| | (45 kD); LIS1; PAFAH; lissencephaly 1” | |
| PAFAH1B2 | “platelet-activating factor acetylhydrolase, isoform Ib, beta subunit | |
| | (30 kD)” | |
| PAFAH1B3 | “platelet-activating factor acetylhydrolase, isoform Ib, gamma subunit | |
| | (29 kD)” | |
| PAFAH2 | platelet-activating factor acetyihydrolase 2 (40 kD) | |
| PAH | phenylalanine hydroxylase; Hs.1870 | |
| PAICS | “PAIS; phosphoribosylaminoimidazole carboxylase, | |
| | phosphoribosylaminoribosylaminoimidazole succinocarboxamide | |
| | synthetase” | |
| PAICSP1 | “phosphoribosylaminoimidazole carboxylase, | |
| | phosphoribosylaminoimidazole succinocarboxamide synthetase | |
| | carboxylase pseudogene 1” | |
| PAICSP2 | “phosphoribosylaminoimidazole carboxylase, | |
| | phosphoribosylaminoimidazole succinocarboxamide synthetase | |
| | pseudogene 2” | |
| PAK1 | p21/Cdc42/Rac1-activated kinase 1 (yeast Ste20-related) | |
| PAK2 | p21 (CDKN1A)-activated kinase 2; hPAK65 | |
| PAK3 | “MRX30; p21 (CDKN1A)-activated kinase 3; mental retardation, X- | |
| | linked 30; bPAK; hPAK3” | |
| PAK4 | “protein kinase related to S. cerevisiac STE20, effector for Cdc42Hs” | |
| PAM | Hs.83920; peptidylglycine alpha-amidating monooxygenase | |
| PAP | poly(A) polymerase | |
| PAPSS1 | 3′-phosphoadenosine 5′-phosphosulfate synthase 1; PAPSS; ATPSK1 | |
| PAPSS2 | SK2; ATPSK2; 3-prime-phosphoadenosine 5-prime-phosphosulfate | |
| | synthase 2 | |
| PARG | poly (ADP-ribose) glycohydrolase | |
| PARK2 | “Parkinson disease (autosomal recessive, juvenile) 2; PDJ; AR-JP; parkin” | |
| PARK3 | “Parkinson disease, dominant Lewy-body, 3” | |
| PARN | poly(A)-specific ribonuclease (deadenylation nuclease) | |
| PC | Hs.89890; pyruvate carboxylase; PCB | |
| PC4 | PC4-LSB; activated RNA polymerase II transcription cofactor; activated | |
| | RNA polymerase II transcription cofactor 1; activated RNA polymerase | |
| | II transcription cofactor 4; P15 | |
| PCBD | Hs.3192; PCD; DCOH; 6-pyruvoyl-tetrahydropterin synthase/dimeriza- | |
| | tion cofactor of hepatocyte nuclear factor 1 alpha (TCF1); pterin-4 | |
| | -alpha carbinolamine dehydratase | |
| PCCA | “Hs.80741; propionyl Coenzyme A carboxylase, alpha polypeptide” | |
| PCCB | “Hs.63788; propionyl Coenzyme A carboxylase, beta polypeptide” | |
| PCK1 | Hs.1872; phosphoenolpyruvate carboxykinase 1 (soluble) | |
| PCK2 | PEPCK; phosphoenolpyruvate carboxykinase 2 (mitochondrial) | |
| PCLD | PLD1; polycystic liver disease; PLD | |
| PCMT1 | protein-L-isoaspartate (D-aspartate) O-methyltransferase | |
| PCOLC | procollagen C-endopeptidase | |
| PCOLCE | procollagen C-endopeptidase enhancer; Hs.91299 | |
| PCOLN3 | procollagen (type III) N-endopeptidase | |
| PCSK1 | Hs.78977; PC1; NEC1; PC-1; proprotein convertase subtilisin/kexin type | |
| | 1 | |
| PCSK2 | Hs.93164; PC2; NEC2; PC-2; proprotein convertase subtilisin/kexin type | |
| | 2 | |
| PCSK3 | proprotein convertase subtilisin/kexin type 3 | |
| PCSK4 | PC4; proprotein convertase subtilisin/kexin type 4 | |
| PCSK5 | proprotein convertase subtilisin/kexin type 5 | |
| PCSK7 | PC8; PC7; LPC; SPC7; proprotein convertase subtilisinlkexin type 7; | |
| | Lymphoma Proprotein Convertase | |
| PCTK1 | 1; PCTGAIRE; PCTAIRE protein kinease 1 | |
| PCTK2 | PCTAIRE protein kinease 2 | |
| PCTK3 | Hs.2994; 3; PCTAIRE; protein kinease 3 | |
| PCYT1A | “PCYT1; phosphate cytidylyltransferase 1, choline; CT; CTPCT” | |
| PCYT1B | “CCT-BETA; phosphate cytidylyltransfearse 1, choline; beta isoform” | |
| PCYT2 | “phosphate cytidylyltransferase 2, ethanolamine; ET” | |
| PDB1 | PDB; Paget disease of bone 1 | |
| PDB2 | Paget disease of bone 2 | |
| PDE10A | phosphodiesterase 10A | |
| PDE1A | “phosphodiesterase 1A, calmodulin-dependent; Hs.41717; Human 3′,5′ | |
| | cyclic nucleotide phosphodiesterase (HSPDE1A3A)” | |
| PDE1B | “PDES1B; phosphodiesterase 1B, calmodulin-dependent” | |
| PDE1C | “phosphodiesterase 1C, calmodulin-dependent (70 kD); HCAM3; | |
| | Hs.41718; Human 3′,5′ cyclic nucleotide phosphodiesterase | |
| | (HSPDE1C1A)” | |
| PDE2A | ”phosphodiesterase 2A, cGMP-stimulated; Hs.3831; Human cGMP- | |
| | stimulated 3′,5′-cyclic nucleotide phosphodiesterase PDE2A3 (PDE2A) | |
| | mRNA, complete cds” | |
| PDE3A | “phosphodiesterase 3A, cGMP-inhibited; CGI-PDE” | |
| PDE3B | “phosphodiesterase 3B, cGMP-inhibited” | |
| PDE4A | “Hs.96083; DPDE2; phosphodiesterase 4A, cAMP-specific (dunce | |
| | (Drosophila)-homolog phosphodiesterase E2)” | |
| PDE4B | “Hs.188; DPDE4; PDEIVB; phosphodiesterase 4B, cAMP-specific | |
| | (dunce (Drosophila)-homolog phosphodiesterase E4)” | |
| PDE4C | “Hs.189; DPDE1; phosphodiesterase 4C, cAMP-specific (dunce | |
| | (Drosophila)-homolog phosphodiesterase E1)” | |
| PDE4D | “DPDE3; phosphodiesterase 4D, cAMP-specific (dunce (Drosophila)- | |
| | homolog phosphodiesterase E3)” | |
| PDE5A | “phosphodiesterase SA, cGMP-specific” | |
| PDE6A | “phosphodiesterase 6A, cGMP-specific, rod, alpha; PDEA” | |
| PDE6B | “phosphodiesterase 6B, cGMP-specific, rod, beta (congenital stationary | |
| | night blindness 3, autosomal dominant); Hs.2593; CSNB3; PDEB” | |
| PDE6C | “phosphodiesterase 6C, cGMP-specific, cone, alpha prime” | |
| PDE6D | “phosphodiesterase 6D, cGMP-specific, rod, delta” | |
| PDE6G | “phosphodiesterase 6G, cGMP-specific, rod, gamma; Hs.1857; PDEG” | |
| PDE6H | “phosphodiesterase 6H, cGMP-specific, cone, gamma” | |
| PDE7A | phosphodiesterase 7A; HCP1 | |
| PDE8A | phosphodiesterase 8A | |
| PDE8B | phosphodiesterase 8B | |
| PDE9A | phosphodiesterase 9A | |
| PDHA1 | Hs.1023; PDHA; pyruvate dehydrogenase (lipoamide) alpha 1 | |
| PDHA2 | PDHAL; pyruvate dehydrogenase (lipoamide) alpha 2 | |
| PDHB | Hs.979; pyruvate dehydrogenase (lipoamide) beta | |
| PDI | PDI-PEN; protein disulfide isomerase(pancreas) | |
| PDI2 | “KIAA0994; peptidyl arginine deiminase, type II” | |
| PDIR | for protein disulfide isomerase-related | |
| PDK1 | “pyruvate dehydrogenase kinase, isoenzyme 1; Hs.81233” | |
| PDK2 | “pyruvate dehydrogenase kinase, isoenzyme 2” | |
| PDK3 | “pyruvate dehydrogenase kinase, isoenzyme 3” | |
| PDK4 | “pyruvate dehydrogenase kinase, isoenzyme 4; Hs.57695” | |
| PDNP1 | NPPS; M6S1; PC-1; phosphodiesterase I/nucleotide pyrophosphatase 1 | |
| | (homologous to mouse Ly-41 antigen) | |
| PDNP2 | ATX; phosphodiesterase I/nucleotide pyrophosphatase 2 (autotaxin); | |
| | autotaxin; PD-IALPHA | |
| PDNP3 | phosphodiesterase I/nucleotide pyrophosphatase 3; PD-IBETA | |
| PDPK1 | PDK1; PkB kinase | |
| PDX1 | “pyruvate dehydrogenase complex, component X; protein X” | |
| PDXK | “pyridoxal (pyridoxine, vitamin B6) kinase; PKH; PNK” | |
| PECI | “peroxisomal D3,D2-enoyl-CoA isomerase” | |
| PEMT | phosphatidylethanolamine N-methyltransferase; PEMT2; PEMPT | |
| PEN11B | putative serine/threonine protein kinase | |
| PEPA | peptidase A | |
| PEPB | peptidase B | |
| PEPC | peptidase C | |
| PEPD | Hs.73947; peptidase D | |
| PEPE | peptidase E | |
| PEPS | peptidase S | |
| PFAS | phosphoribosylformylglycinamidine synthase (FGAR amidotransferase); | |
| | A putative Human homolog of | |
| | PHOSPHORIBOSYLFORMYLGLYCINAMIDE SYNTHASE; PURL; | |
| | KIAA0361; FGARAT | |
| PFKFB1 | “Hs.739; PFRX; 6-phosphoftucto-2-kinase/fructose-2,6-biphosphatase 1” | |
| PFKFB2 | “6-phosphofructo-2-kinase/fructose-2,6-biphosphatase 2” | |
| PFKFB3 | “6-phosphofructo-2-kinase/fructose-2,6-biphosphatase 3” | |
| PFKFB4 | “6-phosphofructo-2-kinase/fructose-2,6-biphosphatase 4” | |
| PFKL | “phosphofructokinase, liver; Hs.100005” | |
| PFKM | “Hs.75160; phosphofructokinase, muscle” | |
| PFKP | “Hs.99910; phosphofructokinase, platelet; Hs.75363” | |
| PFKX | “phosphofructokinase polypeptide X” | |
| PFTK1 | PFTAIRE protein kinase 1 | |
| PGAM1 | Hs.74575; PGAMA; phosphoglycerate mutase 1 (brain) | |
| PGAM2 | Hs.46039; phosphoglycerate mutase 2 (muscle) | |
| PGCP | plasma glutamate carboxypeptidase | |
| PGD | Hs.75888; phosphogluconate dehydrogenase | |
| PGDL1 | phosphogluconate dehydrogenase-like 1 | |
| PGGT1B | “protein geranylgeranyltransferase type I, beta subunit; GGTI; BGGI” | |
| PGK1 | Hs.78771; phosphoglycerate kinase 1 | |
| PGK1P1 | “phosphoglycerate kinase 1, pseudogene 1” | |
| PGK1P2 | “phosphoglycerate kinase 1, pseudogene 2” | |
| PGK2 | phosphoglycerate kinase 2 | |
| PGM1 | phosphoglucomutase 1; Hs.1869 | |
| PGM2 | phosphoglucomutase 2 | |
| PGM3 | phosphoglucomutase 3 | |
| PGM5 | phosphoglucomutase 5 | |
| PGP | phosphoglycolate phosphatase | |
| PGS1 | Phosphatidylglycerophosphate Synthase | |
| PHEX | “HYP; phosphate regulating gene with homologies to endopeptidases on | |
| | the X chromosome (hypophosphatemia, vitamin D resistant rickets); | |
| | PEX; HPDR” | |
| PHGDH | phosphoglycerate dehydrogenase; PGAD; 3-phosphoglycerate | |
| | dehydrogenase; PDG; PGDH; SERA | |
| PHKA1 | “phosphorylase kinase, alpha 1 (muscle); Hs.2393; PHKA; | |
| | phosphorylase kinase, alpha 1 (muscle), muscle glycogenosis” | |
| PHKA2 | “PHK; phosphorylase kinase, alpha 2 (liver); phosphorylase kinase | |
| | deficiency, liver (glycogen storage disease type VIII); PYK; XLG; | |
| | XLG2; PYKL; phosphorylase kinase, alpha 2 (liver), glycogen storage | |
| | disease IX” | |
| PHKB | “phosphorylase kinase, beta” | |
| PHKBP1 | “phosphorylase kinase, beta pseudogene 1” | |
| PHKBP2 | “phosphorylase kinase, beta pseudogene 2” | |
| PHKG1 | “PHKG; phosphorylase kinase, gamma 1 (muscle)” | |
| PHKG2 | “Hs.87452; phosphorylase kinase, gamma 2 (testis)” | |
| PHKGL | “phosphorylase kinase, gamma-like” | |
| PHYH | phytanoyl-CoA hydroxylase (Refsum disease); PAHX; Refsum disease | |
| PI | “Hs.102128; PI1; protease inhibitor 1 (anti-elastase), alpha-1-antitrypsin; | |
| | Hs.75621; Hs.99978; Hs.100021” | |
| PI10 | “protease inhibitor 10 (ovalbumin type, bomapin)” | |
| PI12 | protease inhibitor 12 (neuroserpin) | |
| PI13 | protease inhibitor 13 | |
| PI14 | protease inhibitor 14 (pancpin) | |
| PI3 | “Hs.37072; ESI; SKALP; ELAFIN; protease inhibitor 3, skin-derived | |
| | (SKALP)” | |
| PI4 | protease inhibitor 4 (kallistatin) | |
| PI5 | protease inhibitor 5 (maspin); Hs.55279 | |
| PI6 | PTI; CAP; protease inhibitor 6 (placental thrombin inhibitor) | |
| PI7 | PNI; protease inhibitor 7 (protease nexin I) | |
| PI8 | protease inhibitor 8 (ovalbumin type); CAP-2 | |
| PI8L1 | protease inhibitor 8 (ovalbumin type)-like 1 | |
| PI8L2 | protease inhibitor 8 (ovalbumin type)-like 2 | |
| PI9 | CAP-3; protease inhibitor 9 (ovalbumin type) | |
| PICK1 | “Protein that Interacts with C Kinase, 1” | |
| PIG3 | quinone oxidoreductase homolog | |
| PIG6 | proline oxidase homolog | |
| PIK3C2A | “phosphatidylinositol 3-kinase, class 2, alpha polypeptide” | |
| PTK3C2B | “phosphatidylinositol 3-kinase, class 2, beta polypeptide; C2-PI3K; | |
| | PI3K- C2beta” | |
| PIK3C2G | “phosphatidylinositol 3-kinase, class 2, gamma polypeptide” | |
| PIK3C3 | “phosphatidylinositol 3-kinase, class 3; Vps34” | |
| PIK3CA | “phosphatidylinositol 3-kinase, catalytic, alpha polypeptide” | |
| PIK3CB | “phosphatidylinositol 3-kinase, catalytic, beta polypeptide; PIK3C1” | |
| PIK3CD | “phosphatidylinositol 3-kinase, catalytic, delta polypeptide; p110d” | |
| PIK3CG | “phosphatidylinositol 3-kinase, catalytic, gamma polypeptide; Hs.32942” | |
| PIK3R1 | “phosphatidylinositol 3-kinase, regulatory subunit, polypeptide 1 (p85 | |
| | alpha); GRB1” | |
| PIK3R2 | “P85B; phosphatidylinositol 3-kinase, regulatory subunit, polypeptide 2 | |
| | (p85 beta)” | |
| PIK3R3 | “phosphoinositide-3-kinase, regulatory subunit, polypeptide 3 (p55, | |
| | gamma)” | |
| PIK4CA | “phosphatidylinositol 4-kinase, catalytic, alpha polypeptide; PI4K- | |
| | ALPHA” | |
| PIK4CB | “phosphatidylinositol 4-kinase, catalytic, beta polypeptide; PI4Kbeta” | |
| PIL | protease inhibitor 1 (alpha-1-antitrypsin)-like | |
| PIN | associated protein inhibitor of neuronal nitric oxide synthase | |
| PIN1 | “peptidyl-prolyl cis/trans isomerase, NIMA-interacting; dod” | |
| PIN1L | “peptidyl-prolyl cis/trans isomerase, NIMA-interacting-like” | |
| PIN1L2 | protein (peptidyl-prolyl cis/trans isomerase) NIMA-interacting 1-like 2 | |
| PIN4 | “protein (peptidyl-prolyl cis/trans isomerase) NIMA-interacting, 4 | |
| | (parvulin)” | |
| PIP5K1A | “phosphatidylinositol-4-phosphate 5-kinase, type I, alpha” | |
| PIP5K1B | “phosphatidylinositol-4-phosphate 5-kinase, type I, beta; MSS4 protein | |
| | (Saccharomyces cerevisiae) homolog; STM7-LSB; STM7; MSS4” | |
| PIP5K1C | “phosphatidylinositol-4-phosphate 5-kinase, type I, gamma; KIAA0589” | |
| PIP5K2A | “phosphatidylinositol-4-phosphate 5-kinase, type II, alpha” | |
| PIP5K2B | “phosphatidylinositol-4-phosphate 5-kinase, type II, beta; PIP5KIIB” | |
| PIS | phosphatidylinositol synthase | |
| PK428 | ser-Thr protein kinase related to the myotonic dystrophy protein kinase | |
| PKD1 | Hs.75813; PBP; polycystic kidney disease 1 (autosomal dominant) | |
| PKD2 | polycystic kidney disease 2 (autosomal dominant)-Note: redefinition of | |
| | symbol; Hs.82001; PKD4 | |
| PKD2L1 | PKD2L; PKDL; polycystic kidney disease 2-like 1; polycystin-like | |
| PKD3 | polycystic kidney disease 3 (autosomal dominant); APKD3 | |
| PKDREJ | “polycystic kidney disease (polycystin) and REJ (sperm receptor for egg | |
| | jelly, sea urchin homolog)-like" | |
| PKDTS | “polycystic kidney disease, infantile severe, with tuberous sclerosis” | |
| PKHD1 | ARPKD; polycystic kidney and hepatic disease 1 (autosomal recessive) | |
| PKIA | “PRKACN1; protein kinase, cAMP-dependent, catalytic, inhibitor 1” | |
| PKIB | “PRKACN2; protein kinase, cAMP-dependent, catalytic, inhibitor 2” | |
| PKIG | “protein kinase (cAMP-dependent, catalytic) inhibitor gamma” | |
| PKLR | “Hs.95990; pyruvate kinase, liver and RBC” | |
| PKM2 | “pyruvate kinase, muscle; Hs.990; OIP3” | |
| PKMYT1 | MYT1; membrane-associated tyrosine- and threonine-specific cdc2- | |
| | inhibitory kinase | |
| PLA2G10 | “phospholipase A2, group X; GXPLA2” | |
| PLA2G1B | “PLA2; PPLA2; PLA2A; phospholipase A2, group IB (pancreas)” | |
| PLA2G2A | “phospholipase A2, group IIA (platelets, synovial fluid); Hs.76422; | |
| | PLA2L; PLA2B” | |
| PLA2G2C | “phospholipase A2, group IIC (possible pseudogene)” | |
| PLA2G2D | “phospholipase A2, group IID; secretory phospholipase A2s; sPLA2S” | |
| PLA2G4A | “PLA2G4; phospholipase A2, group IVA (cytosolic, calcium-dependent); | |
| | Hs.3278; phospholipase A2, group IV” | |
| PLA2G4B | “CPLA2-BETA; phospholipase A2, group IVB (cytosolic)” | |
| PLA2G4C | “CPLA2-GAMMA; phospholipase A2, group IVC (cytosolic, calcium- | |
| | independent)” | |
| PLA2G5 | “phospholipase A2, group V” | |
| PLA2G6 | “phospholipase A2, group VI; iPLA2” | |
| PLA2G7 | “phospholipase A2, group VII (platelet-activating factor acetylhydrolase, | |
| | plasma); PAFAH; LDL-PLA2; phospholipase A2, group VII (platelet- | |
| | activating factor acetylhydrolase, plasma)” | |
| PLA2R1 | “PLA2R; phospholipase A2 receptor 1, 180 kD” | |
| PLAA | phospholipase A2-activating protein; PLAP; phospholipase A2-activating | |
| | protein | |
| PLAU | “Hs.77274; plasminogen activator, urokinase” | |
| PLAUR | “Hs.89857; plasminogen activator, urokinase receptor; Hs.83170” | |
| PLCB2 | “Hs.994; phospholipase C, beta 2” | |
| PLCB3 | “phospholipase C, beta 3 (phosphatidylinositol-specific)” | |
| PLCB4 | “Hs.74014; phospholipase C, beta 4” | |
| PLCD1 | “phospholipase C, delta 1” | |
| PLCD3 | “phospholipase C, delta 3” | |
| PLCE | “phospholipase C, epsilon; PLC-L” | |
| PLCG1 | “Hs.993; PLC1; phospholipase C, gamma 1 (formerly subtype 148)” | |
| PLCG2 | “phospholipase C, gamma 2 (phosphatidylinositol-specific); Hs.75648” | |
| PLD1 | “phospholipase D1, phosphatidyicholine-specific” | |
| PLD2 | phospholipase D2 | |
| PLK | polo (Drosophia)-like kinase | |
| PLOD | “procollagen-lysine, 2-oxoglutarate 5-dioxygenase (lysine hydroxylase, | |
| | Ehlers-Danlos syndrome type VI); Hs.75093; LLH; LH” | |
| PLOD2 | “procollagen-lysine, 2-oxoglutarate 5-dioxygenase (lysine hydroxylase) 2” | |
| PLOD3 | “procollagen-lysine, 2-oxoglutarate 5-dioxygenase (lysine hydroxylase) | |
| | 3; LH3” | |
| PLP1 | “PLP; proteolipid protein (Pelizaeus-Merzbacher disease, spastic | |
| | paraplegia 2, uncomplicated); Hs.1787; SPG2” | |
| PLSCR1 | phospholipid scramblase 1 | |
| PMM1 | phosphomannomutase 1; Hs.75835 | |
| PMM2 | CDG1; phosphomannomutase 2; CDGS; carbohydrate-deficient | |
| | glycoprotein syndrome 1 | |
| PMM2P1 | phosphomannomutase 2 pseudogene 1; PMM2psi | |
| PMPCB | MPPB; MPP11; MPPP52; peptidase (mitochondrial processing) beta | |
| PMS1 | PMSL1; postmeiotic segregation increased (S. cerevisiae) 1 | |
| PMS2 | PMSL2; postmeiotic segregation increased (S. cerevisiae) 2 | |
| PMS2L1 | postmeiotic segregation increased 2-like 1; PMS3 | |
| PMS2L10 | postmeiotic segregation increased 2-like 10; PMSR4 | |
| PMS2L11 | postmeiotic segregation increased 2-like 11; PMSR6 | |
| PMS2L12 | postmeiotic segregation increased 2-like 12; PMSL12 | |
| PMS2L2 | postmeiotic segregation increased 2-like 2; PMS4 | |
| PMS2L3 | postmeiotic segregation increased 2-like 3; PMS5 | |
| PMS2L4 | postmeiotic segregation increased 2-like 4; PMS6 | |
| PMS2L5 | postmeiotic segregation increased 2-like 5; PMS7 | |
| PMS2L6 | postmeiotic segregation increased 2-like 6; PMS8 | |
| PMS2L7 | postmeiotic segregation increased 2-like 7; PMSR1 | |
| PMS2L8 | postmeiotic segregation increased 2-like 8; PMSR2 | |
| PMS2L9 | postmeiotic segregation increased 2-like 9; PMSR3 | |
| PMS2LP1 | postmeiotic segregation increased 2-like pseudogene 1; PMSR5 | |
| PMS2LP2 | postmeiotic segregation increased 2-like pseudogene 2; PMSR7 | |
| PMVK | PMK; PMK-PEN; phosphomevalonate kinase | |
| PNKP | polynucleotide kinase 3-prime-phosphatase | |
| PNLIP | Hs.99950; pancreatic lipase; Hs.1108 | |
| PNLIPRP1 | PLRP1; pancreatic lipase-related protein 1 | |
| PNLIPRP2 | PLRP2; pancreatic lipase-related protein 2 | |
| PNMT | Hs.1892; PENT; phenylethanolamine N-methyltransferase | |
| PNMTP1 | phenylethanolamine N-methyltransferase pseudogene 1 | |
| PNPO | PYRIDOXINE-5-PRIME-PHOSPHATE OXIDASE | |
| POLA | “Hs.81942; polymerase (DNA directed), alpha” | |
| POLB | “Hs.1894; polymerase (DNA directed), beta” | |
| POLD1 | “Hs.65383; POLD; polymerase (DNA directed), delta 1, catalytic | |
| | subunit (125 kD)” | |
| POLD2 | “polymerase (DNA directed), delta 2, regulatory subunit (50 kD)” | |
| POLE | “polymerase (DNA directed), epsilon” | |
| POLE2 | “polymerase (DNA directed), epsilon 2; DPE2” | |
| POLG | “Hs.80961; polymerase (DNA directed), gamma” | |
| POLG2 | “polymerase (DNA directed), gamma 2, accessory subunit; HP55; | |
| | POLB; MTPOLB; polymerase (DNA directed), gamma 2, accessory | |
| | subunit” | |
| POLH | “polymerase (DNA directed), eta; XP-V; RAD30A” | |
| POLI | RAD3GB; polymerase (DNA directed) iota; RAD30 (S. cerevisiae) | |
| | homolog B | |
| POLQ | “polymerase (DNA-directed), theta” | |
| POLR2A | polymerase (RNA) II (DNA directed) polypeptide A (220 kD); | |
| | Hs.60366; POLR2; POLRA | |
| POLR2B | polymerase (RNA) II (DNA directed) polypeptide B (140 kD) | |
| POLR2C | Hs.79402; polymerase (RNA) II (DNA directed) polypeptide C (33 kD) | |
| POLR2D | polymerase (RNA) II (DNA directed) polypeptide D | |
| POLR2E | polymerase (RNA) II (DNA directed) polypeptide E (25 kD) | |
| POLR2F | polymerase (RNA) II (DNA directed) polypeptide F | |
| POLR2G | polymerase (RNA) II (DNA directed) polypeptide G; RPB7 | |
| POLR2H | polymerase (RNA) II (DNA directed) polypeptide H | |
| POLR2I | polymerase (RNA) II (DNA directed) polypeptide I (14.5 kD) | |
| POLR2J | polymerase (RNA) II (DNA directed) polypeptide J (13.3 kD) | |
| POLR2K | polymerase (RNA) II (DNA directed) polypeptide K (7.0 kD) | |
| POLR2L | polymerase (RNA) II (DNA directed) polypeptide L (7.6 kD) | |
| POLRMT | polymerase (RNA) mitochondrial (DNA directed); h-mtRPOL | |
| POMT1 | protein-O-mannosyltransferase 1 | |
| PON1 | paraoxonase 1; PON | |
| PON2 | paraoxonase 2 | |
| PON3 | paraoxonase 3 | |
| POR | P450 (cytochrome) oxidoreductase | |
| PP | pyrophosphatase (inorganic) | |
| PP2C-DELTA | “protein phosphatase 2c, delta isozym” | |
| PPAP2A | PAP-2A; phosphatidic acid phosphatase type 2a | |
| PPAP2B | PAP-2B; phosphatidic acid phosphatase type 2b | |
| PPAP2C | PAP-2C; phosphatidic acid phosphatase type 2c | |
| PPAT | Hs.311; GPAT; phosphoribosyl pymphosphate amidotransferase | |
| PPATP1 | phosphoribosyl pyrophosphate amidotransferase pseudogene 1 | |
| PPEF1 | “PPEF; protein phosphatase, EF hand calcium-binding domain 1; PPEF-1” | |
| PPEF2 | “protein phosphatase, EF hand calcium-binding domain 2; PPEF-2” | |
| PPFIA1 | “protein tyrosine phosphatase, receptor type, f polypeptide (PTPRF), | |
| | interacting protein (liprin), alpha 1” | |
| PPFIA2 | “protein tyrosine phosphatase, receptor type, f polypeptide (PTPRF), | |
| | interacting protein (liprin), alpha 2” | |
| PPFIA3 | “protein tyrosine phosphatase, receptor type, f polypeptide (PTPRF), | |
| | interacting protein (liprin), alpha 3; KIAA0654” | |
| PPFIA4 | “protein tyrosine phosphatase, receptor type, f polypeptide (PTPRF), | |
| | interacting protein (liprin), alpha 4” | |
| PPGB | Hs.985; GSL; protective protein for beta-galactosidase (galactosialidosis) | |
| PPIA | peptidylprolyl isomerase A (cyclophilin A) | |
| PPIB | Hs.699; CYPB; peptidylprolyl isomerase B (cyclophilin B) | |
| PPIC | peptidylprolyl isomerase C (cyclophilin C); CYPC | |
| PPID | hCyP40; CYP-40; peptidylprolyl isomerase D (cyclophilin D) | |
| PPIE | CYP-33; peptidyiprolyl isomerase E (cyclophilin E) | |
| PPIF | peptidylprolyl isomerase F (cyclophilin F); CYP3; peptidylprolyl | |
| | isomerase F (cyclophilin F) | |
| PPIL1 | peptidylprolyl isomerase (cyclophilin)-like 1 | |
| PPIP1 | peptidylprolyl isomerase (cyclophilin) pseudogene 1 | |
| PPIP10 | peptidylprolyl isomerase (cyclophilin) pseudogene 10; CRP; | |
| | peptidylprolyl isomerase (cyclophilin) pseudogene 10 | |
| PPIP2 | peptidylprolyl isomerase (cyclophilin) pseudogene 2 | |
| PPIP3 | peptidylprolyl isomerase (cyclophilin) pseudogene 3 | |
| PPIP4 | peptidylprolyl isomerase (cyclophilin) pseudogene 4 | |
| PPIP5 | peptidylprolyl isomerase (cyclophilin) pseudogene 5 | |
| PPIP6 | peptidylprolyl isomerase (cyclophilin) pseudogene 6 | |
| PPIP7 | peptidylprolyl isomerase (cyclophilin) pseudogene 7 | |
| PPIP8 | peptidylprolyl isomerase (cyclophilin) pseudogene 8 | |
| PPIP9 | peptidyiprolyl isomerase (cyclophilin) pseudogene 9 | |
| PPM1A | “protein phosphatase 1A (formerly 2C), magnesium-dependent, alpha | |
| | isoform” | |
| PPM1B | “protein phosphatase 1B (formerly 2C), magnesium-dependent, beta | |
| | isoform” | |
| PPM1D | “WIP1; protein phosphatase 1D magnesium-dependent, delta isoform” | |
| PPM1G | “protein phosphatase 1G (formerly 2C), magnesium-dependent, gamma | |
| | isoform; PPP2CG; protein phosphatase 2, catalytic subunit, gamma | |
| | isoform; PP2Cgamma” | |
| PPM2C | “protein phosphatase 2C, magnesium-dependent, catalytic subunit” | |
| PPMT | PCCMT; HSTE14; prenylcysteine carboxyl methlytransferase | |
| PPOX | PPO; protoporphyrinogen oxidase | |
| PPP1CA | “Hs.78092; PPP1A; protein phosphatase 1, catalytic subunit, alpha | |
| | isoform” | |
| PPP1CB | “Hs.21537; protein phosphatase 1, catalytic subunit, beta isoform” | |
| PPP1CC | “Hs.79081; protein phosphatase 1, catalytic subunit, gamma isoform” | |
| PPP1R10 | “protein phosphatase 1, regulatory subunit 10; FB19; PNUTS” | |
| PPP1R1A | “protein phosphatase 1, regulatory (inhibitor) subunit 1A” | |
| PPP1R1B | “DARPP-32; protein phosphatase 1, regulatory (inhibitor) subunit 1B | |
| | (dopamine and cAMP regulated phosphoprotein, DARPP-32)” | |
| PPP1R2 | “protein phosphatase 1, regulatory (inhibitor) subunit 2” | |
| PPP1R2P | “IPP-2P; protein phosphatase 1, regulatory (inhibitor) subunit 2 | |
| | pseudogene” | |
| PPP1R3 | “protein phosphatase 1, regulatory (inhibitor) subunit 3 (glycogen and | |
| | sarcoplasmic reticulum binding subunit, skeletal muscle); Hs.54496; | |
| | PPP1R3A” | |
| PPP1R5 | “protein phosphatase 1, regulatory (inhibitor) subunit 5” | |
| PPP1R6 | “protein phosphatase 1, regulatory subunit 6 (NOTE: redefinition of | |
| | symbol)” | |
| PPP1R7 | “protein phosphatase 1, regulatory subunit 7; sds22” | |
| PPP1R8 | “protein phosphatase 1, regulatory (inhibitor) subunit 8; ARD1; ard-1; | |
| | NIPP-1” | |
| PPP1R8P | “protein phosphatase 1, regulatory (inhibitor) subunit 8 pseudogene” | |
| PPP1R9 | “protein phosphatase 1, regulatory subunit 9, spinophilin” | |
| PPP2CA | “Hs.78852; protein phosphatase 2 (formerly 2A), catalytic subunit, alpha | |
| | isoform” | |
| PPP2CB | “protein phosphatase 2 (formerly 2A), catalytic subunit, beta isoform” | |
| PPP2CBP | “protein phosphatase 2 (formerly 2A), catalytic subunit, beta isoform | |
| | pseudogene” | |
| PPP2R1A | “protein phosphatase 2 (formerly 2A), regulatory subunit A (PR 65), | |
| | alpha isoform" | |
| PPP2R1B | “protein phosphatase 2 (formerly 2A), regulatory subunit A (PR 65), | |
| | beta isoform; Hs.89608” | |
| PPP2R2A | “Hs.75200; protein phosphatase 2 (formerly 2A), regulatory subunit | |
| | B (PR52), alpha isoform” | |
| PPP2R2B | “protein phosphatase 2 (formerly 2A), regulatory subunit B (PR 52), | |
| | beta isoform” | |
| PPP2R2C | “protein phosphatase 2 (formerly 2A), regulatory subunit B (PR 52), | |
| | gamma isoform” | |
| PPP2R3 | “Hs.89; protein phosphatase 2 (formerly 2A), regulatory subunit B″ (PR | |
| | 72), alpha isoform and (PR 130), beta isoform” | |
| PPP2R4 | “KIAA0044; Hs.78978; PTPA; protein phosphatase 2A, regulatory | |
| | subunit B′ (PR 53)” | |
| PPP2R5A | “protein phosphatase 2, regulatory subunit B (B56), alpha isoform” | |
| PPP2R5B | “protein phosphatase 2, regulatory subunit B (B56), beta isoform” | |
| PPP2R5C | “protein phosphatase 2, regulatory subunit B (B56), gamma isoform” | |
| PPP2R5D | “protein phosphatase 2, regulatory subunit B (B56), delta isoform” | |
| PPP2R5E | “protein phosphatase 2, regulatory subunit B (B56), epsilon isoform” | |
| PPP3CA | “Hs.92; CALN; CNA1; CCN1; CALNA; PPP2B; protein phosphatase 3 | |
| | (formerly 2B), catalytic subunit, alpha isoform (calcineurin A alpha)” | |
| PPP3CB | “protein phosphatase 3 (formerly 2B), catalytic subunit, beta isoform | |
| | (calcineurin A beta); Hs.1335; CALNB” | |
| PPP3CC | “protein phosphatase 3 (formerly 2B), catalytic subunit, gamma isoform | |
| | (calcineurin A gamma)” | |
| PPP3R1 | “protein phosphatase 3 (formerly 2B), regulatory subunit B (19 kD), | |
| | alpha isoform (calcineurin B, type I” | |
| PPP3R2 | “protein phosphatase 3 (formerly 2B), regulatory subunit B (19 kD), | |
| | beta isoform (calcineurin B, type II)” | |
| PPP4C | “PP4; Hs.2903; protein phosphatase 4 (formerly X), catalytic subunit” | |
| PPP4R1 | “protein phosphatase 4, regulatory subunit 1; PP4R1” | |
| PPP5C | “protein phosphatase 5, catalytic subunit; Hs.75180; PPP5” | |
| PPP6C | “protein phosphatase 6, catalytic subunit” | |
| PPT | “palmitoyl-protein thioesterase (ceroid-lipofuscinosis, neuronal 1, | |
| | infantile; Haltia-Santavuori disease); CLN1; INCL” | |
| PPT2 | palmitoyl-protein thioesterase 2 | |
| PRCP | prolylcarboxypeptidase (angiotensinase C); PCP; HUMPCP | |
| PREP | prolyl endopeptidase; Hs.86978; PEP | |
| PRIM1 | primase polypeptide 1 (49 kD); Hs.82741 | |
| PRIM1P1 | “primase polypeptide 1, pseudogene 1” | |
| PRIM2A | Hs.74519; PRIM2; primase polypeptide 2A (58 kD) | |
| PRIM2B | PRIM2; primase polypeptide 2B (58 kD) | |
| PRKA1 | protein kinase A1 | |
| PRKA2 | protein kinase A2 | |
| PRKAA1 | “protein kinase, AMP-activated, alpha 1 catalytic subunit; AMPK alpha 1” | |
| PRKAA2 | “PRKAA; protein kinase, AMP-activated, alpha 2 catalytic subunit; | |
| | protein kinase, AMP-activated; AMPK” | |
| PRKAB1 | “protein kinase, AMP-activated, beta 1 non-catalytic subunit; AMPK | |
| | beta 1” | |
| PRKAB2 | “protein kinase, AMP-activated, beta 2 non-catalytic subunit; AMPK | |
| | beta 2” | |
| PRKACA | “Hs.77271; protein kinase, cAMP-dependent, catalytic, alpha” | |
| PRKACB | “Hs.1903; protein kinase, cAMP-dependent, catalytic, beta” | |
| PRKACG | “protein kinase, cAMP-dependent, catalytic, gamma” | |
| PRKAG1 | “protein kinase, AMP-activated, gamma 1 non-catalytic subunit; AMPK | |
| | gamma 1” | |
| PRKAG2 | “protein kinase, AMP-activated, gamma 2 non-catalytic subunit; AMPK | |
| | gamma 2” | |
| PRKAR1A | “Hs.62039; TSE1; PRKAR1; protein kinase, cAMP-dependent, | |
| | regulatory, type I, alpha (tissue specific extinguisher 1); CNC1; Carney | |
| | myxoma-endocrine complex, type 1” | |
| PRKAR1AP | “protein kinase, cAMP-dependent, regulatory, type I, alpha pseudogene” | |
| PRKAR1B | “Hs.1519; protein kinase, cAMP-dependent, regulatory, type I, beta” | |
| PRKAR2A | “PRKAR2; protein kinase, cAMP-dependent, regulatory, type II, alpha” | |
| PRKAR2B | “Hs.77439; PRKAR2; protein kinase, cAMP-dependent, regulatory, type | |
| | II, beta” | |
| PRKCA | “protein kinase C, alpha; Hs.60762; PKCA” | |
| PRKCB1 | “Hs.77767; PKCB; PRKCB; PRKCB2; protein kinase C, beta 1” | |
| PRKCBP1 | protein kinase C binding protein 1; RACK7 | |
| PRKCBP2 | protein kinase C binding protein 2; RACK17 | |
| PRKCD | “Hs.458; protein kinase C, delta” | |
| PRKCDBP | “SRBC; C-RAF-1; protein kinase C, delta binding protein” | |
| PRKCE | “protein kinase C, epsilon” | |
| PRKCG | “protein kinase C, gamma; Hs.2890; PKCG” | |
| PRKCH | “PRKCL; PKC-L; protein kinase C, eta” | |
| PRKCI | “DXS1179E; Hs.1904; PKCI; protein kinase C, iota” | |
| PRKCL1 | protein kinase C-like 1; DBK; PRK1; PKN; serine-threonine kinase N | |
| PRKCL2 | protein kinase C-like 2; PRK2 | |
| PRKCM | “Hs.2891; PKCM; protein kinase C, mu” | |
| PRKCN | “protein kinase C, nu; EPK2; serine-threonine protein kinase; PKCnu” | |
| PRKCQ | “Hs.89615; protein kinase C, theta” | |
| PRKCSH | protein kinase C substrate 80K-H; Hs.1432; G19P1 | |
| PRKCZ | “Hs.78793; protein kinase C, zeta” | |
| PRKDC | “HYRC1; protein kinase, DNA-activated, catalytic polypeptide; XRCC7; | |
| | hyper-radiosensitivity of murine scid mutation, complementing 1; | |
| | DNAPK” | |
| PRKG1 | “PRKG1B; PRKGR1B; protein kinase, cGMP-dependent, type I; PGK; | |
| | cGKI; protein kinase, cGMP-dependent, regulatory, type I, beta” | |
| PRKG2 | “Protein kinase, cGMP-dependent, type II; cGKII; PRKGR2” | |
| PRKR | “Hs.73821; PKR; protein kinase, interferon-inducible double stranded | |
| | RNA dependent” | |
| PRKRA | “protein kinase, interferon-inducible double stranded RNA dependent | |
| | activator; RAX; PACT” | |
| PRKRI | “protein-kinase, interferon-inducible double stranded RNA dependent | |
| | inhibitor; P58” | |
| PRKRIR | “protein-kinase, interferon-inducible double stranded RNA dependent | |
| | inhibitor, repressor of (P58 repressor)” | |
| PRKX | “protein kinase, X-linked; PKX1” | |
| PRKXP1 | “protein kinase, X-linked, pseudogene 1” | |
| PRKXP2 | “protein kinase, X-linked, pseudogene 2” | |
| PRKY | “protein kinase, Y-linked” | |
| PRMT3 | protein arginine N-methyltransferase 3 (hnRNP methyltransferase S. | |
| | cerevisiae)-like 3 | |
| PRNP | “Hs.74621; CJD; PRIP; prion protein (p27-30) (Creutzfeld-Jakob dis- | |
| | ease, Gerstmann-Strausler-Scheinker syndrome, fatal familial insomnia)” | |
| PRODH | proline dehydrogenase (proline oxidase) | |
| PROSC | proline synthetase co-transcribed (bacterial homolog) | |
| PRP4 | PR4H; serine/threonine-protein kinase PRP4 homolog | |
| PRPS1 | Hs.74093; phosphoribosyl pyrophosphate synthetase 1; PRS I; Hs.56 | |
| PRPS1L1 | PRPSL; phosphoribosyl pyrophosphate synthetase 1-like 1 | |
| PRPS1L2 | phosphoribosyl pyrophosphate synthetase 1-like 2 | |
| PRPS2 | Hs.2910; phosphoribosyl pyrophosphate synthetase 2; PRS II | |
| PRPSAP1 | PAP39; phosphoribosyl pyrophosphate synthetase-associated protein 1 | |
| PRPSAP2 | phosphoribosyl pyrophosphate synthetase-associated protein 2; PAP41 | |
| PRSC1 | “protease, cysteine, 1 (legumain); legumain” | |
| PRSM1 | “protease, metallo, 1, 33 kD; KIAA0047; Hs.57302” | |
| PRSM2 | “protease, metallo, 2” | |
| PRSS# | thymus specific serine peptidase | |
| PRSS1 | “Hs.73981; TRY1; cationic trypsinogen; hereditary pancreatitis; protease, | |
| | serine, 1 (trypsin 1); HPC; PCTT” | |
| PRSS11 | “protease, serine, 11 (IGF binding)” | |
| PRSS12 | “BSSP-3; protease, serine, 12 (neurotrypsin, motopsin)” | |
| PRSS15 | “LONP; HLON; LONES; PRSS15-PENDING; protease, serine, 15” | |
| PRSS17 | “PSTS; KLK4; EMSP1; protease, serine, 17 (enamel matrix, prostate)” | |
| PRSS19 | “HNP; protease, serine, 19 (neuropsin/ovasin)” | |
| PRSS2 | “Hs.105977; TRY2; protease, serine, 2 (trypsin 2)” | |
| PRSS21 | “protease, serine, 21 (testisin); TEST1; testisin; ESP-1; serine protease | |
| | from eosinophils” | |
| PRSS3 | “Hs.58247; TRY3; protease, serine, 3 (trypsin 3)” | |
| PRSS4 | “TRY4; protease, serine, 4 (trypsin 4, brain)” | |
| PRSS7 | “protease, serine, 7 (enterokinase); Hs.3113” | |
| PRSS8 | “protease, serine, 8 (prostasin)” | |
| PRSSL1 | “protease, serine-like, 1; NES1” | |
| PRTN3 | “Hs.928; PR-3; ACPA; C-ANCA; proteinase 3 (serine proteinase, | |
| | neutrophil, Wegener granulomatosis autoantigen)” | |
| PSA | puromycin-sensitive aminopeptidase | |
| PSAP | Hs.78575; SAP1; GLBA; prosaposin (variant Gaudier disease and | |
| | variant metachromatic leukodystrophy) | |
| PSEN1 | AD3; presenilin 1 (Alzheimer disease 3); Hs.46464; FAD; S182; PS1 | |
| PSEN2 | AD4; presenilin 2 (Alzheimer disease 4); AD3L; Hs.25363; STM2; PS2; | |
| | Alzheimer's disease 3-like | |
| PSKH1 | putative seine kinase H1 (symbol provisional) | |
| PSMB8 | “proteasome (prosome, macropain) subunit, beta type, 8 (large | |
| | multifunctional protease 7); D6S216; D6S216E; LMP7; RING10” | |
| PSMB9 | “proteasome (prosome, macropain) subunit, beta type, 9 (large | |
| | multifunctional protease 2); LMP2; RING12” | |
| PSMC1 | “proteasome (prosome, macropain) 26S subunit, ATPase, 1; S4; P56” | |
| PSMC2 | “proteasome (prosome, macropain) 26S subunit, ATPase, 2; S7; MSS1” | |
| PSMC3 | “proteasome (prosome, macropain) 26S subunit, ATPase, 3; TBP1” | |
| PSMC3P | “proteasome (prosome, macropain) 26S subunit, ATPase, 3 pseudogene” | |
| PSMC4 | “proteasome (prosome, macropain) 26S subunit, ATPase, 4; S6; TBP7” | |
| PSMC5 | “proteasome (prosome, macropain) 26S subunit, ATPase, 5; S8; P45; | |
| | TRIP1” | |
| PSMC6 | “proteasome (prosome, macropain) 26S subunit, ATPase, 6; p42” | |
| PSMD1 | “proteasome (prosome, macropain) 26S subunit, non-ATPase, 1; S1; | |
| | P112” | |
| PSMD10 | “proteasome (prosome, macropain) 26S subunit, non-ATPase, 10” | |
| PSMD11 | “proteasome (prosome, macropain) 26S subunit, non-ATPase, 11” | |
| PSMD12 | “proteasome (prosome, macropain) 26S subunit, non-ATPase, 12” | |
| PSMD13 | “proteasome (prosome, macropain) 26S subunit, non-ATPase, 13” | |
| PSMD2 | “S2; P97; TRAP2; proteasome (prosome, macropain) 26S subunit, non- | |
| | ATPase, 2” | |
| PSMD3 | “S3; P58; proteasome (jwosome, macropain) 26S subunit, non-ATPase, 3” | |
| PSMD4 | “proteasome (prosome, macropain) 26S subunit, non-ATPase, 4; S5A” | |
| PSMD5 | “S5B; proteasome (prosome, macropain) 26S subunit, non-ATPase, 5” | |
| PSMD6 | “proteasome ( rosome, macropain) 26S subunit, non-ATPase, 6; S10” | |
| PSMD7 | “proteasome (prosome, macropain) 26S subunit, non-ATPase, 7 (Mov34 | |
| | homolog); S12; P40; MOV34” | |
| PSMD8 | “proteasome (prosome, macropain) 26S subunit, non-ATPase, 8; S14” | |
| PSMD9 | “proteasome (prosome, macropain) 26S subunit, non-ATPase, 9” | |
| PSPH | PSP; phosphoserine phosphatase | |
| PSPHL | CO9; phosphoserine phosphatase-like | |
| PSTPIP1 | proline-serine-threonine phosphatase interacting protein 1; CD2 | |
| | cytoplasmic tail-binding protein; H-PIP; PSTPIP; CD2BP1; CD2BP1L; | |
| | CD2BP1S | |
| PSTPIP2 | MAYP; proline-serine-threonine phosphatase interacting protein 2 | |
| PTE1 | peroxisomal acyl-CoA thioesterase; hTE; hNAACTE; thioesterase II | |
| PTEN | phosphatase and tensin homolog (mutated in multiple advanced cancers | |
| | 1); MMAC1 | |
| PTENP1 | “phosphatase and tensin homolog (mutated in multiple advanced cancers | |
| | 1), pseudogene 1; PTH2; PTEN2; psiPTEN; PTEN-rs” | |
| PTER | RPR-1; phosphotriesterase-related | |
| PTGDS | “prostaglandin D2 synthase (21 kD, brain)” | |
| PTGIS | prostaglandin 12 (prostacyclin) synthase; PGIS; CYP8; CYP8A1 | |
| PTGS1 | Hs.88474; prostaglandin-endoperoxide synthase 1 (prostaglandin G/H | |
| | synthase and cyclooxygenase) | |
| PTGS2 | Hs.89581; COX-2; prostaglandin-endoperoxide synthase 2 (prostaglandin | |
| | G/H synthase and cyclooxygenase); COX2; Hs.89495 | |
| PTK2 | FAK; PTK2 protein tyrosine kinase | |
| PTK2B | protein tyrosine kinase 2 beta; PTK; FAK2; CAKB; PYK2; RAFTK | |
| PTK3B | PTK3B protein tyrosine kinase 3B | |
| PTK4 | PTK4 protein tyrosine kinase | |
| PTK5 | PTK5 protein tyrosine kinase | |
| PTK6 | PTK6 protein tyrosine kinase 6; BRK; breast tumor kinase (non-receptor | |
| | protein tyrosine kinase expressed in breast) | |
| PTK7 | Hs.9374; PTK7 protein tyrosine kinase | |
| PTK8 | PTK8 protein tyrosine kinase | |
| PTK9 | protein tyrosine kinase 9; A6 | |
| PTK9L | A6RP; protein tyrosine kinase 9-like (A6-related protein) | |
| PTP-J | PCP-2; PTP-PI; protein tyrosine phosphatase J | |
| PTP4A1 | “protein tyrosine phosphatase type IVA, member 1; PRL-1; PTPCAAX1” | |
| PTP4A2 | “PTP4A; protein tyrosine phosphatase type IVA, member 2; OV-1; PRL- | |
| | 2; RU-PP-1; PTPCAAX2; ptp-IV1a” | |
| PTP4A3 | “PRL-3; protein tyrosine phosphatase type IVA, member 3” | |
| PTP4AP1 | protein tyrosine phosphatase IVA pseudogene 1 | |
| PTP4AP2 | protein tyrosine phosphatase type IVA pseudogene 2 | |
| PTPGMC1 | “protein-tyrosine phosphatase, receptor-type, expressed by glomerular | |
| | mesangial cells” | |
| PTPLA | “protein tyrosine phosphatase-like (proline instead of catalytic arginine), | |
| | member a” | |
| PTPLB | “protein tyrosine phosphatase-like (proline instead of catalytic arginine), | |
| | member b” | |
| PTPLC | “protein tyrosine phosphatase-like (proline instead of catalytic arginine), | |
| | member c” | |
| PTPN1 | “Hs.81444; PTP1B; PTP-1B; protein tyrosine phosphatase, non-receptor | |
| | type 1” | |
| PTPN11 | “Hs.22868; BPTP3; SH-PTP2; protein tyrosine phosphatase, | |
| | non-receptor type 11” | |
| PTPN12 | “protein tyrosine phosphatase, non-receptor type 12; Hs.62; PTPG1; | |
| | PTP-PEST” | |
| PTPN13 | “protein tyrosine phosphatase, nonreceptor type 13; PTP1E; PTP-BAS; | |
| | protein tyrosine phosphatase, non-receptor type 13 (APO-1/CD95 (Fas)- | |
| | associated phosphatase); PTPL1” | |
| PTPN14 | “PEZ; protein tyrosine phosphatase, non-receptor type 14” | |
| PTPN17 | “protein tyrosine phosphatase, non-receptor type 17” | |
| PTPN2 | “Hs.82829; PTPT; TCELLPTP; protein tyrosine phosphatase, non- | |
| | receptor type 2” | |
| PTPN21 | “PTPD1; PTPRL10; protein tyrosine phosphatase, non-receptor type 21” | |
| PTPN2P1 | “PTPTP1; PTPN2P; protein tyrosine phosphatase, non-receptor type 2 | |
| | (pseudogene 1)” | |
| PTPN2P2 | “protein tyrosine phosphatase, non-receptor type 2 (pseudogene 2)” | |
| PTPN3 | “Hs.644; PTPH1; protein tyrosine phosphatase, non-receptor type 3” | |
| PTPN4 | “PTPMEG; protein tyrosine phosphatase, non-receptor type 4 | |
| | (megakaryocyte)” | |
| PTPN5 | “STEP; PTPSTEP; protein tyrosine phosphatase, non-receptor type 5 | |
| | (striatum-enriched)” | |
| PTPN6 | “Hs.63489; HCP; HCPH; PTP-1C; protein tyrosine phosphatase, non- | |
| | receptor type 6 ” | |
| PTPN7 | “Hs.73922; HEPTP; LC-PTP; protein tyrosine phosphatase, non-receptor | |
| | type 7; Hs.35” | |
| PTPN8 | “protein tyrosine phosphatase, non-receptor type 8” | |
| PTPN9 | “Hs.78598; MEG2; protein tyrosine phosphatase, non-receptor type 9” | |
| PTPNS1 | “SIRP; SHPS1; MYD-1; STRP-ALPHA-1; protein tyrosine phosphatase, | |
| | non-receptor type substrate 1” | |
| PTPRA | “Hs.26045; LRP; PTPA; HLPR; HPTPA; RPTPA; PTPRL2; protein | |
| | tyrosine phosphatase, receptor type, alpha polypeptide” | |
| PTPRB | “Hs.10623; PTPB; HPTPB; protein tyrosine phosphatase, receptor type, | |
| | beta polypeptide” | |
| PTPRC | “Hs.62399; LCA; CD45; T200; GP180; protein tyrosine phosphatase, | |
| | receptor type, c polypeptide” | |
| PTPRCAP | “protein tyrosine phosphatase, receptor type, c polypeptide-associated | |
| | protein; LPAP; lymphocyte phosphatase-associated phosphoprotein” | |
| PTPRD | “Hs.15320; HPTPD; protein tyrosine phosphatase, receptor type, delta | |
| | polypeptide” | |
| PTPRE | “HPTPE; protein tyrosine phosphatase, receptor type, epsilon polypeptide” | |
| PTPRF | “Hs.75216; LAR; protein tyrosine phosphatase, receptor type, f | |
| | polypeptide” | |
| PTPRG | “D3S1249; Hs.89627; PTPG; HPTPG; RPTPG; protein tyrosine | |
| | phosphatase, receptor type, gamma polypeptide” | |
| PTPRH | “Hs.328; SAP-1; protein tyrosine phosphatase, receptor type, H” | |
| PTPRJ | “protein tyrosine phosphatase, receptor type, J; DEP1; HPTPeta” | |
| PTPRK | “protein tyrosine phosphatase, receptor type, K; R-PTP-kappa ” | |
| PTPRM | “Hs.7619; RPTPU; PTPRL1; protein tyrosine phosphatase, receptor | |
| | type, mu polypeptide” | |
| PTPRN | “IA-2; protein tyrosine phosphatase, receptor type, N” | |
| PTPRN2 | “protein tyrosine phosphatase, receptor type, N polypeptide 2; | |
| | KIAA0387; A tyrosine phosphatase, phogrin/ICAAR (cf. | |
| | Y08569/JC5062); IAR; ICAAR; PTPRP; phogrin; IA-2beta” | |
| PTPRO | “protein tyrosine phosphatase, receptor type, O; PTPU2; GLEPP1; PTP- | |
| | U2” | |
| PTPRQ | “protein tyrosine phosphatase, receptor type, Q (NOTE: redefinition of | |
| | symbol)” | |
| PTPRR | “PTPRQ; protein tyrosine phosphatase, receptor type, R; protein tyrosine | |
| | phosphatase, receptor type, Q; PTPBR7; PCPTP1; PTP-SL” | |
| PTPRS | “protein tyrosine phosphatase, receptor type, sigma” | |
| PTPRZ1 | “PTPRZ; protein tyrosine phosphatase, receptor-type, zeta polypeptide 1; | |
| | Hs.78867; PTPZ; HPTPZ; PTP18; RPTPB” | |
| PTPRZ2 | “protein tyrosine phosphatase, receptor-type, zeta polypeptide 2 ” | |
| PTRF | polymerase I and transcript release factor | |
| PTS | Hs.366; 6-pyruvoyltetrahydropterin synthase | |
| PTSP1 | PTSP1-PEN; 6-pyruvoyltetrahydropterin synthase pseudogene | |
| PYCR1 | Hs.79217; P5C; PYCR; pyrroline-5-carboxylate reductase 1 | |
| PYCS | pyrroline-5-carboxylate synthetase (glutamate gamma-semialdehyde | |
| | synthetase); Hs.13048; P5CS; GSAS | |
| PYGB | “Hs.75658; phosphorylase, glycogen; brain” | |
| PYGBL | “phosphorylase, glycogen; brain-like” | |
| PYGL | “phosphorylase, glycogen; liver (Hers disease, glycogen storage disease | |
| | type VI); Hs.771” | |
| PYGM | “phosphorylase, glycogen; muscle (McArdle syndrome, glycogen storage | |
| | disease type V)” | |
| PheHB | phenylalanyl-tRNA synthetase beta-subunit | |
| QARS | glutamine-tRNA synthetase | |
| QDPR | quinoid dihydropteridine reductase; Hs.75438; DHPR | |
| QPRT | guinolinate phosphoribosyltransferase | |
| RAB18 | RAB18 small GTPase | |
| RABGGTA | “Rab geranylgeranyl transferase, alpha subunit” | |
| RABGGTB | “Rab geranylgeranyl transferase, beta subunit” | |
| RACGAP1 | MGCRACGAP; Rac GTPase activating protein 1 | |
| RAD53 | CHK2; CDS1; HUCDS1; protein kinase Chk2; checkpoint kinase 2 | |
| RALDH2 | retinaldehyde dehydrogenase 2 | |
| RANGAP1 | Ran GTPase activating protein 1; Fug1 | |
| RAP1GA1 | “Hs.75151; KREV-1; SMGP21; RAP1, GTPase activating protein 1” | |
| RARS | arginyl-tRNA synthetase; Hs.74514 | |
| RASA1 | RASA; Hs.758; RAS p21 protein activator (GTPase activating protein); | |
| | GAP | |
| RASA3 | “GAPIII; RAS p21 protein activator (GTPase activating protein) 3 | |
| | (Ins(1,3,4,5)P4-binding protein)” | |
| RCE1 | FACE-2; prenyl protein protease RCE1 | |
| RDH5 | RDH1; retinol dehydrogenase 5 (11-cis and 9-cis); Hs.33730 | |
| RDHL] | retinol dehydrogenase homolog; RDHL | |
| RDPA | Refsum disease with increased pipecolicacidemia | |
| RECQL | Hs.1536; RecQ protein-like (DNA helicase Q1-like) | |
| RET | “ret proto-oncogene (multiple endocrine neoplasia MEN2A, MEN2B | |
| | and medullary thyroid carcinoma 1, Hirschsprung disease); Hs.6253; | |
| | PTC; MTC1; MEN2A; HSCR1; MEN2B” | |
| REV3L | “REV3 (yeast homolog)-like, catalytic subunit of DNA polymerase zeta; | |
| | POLZ” | |
| RHOK | Hs.103501; GRK1; rhodopsin kinase | |
| RIPK1 | receptor (TNFRSF)-interacting serine-threonine kinase 1; RIP; receptor | |
| | (TNFRSF)-interacting serine-threonine kinase 1 | |
| RIPK2 | RICK; RIP2; CARDIAK; receptor-interacting serine-threonine kinase 2 | |
| RMD1 | rippling muscle disease 1 | |
| RMRP | RNA component of mitochondrial RNA processing endoribonuclease | |
| RNAC | RNA cyclase homolog | |
| RNAH | RNA helicase family | |
| RNAHP | RNA helicase-related protein | |
| RNASE1 | “RNS1; ribonuclease, RNase A family, 1 (pancreatic); Hs.78224” | |
| RNASE2 | “RNS2; ribonuclease, RNase A family, 2 (liver, eosinophil-derived | |
| | neurotoxin); EDN; Hs.728” | |
| RNASE3 | “RNS3; ribonuclease, RNase A family, 3 (eosinophil cationic protein); | |
| | ECP; Hs.73839” | |
| RNASE4 | “ribonuclease, RNase A family, 4” | |
| RNASE6 | “RNS6; ribonuclease, RNase A family, k6” | |
| RNASE6PL | ribonuclease 6 precursor | |
| RNASEH1 | ribonuclease H1; RNH1 | |
| RNASEHI | “ribonuclease H1, large subunit” | |
| RNASEL | “RNS4; ribonuclease L (2′,5′-oligoisoadenylate synthetase-dependent); | |
| | Hs.10716; ribonuclease 4” | |
| RNGTT | RNA guanylyltransferase and 5′-phosphatase; HCE; HCE1; hCAP | |
| RNH | ribonuclease/angiogenin inhibitor; Hs.75108; RAI | |
| RNMT | RNA (guanine-7-) methyltransferase | |
| RNPEP | arginyl aminopeptidase (aminopeptidase B) | |
| ROCK1 | “Rho-associated, coiled-coil containing protein kinase 1; p160ROCK” | |
| ROCK2 | “KIAA0619; Rho-associated, coiled-coil containing protein kinase 2” | |
| RODH | oxidative 3 alpha hydroxysteroid dehydrogenase; retinol dehydrogenase | |
| RODH-4 | microsomal NAD+-dependent retinol dehydrogenase 4 | |
| ROK1 | ATP-dependent RNA helicase | |
| RPA40 | RPA39; RNA polymerase I subunit | |
| RPC | RNA 3′-terminal phosphate cyclase | |
| RPC155 | polymerase (RNA) III (DNA directed) (155 kD) | |
| RPC32 | polymerase (RNA) III (DNA directed) (32 kD) | |
| RPC39 | polymerase (RNA) III (DNA directed) (39 kD) | |
| RPC62 | polymerase (RNA) III (DNA directed) (62 kD) | |
| RPE | ribulose-5-phosphate-3-epimerase | |
| RPGR | RP3; CRD; retinitis pigmentosa 3 (X-linked recessive); Retinitis | |
| | pigmentosa GTPase regulator | |
| RPIA | RPI; ribose 5-phosphate isomerase A (ribose 5-phosphate epimerase) | |
| RPL17L1 | “ribosomal protein L17-like 1, G1-phase expressed” | |
| RPL7AL1 | “ribosomal protein L7A-like 1, G1-phase expressed” | |
| RPP14 | ribonuclease P (14 kD) | |
| RPP30 | ribonuclease P (30 kD) | |
| RPP38 | ribonuclease P (38 kD) | |
| RPP40 | “ribonuclease P, 40 kD subunit” | |
| RPS17L3 | “ribosomal protein S17-like 3, G1-phase expressed” | |
| RPS3L1 | “ribosomal protein S3-like 1, G1-phase expressed” | |
| RPS6KA1 | “ribosomal protein S6 kinase, 90 kD, polypeptide 1; RSK; HU-1; RSK1” | |
| RPS6KA2 | “ribosomal protein S6 kinase, 90 kD, polypeptide 2; Hs.2079; RSK; | |
| | HU-2; RSK3” | |
| RPS6KA3 | “ribosomal protein S6 kinase, 90 kD, polypeptide 3; RSK; HU-2; RSK2; | |
| | HU-3” | |
| RPS6KA4 | “ribosomal protein S6 kinase, 90 kD, polypeptide 4; MSK2; RSK-B; | |
| | ribosomal protein S6 kinase, 90 kD, polypeptide 4” | |
| RPS6KA5 | “ribosomal protein S6 kinase, 90 kD, polypeptide 5; MSK1; RLPK; | |
| | MSPK1; ribosomal protein S6 kinase, 90 kD, polypeptide 5 ” | |
| RPS6KB1 | “ribosomal protein S6 kinase, 70 kD, polypeptide 1” | |
| RPS6KB2 | “ribosomal protein S6 kinase, 70 kD, polypeptide 2” | |
| RPS6KB3 | “ribosomal protein 56 kinase, 70 kD, polypeptide 3” | |
| RRM1 | ribonucleotide reductase Ml polypeptide | |
| RRM2 | Hs.75319; ribonucleotide reductase M2 polypeptide | |
| RRM2P1 | ribonucleotide reductase M2 polypeptide pseudogene 1 | |
| RRM2P2 | ribonucleotide reductase M2 polypeptide pseudogene 2 | |
| RRM2P3 | ribonucleotide reductase M2 polypeptide pseudogene 3 | |
| RRM2P4 | ribonucleotide reductase M2 polypeptide pseudogene 4 | |
| RRP4 | “homolog of Yeast RRP4 (ribosomal RNA processing 4), 3′-5′- | |
| | exoribonuclease” | |
| RYK | D3S3195; Hs.79350; RYK receptor-like tyrosine kinase | |
| RYKL1 | RYK receptor-like tyrosine kinase-like 1 | |
| RYR1 | MHS1; ryanodine receptor 1 (skeletal); RYR; MHS; malignant | |
| | hyperthermia susceptibility 1; sarcoplasmic reticulum calcium release | |
| | gene | |
| S1P | “site-1 protease (subtilisin-like, sterol-regulated, cleaves sterol regulatory | |
| | element binding proteins)” | |
| SARDH | DMGDHL1; sarcosine dehydrogenase; dimethylglycine dehydrogenase- | |
| | like 1; SAR; SARD | |
| SARS | SERS; seryl-tRNA synthetase | |
| SAT | spermidine/spermine Nl -acetyltransferase; Hs.28491; SSAT | |
| SC4MOL | sterol-C4-methyl oxidase-like; DESP4; ERG25 | |
| SC4MOP | sterol-C4-methyl oxidase pseudogene; DESP4P1 | |
| SC5DL | “sterol-C5-desaturase (fungal ERG3, delta-5-desaturase)-like” | |
| SCAD-SRL | SDR-SRL; peroxisomal short-chain alcohol dehydrogenase | |
| SCCA2 | squamous cell carcinoma antigen 2 (leupin); PI11; Protease | |
| | Inhibitor(leucine-serpin) | |
| SCD | stearoyl-CoA desaturase (delta-9-desaturase) | |
| SCDP | stearoyl-CoA desaturase (delta-9-desaturase) pseudogene | |
| SCN4A | “HYKPP; HYPP; hyperkalemic periodic paralysis (Gamstorp disease, | |
| | adynamia episdica hereditaria); sodium channel, voltage-gated, type IV, | |
| | alpha polypeptide” | |
| SCN8A | “MED; sodium channel, voltage-gated, type VIII, alpha polypeptide; | |
| | motor endplate disease” | |
| SCO1 | “SCOD1; SCO (cytocbrome oxidase deficient, yeast) homolog 1” | |
| SCO2 | “SCO1L; SCO (cytochrome oxidase deficient, yeast) homolog 2” | |
| SDHA | “SDH2; succinate dehydrogenase complex, subunit A, flavoprotein (Fp); | |
| | Hs.469; FP” | |
| SDHB | “SDH1; succinate dehydrogenase complex, subunit B, iron sulfur (IP); | |
| | Hs.64; IP; SDH” | |
| SDHC | “succinate dehydrogenase complex, subunit C, integral membrane | |
| | protein, 15 kD” | |
| SDHD | “PGL1; succinate dehydrogenase complex, subunit D, integral mem- | |
| | brane protein; paraganglioma or familial glomus tumors 1; PGL” | |
| SDR1 | short-chain dehydrogenase/reductase 1; RSDR1 | |
| SDS | serine dehydratase; Hs.76751; L-SERINE DEHYDRATASE; SDH | |
| SEL | SEL-PEN; Selenophosphate synthetase | |
| SETMAR | SET domain and mariner transposase fusion gene | |
| SGK | serum/glucocorticoid regulated kinase; SGK1 | |
| SGK2 | serum/glucocorticoid regulated kinase 2; H-SGK2 | |
| SGKL | SGK2; serum/glucocorticoid regulated kinase-like; SGK3 | |
| SGPL1 | SPL; sphingosine-1-phosphate lyase 1 | |
| SGSH | N-sulfoglucosamine sulfohydrolase (sulfamidase); HSS | |
| SH2D1A | “LYP; SH2 domain protein 1A, Duncan's disease (lymphoproliferative | |
| | syndrome); XLP; IMD5; MTCP1; lymphoproliferative syndrome; SAP; | |
| | DSHP; EBVS; XLPD; Duncan disease” | |
| SHMT1 | serine hydroxymethyltransferase 1 (soluble); Hs.8889; CSHMT; | |
| | cytoplasmic serine hydroxymethyltransferase | |
| SHMT1P | serine hydroxymethyltransferase 1 (soluble) pseudogene | |
| SHMT2 | SHMT; senne hydroxymethyltransferase 2 (mitochondrial) | |
| SI | Hs.2996; sucrase-isomaltase | |
| SIASD | SD; sialic acid storage disease; Salla Disease | |
| SIAT1 | “Hs.2554; sialyltransferase 1 (beta-galactoside alpha-2,6-sialytransferase)” | |
| SIAT2 | sialyltransferase 2 (monosialoganglioside sialyltransferase) | |
| SIAT3 | “SIAT3-PEN; sialyltransferase 3 (Gal beta 1,3 (4) Glc NAc Alpha 2,3- | |
| | sialyltransferase); ST3N” | |
| SIAT4A | “Hs.60617; sialyltransferase 4A (beta-galactosidase alpha-2,3- | |
| | sialytransferase)” | |
| SIAT4B | “sialyltransferase 4B (beta-galactosidase alpha-2,3-sialytransferase)” | |
| SIAT4C | “CG523; SIAT4; NANTA3; sialyltransferase 4C (beta-galactosidase | |
| | alpha-2,3-sialytransferase)” | |
| SIAT5 | “STZ; SAT3; sialyltransferase 5 (galactosyldiacylglycerol alpha 2,3- | |
| | sialyltransferase)” | |
| SIAT6 | “sialyltransferase 6 (N-acetyllacosaminide alpha 2,3-sialyltransferase)” | |
| SIAT7 | “sialyltransferase 7 ((alpha-N-acetylneuraminyl-2,3-beta-galactosyl-1,3)- | |
| | N-acetyl galactosaminide alpha-2,6-sialyltransferase)” | |
| SIAT8A | “SIAT8; Hs.82527; sialyltransferase 8 (alpha-N-acetylneuraminate: | |
| | alpha-2,8-sialytransferase, GD3 synthase)” | |
| SIAT8B | “STX; ST8SIA-II; sialyltransferase 8 (alpha-2,8-sialytransferase) B” | |
| SIAT8D | PST; polysialyltransferase | |
| SIAT9 | “ST3GALV; SIATGM3S; sialyltransferase 9 (CMP- | |
| | NeuAc:lactosylceramide alpha-2,3-sialyltransferase; GM3 synthase)” | |
| SIATL1 | sialyltransferase-like 1 | |
| SIP2-28 | CIB; KIP; calcium and integring binding protein (DNA-dependent | |
| | protein kinase interacting protein) | |
| SKAP55 | src kinase-associated phosphoprotein of 55 kDa | |
| SKP1A | S-phase kinase-associated protein 1A (p19A) | |
| SKP1B | S-phase kinase-associated protein 1B (p19B) | |
| SKP2 | S-phase kinase-associated protein 2 (p45) | |
| SLC23A1 | “SVCT1; YSPL3; solute carrier family 23 (nucleobase transporters), | |
| | member 1” | |
| SLC25A16 | GDA; ML7; solute carrier family 25 (mitochondrial carrier; Graves | |
| | disease autoantigen) member 16 | |
| SLC25A20 | “CACT; solute carrier family 25 (carnitine/acylcarnitine translocase), | |
| | member 20; carnitine/acylcarnitine translocase; CAC” | |
| SLC25A20P | “CACTP; solute carrier family 25 (carnitine/acylcarnitine translocase), | |
| | member 20 pseudogene; camitine/acylcarnitine translocase pseudogene ” | |
| SLK | “SNF1 (sucrose nonfermenting, yeast, homolog)-like kinase” | |
| SLPI | secretory leukocyte protease inhibitor (antileukoproteinase); HUSI-I | |
| SMA@ | “SMA; spinal muscular atrophy (Werdnig-Hoffmann disease, Kugelberg- | |
| | Welander disease)” | |
| SMARCA3 | “SNF2L3; SNF2 (sucrose nonfermenting, yeast, homolog)-like 3; | |
| | SWI/SNF related, matrix associated, actin dependent regulator of | |
| | chromatin, subfamily a, member 3; HLTF; HIP116; helicase-like | |
| | transcription factor” | |
| SMARCB1 | “SNFSL1; SWI/SNF related, matrix associated, actin dependent | |
| | regulator of chromatin, subfamily b, member 1; INI1; SNF5 (sucrose | |
| | nonfermenting, yeast, homolog)-like 1 (integrase interactor 1); Snrl; | |
| | BAF47; hSNFS; Sfh1p” | |
| SMPD1 | “sphingomyelin phosphodiesterase 1, acid lysosomal (acid | |
| | sphingomyelinase); Hs.77813; Niemann-Pick disease” | |
| SMPD2 | “sphingomyelin phosphodiesterase 2, neutral membrane (neutral | |
| | sphingomyelinase); nSMase” | |
| SMS | spermine synthase; SpS | |
| SNCA | “PARK 1; synuclein, alpha (non A4 component of amyloid precursor); | |
| | Parkinson disease, familial 1; Hs.76930; NACP; PD1” | |
| SNK | serum-inducible kinase | |
| SOAT1 | SOAT; Hs.172; STAT; ACAT; sterol O-acyltransferase (acyl-Coenzyme | |
| | A: cholesterol acyltransferase); ACAT-1 | |
| SOAT2 | sterol O-acyltransferase 2; ACAT2; ARGP2; sterol O-acyltransferase 2 | |
| SOD1 | “superoxide dismutase 1, soluble (amyotrophic lateral sclerosis 1 | |
| | (adult)); Hs.75428; ALS; ALS1” | |
| SOD2 | “Hs.73830; superoxide dismutase 2, mitochondrial” | |
| SOD3 | “Hs.2420; superoxide dismutase 3, extracellular” | |
| SORD | Hs.878; sorbitol dehydrogenase | |
| SP-22 | thioreductase-dependent peroxide reductase SP-22 | |
| SPAM1 | “sperm adhesion molecule 1 (PH-20 hyaluronidase, zona pellucida | |
| | binding); PH-20; HYAL3” | |
| SPC18 | signal peptidase complex (18 kD) | |
| SPHAR | s-phase response gene | |
| SPHK1 | sphingosine kinase 1 | |
| SPINK1 | “Hs.46262; serine protease inhibitor, Kazal type 1” | |
| SPINK2 | “HUSI-II; serine protease inhibitor, Kazal type 2 (acrosin-trypsin | |
| | inhibitor)” | |
| SPINT1 | “serine protease inhibitor, Kunitz type 1” | |
| SPINT2 | “KOP; HAI-2; serine protease inhibitor, Kunitz type, 2” | |
| SPINT3 | “HKIB9; serine protease inhibitor, Kunitz type, 3” | |
| SPR | “sepiapterin reductase (7,8-dihydrobiopterin:NADP+ oxidoreductase)” | |
| SPS2 | selenophosphate synthetase 2 | |
| SPTI | LCB1; serine palmitoyltransferase subunit I | |
| SPUVE | “serine protease, umbilical endothelium” | |
| SQLE | squalene epoxidase | |
| SRD5A1 | “steroid-5-alpha-reductase, alpha polypeptide 1 (3-oxo-5 alpha-steroid | |
| | delta 4-dehydrogenase alpha 1); Hs.552” | |
| SRD5A2 | “steroid-5-alpha-reductase, alpha polypeptide 2 (3-oxo-5 alpha-steroid | |
| | delta 4-dehydrogenase alpha 2); Hs.1989” | |
| SRD5AP1 | “steroid-5-alpha-reductase, alpha polypeptide pseudogene 1 (3-oxo-5 | |
| | alpha-steroid delta 4-dehydrogenase alpha pseudogene)” | |
| SRD5BP1 | “steroid-5-beta-reductase, beta polypeptide pseudogene 1” | |
| SRM | Hs.76244; SRML1; spermidine synthase | |
| SRML2 | spermidine synthase-like 2 | |
| SRMS | SRM; src-related kinase lacking C-terminal regulatory tyrosine and N- | |
| | terminal myristylation sites | |
| SRPK1 | SFRS protein kinase 1; SFRSK1 | |
| SRPK2 | SFRS protein kinase 2; SFRSK2 | |
| ST3GALVI | “alpha2,3-sialyltransferase” | |
| STAT3 | signal transducer and activator of transcription 3 (acute-phase response | |
| | factor); Hs.1618; APRF | |
| STE | “sulfotransferase, estrogen-preferring; EST” | |
| STGD2 | Stargardt disease 2 (autosomal dominant) | |
| STGD3 | Stargardt disease 3 (autosomal dominant) | |
| STGD4 | Stargardt disease 4 (autosomal dominant) | |
| STHM | sialyltransferase | |
| STK10 | serine/threonine kinase 10; LOK | |
| STK11 | serine/threonine kinase 11 (Peutz-Jeghers syndrome); PJS; LKB1 | |
| STK12 | AIK2; ARK2; ATM-1; serine/threonine kinase 12 | |
| STK13 | serine/threonine kinase 13 (aurora/IPL1-like) | |
| STK14A | serine/threonine kinase 14 alpha; p70S6k | |
| STR15 | serine/threonine kinase 15; BTAK; serine/threonine kinase 15 | |
| STK16 | MPSK; PKL12; serine/threonine kinase 16 | |
| STK17A | DRAK1; serine/threonine kinase 17a (apoptosis-inducing) | |
| STK17B | DRAK2; serine/threonine kinase 17b (apoptosis-inducing) | |
| STK18 | serine/threonine kinase 18 | |
| STK19 | serine/threonine kinase 19; D6S974E; D6S60; D6S60E; RP1; G11 | |
| STK2 | Hs.1087; serine/threonine kinase 2 | |
| STK3 | “serine/threonine kinase 3 (Ste20, yeast homolog); MST2; KRS1” | |
| STK4 | “serine/threonine kinase 4 (Ste20, yeast homolog); MST1; KRS2” | |
| STK6 | serine/threonine kinase 6; aurora IPL1-like kinase; BTAK; AIK | |
| STK6P | serine/threonine kinase 6 pseudogene; STK6P1 | |
| STK9 | serine/threonine kinase 9 | |
| STS | “ARSC1; ARSC; Hs.79876; arylsulfatase C, isozyme S; steroid sulfatase | |
| | (microsomal)” | |
| STSP | steroid sulfatase (microsomal) pseudogene | |
| SUCLA2 | “succinate-CoA ligase, ADP-forming, beta subunit” | |
| SUCLG1 | “SUCLA1; succinate-CoA ligase, GDP-forming, alpha subunit” | |
| SUCLG2 | “succinate-CoA ligase, GDP-forming, beta subunit” | |
| SULT | sulfotransferase | |
| SULT1A1 | “STP1; sulfotransferase family 1A, phenol-preferring, member 1; STP; | |
| | P-PST; sulfotransferase, phenol-preferring 1” | |
| SULT1A2 | “STP2; sulfotransferase family 1A, phenol-preferring, member 2; | |
| | sulfotransferase, phenol-preferring 2; HAST4” | |
| SULT1A3 | “STM; sulfotransferase family 1A, phenol-preferring, member 3; | |
| | TL-PST; sulfotransferase, monoamine-preferring” | |
| SULT1C1 | sulfotransferase 1C1 | |
| SULT1C2 | SULT1C sulfotransferase | |
| SULT2A1 | “STD; sulfotransferase family 2A, dehydroepiandrosterone (DHEA)- | |
| | preferring, member 1; Hs.81884; DHEA-ST; sulfotransferase, | |
| | dehydroepiandrosterone (DHEA)-preferring” | |
| SULT2B1 | “sulfotransferase family 2B, member 1; HSST2” | |
| SUOX | sulfite oxidase | |
| SURB7 | “SRB7; SRB7 (suppressor of RNA polymerase B, yeast) homolog” | |
| SYK | Hs.74101; spleen tyrosine kinase | |
| SYNGAP | “synaptic Ras GTPase activating protein, 135-kD, rat, homolog of” | |
| SYNJ1 | synaptojanin 1; inositol 5′-phosphatase (synaptojanin 1); INPP5G | |
| SYNJ2 | synaptojanin 2; inositol phosphate 5′-phosphatase 2 (synaptojanin 2); | |
| | INPP5H | |
| TACTILE | “T cell activation, increased late expression” | |
| TADA3L | “ADA3; transcriptional adaptor 2 (ADA2, yeast homolog)-3 like (PCAF | |
| | histone acetylase complex)” | |
| TAF1A | “SL1; TAFI48; TATA box binding protein (TBP)-associated factor, RNA | |
| | polymerase I, A, 48 kD” | |
| TAF1B | “SL1; TAFI63; TATA box binding protein (TBP)-associated factor, RNA | |
| | polymerase I, B, 63 kD” | |
| TAF1C | “SL1; TAFI95; TAFI110; TATA box binding protein (TBP)-associated | |
| | factor, RNA polymerase I, C, 110 kD” | |
| TAF2A | “CCG1; BA2R; TATA box binding protein (TBP)-associated factor, | |
| | RNA polymerase II, A, 250 kD; CCGS; NSCL2; TAFII250; BALB/c | |
| | 3T3 ts2 temperature sensitivity complementing; cell cycle, G1 | |
| | phase defect, | |
| | (transcription factor TFIID p250 polypeptide)” | |
| TAF2B | “TATA box binding protein (TBP)-associated factor, RNA polymerase II, | |
| | B, 150 kD; TAFII150” | |
| TAF2C1 | “TAF2C; TATA box binding protein (TBP)-associated factor, RNA | |
| | polymerase II, C1, 130 kD; TAFII130; TAFII135” | |
| TAF2C2 | “TATA box binding protein (TBP)-associated factor, RNA polymerase II, | |
| | C2, 105 kD; TAFII105” | |
| TAF2D | “TATA box binding protein (TBP)-associated factor, RNA polymerase II, | |
| | D, 100 kD; TAFII100” | |
| TAF2E | “TATA box binding protein (TBP)-associated factor, RNA polymerase II, | |
| | B, 70/85 kD; TAFII70; TAFII85” | |
| TAF2F | “TAFII55; TATA box binding protein (TBP)-associated factor, RNA | |
| | polymerase II, F, 55 kD” | |
| TAF2G | “TATA box binding protein (TBP)-associated factor, RNA polymerase II, | |
| | G, 32 kD; TAFII31; TAFII32” | |
| TAF2H | “TATA box binding protein (TBP)-associated factor, RNA polymerase II, | |
| | H, 30 kD; TAF2A; TAFII30” | |
| TAF2I | “TATA box binding protein (TBP)-associated factor, RNA polymerase II, | |
| | I, 28 kD; TAFII28” | |
| TAF2J | “TATA box binding protein (TBP)-associated factor, RNA polymerase II, | |
| | J, 20 kD; TAFII20” | |
| TAF2K | “TATA box binding protein (TBP)-associated factor, RNA polymerase II, | |
| | K, 18 kD; TAFII18” | |
| TAF3A | “TAFIII134 TATA box binding protein (TBP)-associated factor, RNA | |
| | polymerase III, A, 134 kD” | |
| TAF3B | “TAFIII120; TATA box binding protein (TBP)-associated factor, RNA | |
| | polymerase III, B, 120 kD” | |
| TAF3D | “TAFIII80; TATA box binding protein (TBP)-associated factor, RNA | |
| | polymerase III, D, 80 kD” | |
| TALDO1 | transaldolase 1 | |
| TALDOP1 | TALDO; transaldolase pseudogene 1; Hs.77290; TAL-H | |
| TAO1 | KIAA0881; thousand and one amino acid protein kinase | |
| TARS | Hs.84131; threonyl-tRNA synthetase | |
| TAT | Hs.2999; tyrosine aminotransferase | |
| TBXAS1 | “thromboxane A synthase 1 (platelet, cytochrome P450, subfamily V); | |
| | CYP5A1; CYP5” | |
| TDD | “testicular 17,20-desmolase deficiency” | |
| TDG | thymine-DNA glycosylase | |
| TDO2 | “tryptophan 2,3-dioxygenase” | |
| TDO2L1 | “tryptophan 2,3-dioxygenase-like 1” | |
| TDPGD | “dTDP-D-glucose 4,6-dehydratase” | |
| TDPX1 | “thioredoxin-dependent peroxide reductase 1 (thiol-specific antioxidant | |
| | 1, natural killer-enhancing factor B); PRP; NKEFB” | |
| TDPX2 | “PAGB; thioredoxin-dependent peroxide reductase 2 (thiol-specific | |
| | antioxidant 2, proliferation-associated gene B)” | |
| TEC | tec protein tyrosine kinase; Hs.89656; PSCTK4 | |
| TEK | “VMCM; TEK tyrosine kinase, endothelial (venous malformations, | |
| | multiple cutaneous and mucosal); TEK tyrosine kinase, endothelial; | |
| | TIE2; VMCM1; venous malformations, multiple cutaneous and mucosal” | |
| TEP1 | telomerase-associated protein 1, telomerase protein component 1, TP1, | |
| | TLP1 | |
| TERC | telomerase RNA component; hTR | |
| TERT | telomerase reverse transcriptase; TRT; TP2; TCS1; hEST2 | |
| TESK1 | testis-specific kinase 1 | |
| TESK2 | testis-specific kinase 2 | |
| TGFBR1 | “ALK-5; ACVRLK4; transforming growth factor, beta receptor I (activin | |
| | A receptor type II-like kinase, 53 kD)” | |
| TGM1 | “transglutaminase 1 (K polypeptide epidermal type I, protein-glutamine- | |
| | gamma-glutamyltransferase); Hs.22; ICR2; TGASE; ichthyosis congenita | |
| | II, non-erythromatous lamellar ichthyosis” | |
| TGM2 | “transglutaminase 2 (C polypeptide, protein-glutamine-gamma- | |
| | glutamyltransferase)” | |
| TGM3 | “Hs.2022; transglutaminase 3 (E polypeptide, protein-glutamine-gamma- | |
| | glutamyltransferase)” | |
| TGM4 | Hs.2387; transglutaminase 4 (prostate) | |
| TGM5 | TGX; TGMX; transglutaminase 5 | |
| TH | Hs.89849; tyrosine hydroxylase; Hs.2031; Hs.89780 | |
| THOP1 | thimet oligopeptidase 1 | |
| THOP2 | thimet oligopeptidase 2 | |
| TIE | Hs.78824; JTK14; tyrosine kinase with immunoglobulin and epidermal | |
| | growth factor homology domains; TIE1 | |
| TIM17 | TIM17A; preprotein translocase | |
| TIM17B | JM3; inner mitochondrial membrane preprotein translocase | |
| TIM44 | mitochondrial inner membrane translocase | |
| TIMM8A | “DFN1; translocase of inner mitochondrial membrane 8 (yeast) homolog | |
| | A; deafness, X-linked 1, progressive; DXS1274E; DDP; MTS; deafness | |
| | 1, progressive; Mohr-Tranebjaerg syndrome; deafuess/dystonia peptide” | |
| TIMP1 | “Hs.1417; EPO; TIMP; CLGI; tissue inhibitor of metalloproteinase 1 | |
| | (erytbroid potentiating activity, collagenase inhibitor)” | |
| TIMP2 | Hs.1795; tissue inhibitor of metalloproteinase 2 | |
| TIMP3 | “SED; tissue inhibitor of metalloproteinase 3 (Sorsby fundus dystrophy, | |
| | pseudoinflammatory)” | |
| TIMP4 | tissue inhibitor of metalloproteinase 4 | |
| TK1 | “thymidine kinase 1, soluble; Hs.2033” | |
| TK2 | “thymidine kinase 2, mitochondrial” | |
| TKT | Hs. 89643; transketolase (Wernicke-Korsakoff syndrome) | |
| TKTL1 | transketolase-like 1; TKR; transketolase-related gene; TKR-PEN; TKT; | |
| | TKT2 | |
| TLK2 | tousled-like kinase 2; serine/threonine kinase; PKU-alpha | |
| TLSP | “protease, serine, trypsin-like” | |
| TMPRSS2 | “transmembrane protease, serine 2; PRSS10” | |
| TMPRSS3 | “transmembrane protease, serine 3” | |
| TNK1 | “tyrosine kinase, non-receptor, 1” | |
| TNKS | “tankyrase, TRF1-interacting ankyrin-related ADP-ribose polymerase; | |
| | PARPL; TIN1; TINF1” | |
| TOM34 | HTOM34P; outer mitochondrial membrane translocase (34 kD) | |
| TOP1 | Hs.317; topoisomerase (DNA) I | |
| TOP1P1 | topoisomerase (DNA) I pseudogene 1 | |
| TOP1P2 | topoisomerase (DNA) I pseudogene 2 | |
| TOP2A | Hs.3378; TOP2; topoisomerase (DNA) II alpha (170 kD) | |
| TOP2B | Hs.75248; topoisomerase (DNA) II beta (180 kD) | |
| TOP3 | topoisomerase (DNA) III | |
| TOP3B | topoisomerase (DNA) III beta | |
| TPH | TPRH; tryptophan hydroxylase (tryptophan 5-monooxygenase) | |
| TPI1 | triosephosphate isomerase 1 | |
| TPMT | thiopurine S-methyltransferase; Hs.85291; Hs.74021 | |
| TPO | thyroid peroxidase; Hs.2041 | |
| TPP2 | Hs.1117; tripeptidyl peptidase II | |
| TPS1 | “Hs.73834; tryptase, alpha” | |
| TPS2 | “Hs.99917; tryptase, beta (tryptase II); Hs.1127; Hs.96059” | |
| TPST1 | tyrosylprotein sulfotransferase 1 | |
| TPST2 | tyrosylprotein sulfotransferase 2 | |
| TPTE | transmembrane phosphatase with tensin homology | |
| TR | TR3; TRXR2; thioredoxin reductase beta | |
| TRAD | DUET; serine/threonine kinase with Dbl- and pleckstrin homology | |
| | domains | |
| TREH | trehalase (brush-border membrane glycoprotein); TRE; TREA | |
| TREX1 | “three prime repair exonuclease 1; deoxyribonuclease III, dnaQ/mutD | |
| | (E. coli)-like; DRN3” | |
| TREX2 | Three prime repair exonuclease 2 | |
| TRF4 | LAK-1; TRF4-1; topoisomerase-related function protein 4 | |
| TST | Hs.74097; thiosulfate sulfurtransferase (rhodanese) | |
| TTF1 | “transcription termination factor, RNA polymerase I; Hs.89853” | |
| TTF2 | “transcription termination factor, RNA polymerase II; HUF2; transcrip- | |
| | tion termination factor, RNA polymerase II” | |
| TTK | Hs.2052; TTK protein kinase | |
| TXK | TXK tyrosine kinase; Hs.29877; TKL; PSCTK5 | |
| TXNRD1 | TXNR; thioredoxin reductase 1; Hs.13046 | |
| TYK2 | Hs.75516; JTK1; tyrosine kinase 2 | |
| TYMS | Hs.82962; TS; thymidylate synthetase | |
| TYP1 | TYP1-PEN; threonine-tyrosine phosphatase 1 | |
| TYR | Hs.2053; OCAIA; tyrosinase (oculocutaneous albinism IA) | |
| TYRL | tyrosinase-like | |
| TYRO3 | RSE; Tyro3 protein tyrosine kinase; Tyro3 protein tyrosine kinase (sea- | |
| | related receptor tyrosine kinase); Hs.301; Dtk; Brt; Tif; Sky | |
| TYRO3P | TYRO3P protein tyrosine kinase pseudogene | |
| TYRO4 | TYRO4 protein tyrosine kinase | |
| TYROBP | DAP12; KARAP; TYRO protein tyrosine kinase binding protein | |
| TYRP1 | Hs.75219; CAS2; TYRP; tyrosinase-related pprotein 1 | |
| U5-200-KD | “U5 snRNP-specific protein, 200 kDa (DEXH RNA helicase family)” | |
| UBE3A | “ubiquitin protein ligase E3A (human papilloma virus E6-associated | |
| | protein, Angelman syndrome); E6-AP; EPVE6AP; AS; Angelman | |
| | syndrome” | |
| UBE3AP1 | ubiquitin protein ligase E3A pseudogene 1 | |
| UBE3AP2 | ubiquitin protein ligase E3A pseudogene 2 | |
| UBR1 | “ubiquitin-protein ligase e3 component, N-recognin” | |
| UBTF | “UBF; upstream binding transcription factor, RNA polymerase I” | |
| UCHH2 | ubiquitin carboxyl-terminal esterase H2 (ubiquitin thiolesterase) | |
| UCHL1 | ubiquitin carboxyl-terminal esterase L1 (ubiquitin thiolesterase) | |
| UCHL2 | ubiquitin carboxyl-terminal esterase L2 (ubiquitin thiolesterase) | |
| UCHL3 | ubiguitin carboxyl-terminal esterase L3 (ubiquitin thiolesterase) | |
| UGCG | UDP-glucose ceramide glucosyltransferase | |
| UGDH | UDP-glucose dehydrogenase | |
| UGP1 | UDP-glucose pyrophosphorylase 1 | |
| UGP2 | UDP-glucose pyrophosphorylase 2 | |
| UGT1 | UGT1A1; UDP glycosyltransferase 1; GNT1 | |
| UGT2A1 | “UDP glycosyltransferase 2 family, polypeptide A1” | |
| UGT2B | “UGT2; UGT2B@; UDP-glucuronosyltransferase 2 family, polypeptide | |
| | B; UDP-glucuronosyltransferase 2 family, polypeptide B gene cluster” | |
| UGT2B10 | “UDP glycosyltransferase 2 family, polypeptide B10” | |
| UGT2B11 | “UDP glycosyltransferase 2 family, polypeptide B11” | |
| UGT2B15 | “UDP glycosyltransferase 2 family, polypeptide B15; UGT2B8” | |
| UGT2B17 | “UDP glycosyltransferase 2 family, polypeptide B17” | |
| UGT2B4 | “UDP glycosyltransferase 2 family, polypeptide B4; UGT2B11” | |
| UGT2B7 | “UDP glycosyltransferase 2 family, polypeptide B7; UGT2B9” | |
| UGT8 | CGT; UDP glycosyltransferase 8 (UDP-galactose ceramide galactosyl | |
| | transferase); Hs.57700 | |
| ULK1 | unc-51 (C. elegans)-like kinase 1 | |
| UMPH2 | uridine 5′-monophosphate phosphohydrolase 2 | |
| UMPK | uridine monophosphate kinase | |
| UMPS | Hs.2057; uridine monophosphate synthetase (orotate phosphoribosyl | |
| | transferase and orotidine-5′-decarboxylase) | |
| UNG | Hs.78853; DGU; UDG; uracil-DNA glycosylase; Hs.3041 | |
| UNG2 | uracil-DNA glycosylase 2 | |
| UNGP1 | UNGP 1-PEN; uracil-DNA glycosylase pseudogene 1 | |
| UNGP2 | UNGP2-PEN; uracil-DNA glycosylase pseudogene 2 | |
| UOX | urate oxidase | |
| UP | uridine phosphorylase | |
| UQCR | ubiquinol-cytochrome c reductase (6.4 kD) subunit | |
| UQCRB | ubiquinol-cytochrome c reductase binding protein; Hs.1926; UQBP; QP-C | |
| UQCRBP1 | ubiquinol-cytochrome c reductase binding protein pseudogene 1 | |
| UQCRBP2 | ubiquinol-cytochrome c reductase binding protein pseudogene 2 | |
| UQCRC1 | D3S3191; Hs.99878; ubiquinol-cytochrome c reductase core protein I; | |
| | Hs.75164 | |
| UQCRC2 | ubiquinol-cytoclirome c reductase core protein II | |
| UQCRFS1 | “RIS1; ubiquinol-cytochrome c reductase, Rieske iron-sulfur polypeptide | |
| | 1” | |
| UQCRFSL1 | “ubiquinol-cytochrome c reductase, Rieske iron-sulfur polypeptide-like 1” | |
| UQCRH | ubiguinol-cytochrome c reductase hinge protein | |
| UROD | Hs.78601; uroporphyrinogen decarboxylase | |
| UROS | Hs.75593; uroporphyrinogen III synthase (congenital erythropoietic | |
| | porphyria) | |
| USP1 | ubiquitin specific protease 1 | |
| USP11 | UHX1; ubiquitin specific protease 11 | |
| USP13 | ISOT-3; ubiquitin specific protease 13 (isopeptidase T-3) | |
| USP14 | TGT; ubiquitin specific protease 14 (tRNA-guanine transglycosylase) | |
| USP15 | KIAA0529; ubiquitin specific protease 15 | |
| USP16 | UBP-M; ubiquitin specific protease 16; UBPM; UBP-M | |
| USP18 | ubiguitin specific protease 18 | |
| USP19 | KIAA0891; ubiquitin specific protease 19 | |
| USP2 | ubiguitin specific protease 2; UBP41 | |
| USP20 | KIAA1003; ubiquitin specific protease 20 | |
| USP21 | USP23; ubiquitin specific protease 21; USP16 | |
| USP25 | ubiquitin specific protease 25; USP21 | |
| USP3 | ubiquitin specific protease 3 | |
| USP4 | “UNP; ubiquitin specific protease, proto-oncogene; Unph” | |
| USP5 | ubiquitin specific protease 5 (isopeptidase T); IsoT; ISOT-1 | |
| USP6 | HRP1; ubiquitin specific protease 6 (Tre-2 oncogene); TRE-2; TRE17; | |
| | hyperpolymorphic gene 1 | |
| USP7 | ubiquitin specific protease 7 (herpes virus-associated); HAUSP; | |
| | herpesvirus-associated ubiquitin-specific protease | |
| USP9X | “ubiquitin specific protease 9, X chromosome (Drosophila fat facets | |
| | related); DFFRX; Drosophila fat facets related X” | |
| USP9Y | “ubiquitin specific protease 9, Y chromosome (Drosophila fat facets | |
| | related); DFFRY; Drosophila fat facets related Y” | |
| UST | uronyl 2-sulfotransferase | |
| VAKTI | LETKI; LEKTI; serine proteinase inhibitor | |
| VARS1 | VARS; valyl-tRNA synthetase 1 | |
| VARS2 | valyl-tRNA synthetase 2 | |
| VBCH | van Buchem disease; hyperostosis corticalis generalisata | |
| VLCS-H2 | very long-chain acyl-CoA synthetase homolog 2 | |
| VMD2 | vitelliform macular dystrophy (Best disease) | |
| VRK1 | vaccinia related kinase 1 | |
| VRK2 | vaccinia related kinase 2 | |
| VWFCP | von Willebrand factor-cleaving protease | |
| WARS | IFI53; tryptophanyl-tRNA synthetase; interferon-induced protein 53; | |
| | IFP53 | |
| WARS2 | tryptophanyl tRNA synthetase 2 (mitochondrial) | |
| WWP2 | AIP2; Nedd-4-like ubiquitin-protein ligase | |
| XBX1 | “xylan 1,4-beta-xylosidase 1” | |
| XDH | Hs.250; xanthine dehydrogenase | |
| XPNPEP1 | “XPNPEP; X-prolyl aminopeptidase (aminopeptidase P) 1, soluble” | |
| XPNPEP2 | “X-prolyl aminopeptidase (aminopeptidase P) 2, membrane-bound” | |
| XPNPEPL | X-prolyl aminopeptidase (aminopeptidase P)-like; pepP | |
| XRN2 | 5′-3′ exoribonuclease 2 | |
| XWNPEP | X-tryptophanyl aminopeptidase (aminopeptidase W) | |
| XYLB | xylulokinase (H. influenzae) homolog | |
| YARS | YTS; YRS; TYRRS; tyrosyl-tRNA synthetase | |
| YSK1 | SOK1; sterile 20 (oxidant stress response kinase 1; yeast Sps1/Ste20- | |
| | related kinase 1) | |
| YVH1 | S. cerevisiae YVH1 protein-tyrosine phosphatase ortholog | |
| YWHAA | “tyrosine 3-monooxygenase/tryptophan 5-monooxygenase activation | |
| | protein, alpha polypeptide” | |
| YWHAB | “Hs.82140; tyrosine 3-monooxygenase/tryptophan 5-monooxygenase | |
| | activation protein, beta polypeptide” | |
| YWHAD | “tyrosine 3-monooxygenase/tryptophan 5-monooxygenase activation | |
| | protein, delta polypeptide” | |
| YWHAE | “tyrosine 3-monooxygenase/tryptophan 5-monooxygenase activation | |
| | protein, epsilon polypeptide; 14-3-3 epsilon” | |
| YWHAG | “tyrosine 3-monooxygenase/tryptophan 5-monooxygenase activation | |
| | protein, gamma polypeptide” | |
| YWHAH | “Hs.75544; YWHA1; tyrosine 3-monooxygenase/tryptophan 5- | |
| | monooxygenase activation protein, eta polypeptide” | |
| YWHAQ | “tyrosine 3-monooxygenase/tryptophan 5-monooxygenase activation | |
| | protein, theta polypeptide; HS1; tyrosine 3-monooxygenase/tryptophan | |
| | 5-monooxygenase activation protein, theta polypeptide” | |
| YWHAZ | “Hs.75103; tyrosine 3-monooxygenase/tryptophan 5-monooxygenase | |
| | activation protein, zeta polypeptide” | |
| ZAP70 | SRK; zeta-chain (TCR) associated protein kinase (70 kD); syk-related | |
| | tyrosine kinase | |
| ZMPSTE24 | “STE24; STE24P; FACE-1; zinc metalloproteinase, STE24 (yeast, | |
| | homolog)” | |
| ZRK | “zona pellucida receptor tyrosine kinase, 95 kD” | |
| hMP | 1 metalloprotease 1 (pitrilysin family) | |

Alternatively, the target sequence may encode a nuclear protein such as a nucleic acid binding protein. Examples of nucleic acid binding proteins that may be utilized in the present invention are presented in Table III.

TABLE III

| Name | DNA Binding Protein Description |
|---|---|
| ALRP | ankyrin-like repeat protein; CARP; C-193; cytokine inducible nuclear |
| | protein; cardiac ankyrin repeat protein |
| APEG1 | “nuclear protein, marker for differentiated aortic smooth muscle and |
| | down-regulated with vascular injury” |
| APEX | APE; APEX nuclease (multifunctional DNA repair enzyme); REF1; |
| | HAP1; apurinic/apyrimidinic (abasic) endonuclease |
| ARNT | aryl hydrocarbon receptor nuclear translocator; Hs.47477; HIF1beta |
| ARNTL | aryl hydrocarbon receptor nuclear translocator-like; MOP3; JAP3; |
| | BMAL1 |
| B4-2 | proline-rich protein with nuclear targeting signal |
| BLZF1 | JEM1; basic leucine zipper nuclear factor 1 (JEM-1) |
| C1D | nuclear DNA-binding protein |
| C1D | nuclear DNA-binding protein |
| CHD1 | chromodomain helicase DNA binding protein 1 |
| CHD1L | CHDL; CHD1L-PENDING; chromodomain helicase DNA binding |
| | protein 1-like |
| CHD2 | chromodomain helicase DNA binding protein 2 |
| CHD3 | chromodomain helicase DNA binding protein 3; Mi-2a |
| CHD4 | chromodomain helicase DNA binding protein 4; Mi-2b |
| DAP10 | DNAX-activation protein 10 |
| DDB1 | Hs.74623; damage-specific DNA binding protein 1 (127 kD) |
| DDB2 | Hs.77602; damage-specific DNA binding protein 2 (48 kD) |
| DDX9 | “DEAD/H (Asp-Glu-Ala-Asp/His) box polypeptide 9 (RNA helicase A, |
| | nuclear DNA helicase II); NDHII” |
| DDX9 | “DEAD/H (Asp-Glu-Ala-Asp/His) box polypeptide 9 (RNA helicase A, |
| | nuclear DNA helicase II); NDHII” |
| DDXL | “nuclear RNA helicase, DECD variant of DEAD box family” |
| DEK | DEK oncogene (DNA binding); D6S231E |
| DFFA | “DNA fragmentation factor, 45 kD, alpha subunit” |
| DFFB | “DNA fragmentation factor, 40 kD, beta polypeptide (caspase-activated |
| | DNase); DNA fragmentation factor, 40 kD, beta subunit; CAD; DFF2; |
| | CPAN; DFF40; DFF-40” |
| DMC1 | “DMC1 (dosage suppressor of mck1, yeast homolog) meiosis-specific |
| | homologous recombination; DMC1H; disrupted meiotic cDNA 1 |
| | homolog; LIM15” |
| DNA2L | “DNA2 (DNA replication helicase, yeast, homolog)-like” |
| DNAH11 | “DNAHC11; dynein, axonemal, heavy chain 11” |
| DNAH12 | DHC3; HL19; HDHC3; HL-19; DNAHC3; DNAHC12; dynein heavy |
| | chain 12 |
| DNASE2 | “DNL2; deoxyribonuclease II, lysosomal; DNL; DNase II, lysosomal” |
| ENC1 | “NRPB; nuclear restricted protein, BTB domain-like (brain); PIG10; |
| | NRP/B” |
| FBRNP | heterogeneous nuclear protein similar to rat helix destabilizing protein |
| GADD45A | DDIT1; Hs.80409; GADD45; DNA-damage-inducible transcript 1 |
| GADD45G | “CR6; GADD45-GAMMA; growth arrest and DNA-damage-inducible, |
| | gamma” |
| GRLF1 | GRF-1; glucocorticoid receptor DNA binding factor 1 |
| HDGF | hepatoma-derived growth factor (high-mobility group protein 1-like); |
| | HMG1L2 |
| HIRIP4 | DNAJ; HIRA interacting protein 4 (dnaJ-like) |
| HLJ1 | DNAJW; DnaJ-like heat shock protein 40 |
| HMG1 | high-mobility group (nonhistone chromosomal) protein 1; HMG3; |
| | Hs.74570 |
| HMG1L1 | HMG1L7; high-mobility group (nonhistone chromosomal) protein 1-like |
| | 1 |
| HMGCS1 | Hs.21808; HMGCS; 3-hydroxy-3-methylglutaryl-Coenzyme A synthase 1 |
| | (soluble) |
| HMGIY | high-mobility group (nonhistone chromosomal) protein isoforms I and Y; |
| | Hs.64605; HMGI-Y; HMGI/Y |
| HNF3A | “hepatocyte nuclear factor 3, alpha” |
| HNF3B | “hepatocyte nuclear factor 3, beta” |
| HNF3G | “hepatocyte nuclear factor 3, gamma” |
| HNF4A | “TCF14; hepatic nuclear factor 4, alpha” |
| HNF4B | “hepatocyte nuclear factor 4, beta” |
| HNF4G | “hepatocyte nuclear factor 4, gamma” |
| HNF6 | hepatocyte nuclear factor 6 |
| HNF6A | “hepatocyte nuclear factor 6, alpha” |
| HRIHFB2122 | putative nuclear protein |
| HSJ1 | “heat shock protein, neuronal DNAJ-like, 1; HSPF3” |
| HSJ2 | “heat shock protein, DNAJ-like 2; HSPF4; dj-2; hdj-2” |
| ID1 | “Hs.75424; inhibitor of DNA binding 1, dominant negative helix-loop- |
| | helix protein” |
| ID2 | “inhibitor of DNA binding 2, dominant negative helix-loop-helix protein; |
| | Hs.76667” |
| ID3 | “Hs.76884; HEIR-1; inhibitor of DNA binding 3, dominant negative |
| | helix-loop-helix protein” |
| ID4 | “Hs.34853; inhibitor of DNA binding 4, dominant negative helix-loop- |
| | helix protein” |
| INSL | Insulin-like DNA sequence |
| KIAA0765 | HRIHFB2091; putative brain nuclearly-targeted protein |
| KIP2 | DNA-dependent protein kinase catalytic subunit-interacting protein 2 |
| LAF4 | lymphoid nuclear protein 4 |
| LHFP | lipoma HMGIC fusion partner |
| LHFPL1 | lipoma HMGIC fusion partner-like 1 |
| LHFPL3 | lipoma HMGIC fusion partner-like 3 |
| LHFPL4 | lipoma HMGIC fusion partner-like 4 |
| LIG1 | “Hs.1770 ligase I, DNA, ATP-dependent” |
| LIG2 | “ligase II, DNA, ATP-dependent” |
| LIG3 | “Hs.100299; ligase III, DNA, ATP-dependent” |
| LIG4 | “ligase IV, DNA, ATP-dependent” |
| LPSA | “Oncogene liposarcoma (DNA segment, single copy, expressed, probes” |
| LXR | “orphan nuclear hormone receptor, retinoid response” |
| M96 | putative DNA binding protein |
| MERR | metalloregulatory DNA-binding protein |
| MGMT | O-6-methylguanine-DNA methyltransferase; Hs.1384 |
| MNDA | Hs.3197; myeloid cell nuclear differentiation antigen |
| MPG | Hs.79396; MDG; N-methylpurine-DNA glycosylase |
| MRJ | MRJ gene for a member of the DNAJ protein family |
| NAGR1 | N-acetylglucosamine receptor 1 (thyroid); heterogenous nuclear |
| | ribonucleoprotein M4 |
| NASP | Hs.68875; nuclear autoantigenic sperm protein (histone-binding) |
| NCBP | “Hs.89750; nuclear cap binding protein, 80 kD; Hs.89563” |
| NCOA1 | nuclear receptor coactivator 1; SRC1; steroid receptor coactivator 1; |
| | NCoA-1; F-SRC-1 |
| NCOA3 | nuclear receptor coactivator 3; AIB1; ACTR; RAC3; p/CIP; CAGH16; |
| | TNRC16; TRAM-1; amplified in breast cancer 1 |
| NCOA4 | nuclear receptor coactivator 4; RFG; ELE1; ARA70 |
| NCOR1 | nuclear receptor co-repressor 1; N-CoR; TRAC1; hN-CoR; KIAA1047; |
| | hCIT529I10 |
| NCOR2 | nuclear receptor co-repressor 2; SMRT; CTG26; SMRTE; TNRC14; |
| | TRAC-1 |
| NCYM | DNA-binding transcriptional activator |
| NDP52 | nuclear domain 10 protein |
| NDR | “NDR-LSB; serine/threonine kinase, nuclear Dfnb2-related (Drosophila) |
| | homolog” |
| NFAT5 | nuclear factor of activated T-cells 5; TONEBP; KIAA0827 |
| NFATC1 | “nuclear factor of activated T-cells, cytoplasmic 1; NF-ATC” |
| NFATC2 | “NF-ATP; nuclear factor of activated T-cells, cytoplasmic 2” |
| NFATC3 | “NFAT4; NFATX; nuclear factor of activated T-cells, cytoplasmic 3” |
| NFATC4 | “NFAT3; nuclear factor of activated T-cells, cytoplasmic 4” |
| NFATC5 | “nuclear factor of activated T-cells, cytoplasmic 5” |
| NFE2 | “NF-E2; nuclear factor (erythroid-derived 2), 45 kD” |
| NFE2L1 | nuclear factor (erythroid-derived 2)-like 1; NRF1; LCR-F1 |
| NFE2L2 | NRF2; nuclear factor (erythroid-derived 2)-like 2 |
| NFE2L3 | NRF3; nuclear factor (erythroid-derived 2)-like 3 |
| NFIA | KIAA1439; NFI-L; nuclear factor I/A |
| NFIB | NFI-RED; nuclear factor I/B |
| NFIC | NFI; CTF; NF-I; nuclear factor I/C (CCAAT-binding transcription factor) |
| NFIL3 | “IL3BP1; nuclear factor, interleukin 3 regulated; E4BP4; NFIL3A; NF- |
| | IL3A” |
| NFIX | Hs.99929; nuclear factor I/X (CCAAT-binding transcription factor) |
| NFIXL1 | nuclear factor I/X-like 1 |
| NFIXL2 | nuclear factor I/X-like 2 |
| NFIXL3 | nuclear factor I/X-like 3 |
| NFIXL4 | NFIX; nuclear factor I/X-like 4 |
| NFIXL5 | nuclear factor I/X-like 5 |
| NFKB1 | Hs.83428; KBF1; nuclear factor of kappa light polypeptide gene enhancer |
| | in B-cells 1 (p105) |
| NFKB2 | Hs.73090; LYT-10; nuclear factor of kappa light polypeptide gene |
| | enhancer in B-cells 2 (p49/p100) |
| NFKBIA | “NFKBI; nuclear factor of kappa light polypeptide gene enhancer in B- |
| | cells inhibitor, alpha; IKBA; MAD-3” |
| NFKBIB | “nuclear factor of kappa light polypeptide gene enhancer in B-cells |
| | inhibitor, beta; IKBB; TRIP9” |
| NFKBIE | “nuclear factor of kappa light polypeptide gene enhancer in B-cells |
| | inhibitor, epsilon; IKBE” |
| NFKBIL1 | IKBL; NFKBIL; nuclear factor of kappa light polypeptide gene enhancer |
| | in B-cells inhibitor-like 1 |
| NFKBIL2 | IKBR; nuclear factor of kappa light polypeptide gene enhancer in B-cells |
| | inhibitor-like 2 |
| NFRKB | nuclear factor related to kappa B binding protein |
| NFX1 | “nuclear transcription factor, X-box binding 1” |
| NFYA | “nuclear transcription factor Y, alpha; Hs.797; HAP2; CBF-A” |
| NFYB | “nuclear transcription factor Y, beta; CBF-B” |
| NFYC | “nuclear transcription factor Y, gamma; CBF-C” |
| NIP1 | NIP1-PEN; Nuclear cap binding protein (NCBP) interacting protein-1 |
| NIP1L | “nip1 (nuclear import protein, S cerevisiae)-like” |
| NLVCF | nuclear localization signal deleted in velocardiofacial syndrome |
| NR1D1 | “EAR-1; THRAL; REV-ERBAALPHA; nuclear receptor subfamily 1, |
| | group D, member 1” |
| NR1D2 | “RVR; BD73; HZF2; EAR-1R; nuclear receptor subfamily 1, group D, |
| | member 2” |
| NR1H2 | UNR; ubiquitously-expressed nuclear receptor |
| NR1H3 | “LXRA; LXR-A; RLD-1; NR1H3-PENDING; nuclear receptor subfamily |
| | 1, group H, member 3” |
| NR1H4 | “FXR; HRR1; RIP14; NR1H4-PENDING; nuclear receptor subfamily 1, |
| | group H, member 4” |
| NR1I2 | “nuclear receptor subfamily 1, group I, member 2; PXR; SXR; SAR; |
| | BXR; ONR1; PAR2; nuclear receptor subfamily 1, group I, member 2” |
| NR1I3 | “CAR; MB67; NR1I3-PENDING; nuclear receptor subfamily 1, group I, |
| | member 3” |
| NR1I4 | “CAR2; nuclear receptor subfamily 1, group I, member 4” |
| NR2C1 | TR2; TR2 nuclear hormone receptor; TR2 |
| NR2C2 | “TR4; nuclear receptor subfamily 2, group C, member 2; TR4 nuclear |
| | hormone receptor; TAK1” |
| NR2E3 | “PNR; nuclear receptor subfamily 2, group B, member 3” |
| NR3C1 | “GRL; nuclear receptor subfamily 3, group C, member 1; Hs.75772; |
| | glucocorticoid receptor; GR; Hs.49281” |
| NR4A1 | “HMR; nuclear receptor subfamily 4, group A, member 1; hormone |
| | receptor (growth factor-inducible nuclear protein N10); TR3; Hs.1119; |
| | GFRP1; N10; NAK1; NAK-1; NGFIB; nur77” |
| NR4A2 | NURR1; nuclear receptor related 1 (transcriptionally inducible); TINUR; |
| | NOT |
| NR4A3 | “nuclear receptor subfamily 4, group A, member 3; CHN; CSMF; NOR1; |
| | MINOR” |
| NR6A1 | GCNF; germ cell nuclear factor; RTR; GCNF1 |
| NRB | “NRB-PEN; nuclear RNA-binding protein, 54 kDa” |
| NRF1 | nuclear respiratory factor 1 |
| NRIP1 | nuclear receptor interacting protein 1; RIP140 |
| NUMA1 | NUMA; nuclear mitotic apparatus protein 1 |
| NVL | nuclear VCP-like |
| OGG1 | 8-oxoguanine DNA glycosylase |
| PCBD | Hs.3192; PCD; DCOH; 6-pyruvoyl-tetrahydropterin |
| | synthase/dimerization cofactor of hepatocyte nuclear factor 1 alpha |
| | (TCF1); pterin-4-alpha carbinolamine dehydratase |
| PCBP1 | poly(rC)-binding protein 1; HNRPE1; hnRNP-E1; heterogenous nuclear |
| | ribonucleoprotein X |
| PCNA | Hs.78996; proliferating cell nuclear antigen |
| PCNAL | proliferating cell nuclear antigen-like |
| POLA | “Hs.81942; polymerase (DNA directed), alpha” |
| POLB | “Hs.1894; polymerase (DNA directed), beta” |
| POLD1 | “Hs.65383; POLD; polymerase (DNA directed), delta 1, catalytic subunit |
| | (125 kD)” |
| POLD2 | “polymerase (DNA directed), delta 2, regulatory subunit (50 kD)” |
| POLE | “polymerase (DNA directed), epsilon” |
| POLE2 | “polymerase (DNA directed), epsilon 2; DPE2” |
| POLG | “Hs.80961; polymerase (DNA directed), gamma” |
| POLG2 | “polymerase (DNA directed), gamma 2, accessory subunit; HP55; POLB; |
| | MTPOLB; polymerase (DNA directed), gamma 2, accessory subunit” |
| POLH | “polymerase (DNA directed), eta; XP-V; RAD30A” |
| POLI | RAD30B; polymerase (DNA directed) iota; RAD30 (S. cerevisiae) |
| | homolog B |
| POLQ | “polymerase (DNA-directed), theta” |
| POLR2A | polymerase (RNA) II (DNA directed) polypeptide A (220 kD); Hs.60366; |
| | POLR2; POLRA |
| POLR2B | polymerase (RNA) II (DNA directed) polypeptide B (140 kD) |
| POLR2C | Hs.79402; polymerase (RNA) II (DNA directed) polypeptide C (33 kD) |
| POLR2D | polymerase (RNA) II (DNA directed) polypeptide D |
| POLR2E | polymerase (RNA) II (DNA directed) polypeptide E (25 kD) |
| POLR2F | polymerase (RNA) II (DNA directed) polypeptide F |
| POLR2G | polymerase (RNA) II (DNA directed) polypeptide G; RPB7 |
| POLR2H | polymerase (RNA) II (DNA directed) polypeptide H |
| POLR2I | polymerase (RNA) II (DNA directed) polypeptide I (14.5 kD) |
| POLR2J | polymerase (RNA) II (DNA directed) polypeptide J (13.3 kD) |
| POLR2K | polymerase (RNA) II (DNA directed) polypeptide K (7.0 kD) |
| POLR2L | polymerase (RNA) II (DNA directed) polypeptide L (7.6 kD) |
| POLRMT | polymerase (RNA) mitochondrial (DNA directed); h-mtRPOL |
| PP32 | “acidic nuclear phosphoprotein, pp32; Putative human HLA class II |
| | associated protein I; LANP; PHAP1; ANP32; I1PP2A” |
| PP32R1 | “acidic nuclear phosphoprotein, pp32, related, 1” |
| PP32R2 | “acidic nuclear phosphoprotein, pp32, related, 2” |
| PRKDC | “HYRC1; protein kinase, DNA-activated, catalytic polypeptide; XRCC7; |
| | hyper-radiosensitivity of murine scid mutation, complementing 1; |
| | DNAPK” |
| PTB | Hs.102127; HNRPI; HNRNPI; polypyrimidine tract binding protein |
| | (heterogeneous nuclear ribonucleoprotein I); Hs.75971 |
| PUAB4 | “protein spot in 2-D gels (nuclear polypeptide, 100 kD, relative pI 6.25)” |
| RBBP2H1A | PLU-1; putative DNA/chromatin binding motif; retinoblastoma-binding |
| | protein 2 homolog 1A |
| RECQL | Hs.1536; RecQ protein-like (DNA helicase Q1-like) |
| RELA | v-rel avian reticuloendotheliosis viral oncogene homolog A (nuclear factor |
| | of kappa light polypeptide gene enhancer in B-cells 3 (p65)); NFKB3 |
| RELB | v-rel avian reticuloendotheliosis viral oncogene homolog B (nuclear factor |
| | of kappa light polypeptide gene enhancer in B-cells 3) |
| REV3L | “REV3 (yeast homolog)-like, catalytic subunit of DNA polymerase zeta; |
| | POLZ” |
| SAMSN1 | “SAM domain, SH3 domain and nuclear localisation signals, 1” |
| SATB1 | Hs.74592; special AT-rich sequence binding protein 1 (binds to nuclear |
| | matrix/scaffold-associating DNA's) |
| SATB1 | Hs.74592; special AT-rich sequence binding protein 1 (binds to nuclear |
| | matrix/scaffold-associating DNA's) |
| SCNN1D | “sodium channel, nonvoltage-gated 1, delta; dNaCh; ENaCd” |
| SIP2-28 | CIB; KIP; calcium and integring binding protein (DNA-dependent protein |
| | kinase interacting protein) |
| SON | SON DNA-binding protein; Hs.92909; DBP-5 |
| SP100 | Hs.77617; nuclear antigen Sp100 |
| SP140 | SP140-PEN; nuclear antigen Sp140 |
| SPBPBP | DNA-binding protein amplifying expression of surfactant protein B |
| SPF31 | splicing factor similar to dnaJ |
| SPN | NUDR; suppressin (nuclear deformed epidermal autoregulatory factor-1 |
| | (DEAF-1)-related) |
| SRM160 | Ser/Arg-related nuclear matrix protein (plenty of prolines 101-like) |
| SSBP | single-stranded DNA-binding protein; Hs.923 |
| SSNA1 | Sjogren's syndrome nuclear autoantigen 1; nuclear autoantigen of 14 kDa; |
| | N14; NA14 |
| TCF1 | “Hs.73888; HNF1; LFB1; transcription factor 1, hepatic; LF-B1, hepatic |
| | nuclear factor (HNE1), albumin proximal factor” |
| TCF2 | “transcription factor 2, hepatic; LF-B3; variant hepatic nuclear factor; |
| | LFB3; VHNF1; HNF1beta” |
| TCF7 | “transcription factor 7 (T-cell specific, HMG-box); Hs.100010; Hs.3002; |
| | TCF-1” |
| TCF7L1 | “transcription factor 7-like 1 (T-cell specific, HMG-box); TCF-3” |
| TCF7L2 | “transcription factor 7-like 2 (T-cell specific, HMG-box); TCF-4” |
| TDP-43 | TAR DNA-binding protein-43 |
| TIF2 | GRIP1; NCOA-2; NCOA2-PENDING; nuclear receptor coactivator 2 |
| TITF1 | NKX2A; thyroid transcription factor 1; TTF-1; NK-2 (Drosophila) |
| | homolog A (thyroid nuclear factor) |

Assembly of the inducible cassette is generally performed using standard molecular biology techniques such as restriction endonuclease digestion and ligation as described in Sambrook et al., Molecular Cloning: A Laboratory Manual, 2^{nd} Ed., Cold Spring Harbor Laboratory, 1989. In general, the inducible promoter is ligated upstream of the target insertion domain such that the promoter may induce expression of the target sequence. In addition, the selecting sequence is generally ligated in a different reading frame from the inducible promoter such that expression of the selecting sequence does not result in induction of the target.

There may be some situations in which the addition of a reporter gene is desirable. If a reporter gene is used, the positioning of the reporter gene may be different depending on the reporter gene's function. Of course, when a reporter gene is used to detect insertion of the target into the subcloning vector, the reporter gene is generally positioned such that the target insertion domain is within the reporter gene allowing the detection of an inserted target sequence by disruption of the reporter gene's expression. In contrast, when the reporter gene is used to detect insertion of the inducible construct into a mammalian cell, the reporter gene is positioned outside of the target insertion domain such that an inserted target does not disrupt expression of the reporter.

Orientation of the components that comprise the inducible cassette may further depend on the number of promoters within the cassette and the number of target sequences within the inducible cassette.

When the inducible cassette consists of one promoter, it may be operably linked to the target sequence such that it initiates transcription of the target sequence. One skilled in the art will recognize the advantages of incorporating two or more promoters within the inducible cassette.

When two or more identical target sequences are inserted into the inducible cassette, it may be desirable to have one promoter or set of tandem promoters induce expression of the entire transcript. Alternatively, when different target sequences are inserted into the same inducible cassette, it may be desirable to have at least two promoters each able to induce expression of a target individually. For example two target sequences may be inserted in different reading frames allowing the selective induction by each promoter.

The subcloning vector is a double stranded circular nucleic acid sequence able to replicate and be transcribed within a host cell and able to accept an inducible cassette. A subcloning vector preferably comprises an origin of replication site (“ori”) and an inducible cassette insertion domain. Similar to the inducible cassette, the subcloning vector may further comprise a reporter gene able to detect the insertion of the inducible cassette and a selecting gene able to select for cells expressing the subcloning vector. The type of subcloning vector used with the present invention may depend on the size of the inducible cassette to be inserted. When the subcloning vector is a plasmid the inducible cassette may be from about 0.1 kb to about 15 kb, preferably from about 0.5 kb to about 10 kb, and most preferably from 1 kb to 6 kb. Plasmids that may be used in the present invention include, for example, puc18, puc19, and pBluescript II KS. Preferably the plasmid is pc-DNA4/TO.

Endonuclease cleavage sites may be added to allow the removal or insertion of components in the subcloning vector by PCR. For example, when the same selecting sequence is present in both the inducible cassette and the subcloning vector, a cleavage site may be engineered allowing the removal of one of the selecting sequences and insertion of an alternative selecting sequence. The addition of sequences may be performed using standard PCR techniques whereby primers are designed to insert a desired endonuclease cleavage site.

Similarly, endonuclease cleavage sites within the target insertion domain may be modified such that a target sequence may be removed from and inserted into the inducible construct without removal of the inducible cassette from the subcloning vector. This allows efficient transfer of target sequences into and out of the inducible construct. For example, a cleavage site may be removed by PCR or by ligation of a DNA sequence inactivating the cleaved site.

One skilled in the art will recognize that the same strategies comprising restriction and ligation of a target cDNA sequence into an inducible cassette are applicable in inserting the inducible cassette into the subcloning vector.

In addition, more than one inducible cassette may be inserted into a subcloning vector such that a single inducible construct may express one or more target sequences. When multiple inducible cassettes are added to the subcloning vector they may be inserted in different reading frames such that each inducible cassette may be induced individually. However, one skilled in the art would recognize that induction of multiple inducible cassettes in different reading frames within the same cell would require different inducer molecules or inducing conditions allowing for selective induction. For example in one configuration an assembly protein may be required for functional activity of the target sequence. In this case the assembly protein may be inserted within a second inducible cassette allowing the assembly protein to be induced prior to induction of the target sequence. In yet another configuration, an additional inducible cassette may be inserted into the subcloning vector that encodes a growth factor or differentiation activator to enhance cell growth and promotes differentiation upon induction. Alternatively, in another configuration a reporter gene operably linked to a nuclear hormone receptor gene may be inserted into the subcloning vector such that induction produces a change in reporter activity that can be measured.

As previously discussed, the inducer molecule or induction condition allows the user to selectively induce the transcription of the target sequence. Correspondingly, the inducer molecule or induction condition may be different depending on the inducible promoter. For example, Ponasterone A is a molecule that induces the expression of a vector comprising an ecdysone promoter (Invitrogen, Carlsbad, Calif.) and tetracycline is a molecule that induces the expression of a vector comprising a tetracycline-dependent promoter (Invitrogen, Carlsbad, Calif.; Clontech, Palo Alto, Calif.).

A change in an environmental condition may also be utilized for induction. For example, heat shock promoters are known to induce transcription upon an increase in temperature. Consequently, for example by controlling the temperature of the media the user is able to control induction of a target sequence.

A repressor may be used with an inducer or may be used in place of an inducer to regulate induction. A repressor is a compound that interacts with a nucleotide sequence interfering with transcription. Therefore, induction generally occurs in the absence of a repressor. For example zinc finger proteins (“ZFPs”) are commonly used as repressors. Particularly potent ZFPs comprise a Kruppel-associated box (“KRAB”) domain (Vissing et al., FEBS Letts. 369:153-157, 1995; Beerli et al., Proc. Natl. Acad. Sci. 95:14628-14633, 1998).

A second inducible construct may encode an inducer or a repressor able to control transcription of an endogenous target. For example, an inducible expression vector encoding a regulator, such as for example VP16, FKBP or ZFP, may be used to modulate induction of the target wherein the inducer initiates transcription of the regulator and the regulator initiates transcription of the target sequence. In this configuration there may be an additional reporter within the inducible cassette or within the regulatory construct allowing the induction to be monitored between constructs.

Unlike traditional expression systems, the present invention provides an internal control because of the ability to initiate or terminate the expression of the target sequence. Therefore, modulation may be determined by comparing values collected prior to and after induction of the target sequence. In contrast, traditional methods for utilizing expression vectors generally involve transfection of an expression vector in one population of cells and transfection of a control in another population. However because there is variance in expression between populations and in stability of expression over time, modulation is difficult to measure.

The use of homologous recombination to produce the inducible target may be useful for the present invention. In this method, the endogenous promoter of an endogenous target gene is replaced with the inducible promoter of the present invention. The DNA constructs derived by homologous recombination are useful for operatively linking exogenous regulatory and structural elements to endogenous coding sequences in a way that precisely creates a novel transcriptional unit, provides flexibility in the relative positioning of exogenous regulatory elements and endogenous genes and, ultimately, enables a highly controlled system for identification of modulatory compounds.

Upon homologous recombination, the inducible regulatory sequence of the construct is integrated into a pre-selected region of the target gene in a chromosome of a cell. This region should be within 5 kb of a coding exon and more preferably within 1 kb of a coding exon for the gene of interest. The resulting new transcription unit containing the construct-derived inducible regulatory sequence alters the expression of the target gene.

According to this method, the inducible cassette may comprise 5′ and 3′ insertion adapters enabling it to be inserted into the genome of the host organism by homologous recombination using standard recombination techniques (Mansour et al., Nature 336:348, 1988; U.S. Pat. No. 6,270,989 to Treco, U.S. Pat. No. 6,242,218 to Treco, all of which are incorporated in their entireties herein by reference). In this configuration, the insertion adapters are complementary to the non-coding region of the genome where the inducible cassette is to be inserted. 5′ and 3′ adapter sequences permit homologous recombination of a desired sequence into a selected site in the host genome. These adapter sequences are homologous to (i.e., able to homologously recombine with) their respective target regions in the host genome. The adapter sequence is homologous to a pre-selected target site in the genome with which homologous recombination is to occur. It contains at least 20 (e.g., at least 50 or 100) contiguous nucleotides from the region of the target gene. By “homologous” is meant that the targeting sequence is identical or sufficiently similar to its genomic target site so that the targeting sequence and target site can undergo site-specific recombination. A small percentage of base pair mismatches is acceptable, as long as homologous recombination can occur at a useful frequency. To facilitate homologous recombination, the adapter sequence is preferably at least about 20 (e.g., 50, 100, 250, 400, or 1,000) base pairs (“bp”) long.

A circular DNA construct can employ a single adapter sequence, or two or more separate adapter sequences. A linear DNA construct may contain two or more separate targeting sequences. The target site to which a given targeting sequence is homologous can reside within an exon and/or intron of the target gene, upstream of and immediately adjacent to the target gene coding region, or upstream of and at a distance from the target gene coding region.

The use of homologous recombination to insert an inducible promoter to the regulatory region of an endogenous gene may encompass the expression of a gene which is normally silent in the cell. The use of homologous recombination may also cause the increased expression level of the endogenous gene, or may change the regulation pattern of a gene.

II. Cell Transfection

As described above, the traditional methods utilizing expression vectors require multiple transfections. In particular, the expression vector is inserted into one aliquot of cells of a sample while one or more control vectors are inserted into additional aliquots of the sample. This method is undesirable because transfection and expression efficiencies may vary significantly from sample to sample.

The methods of the present invention do not require the transfection of additional controls. Once cells have been transfected with the inducible vector construct a steady state measurement maybe obtained by assaying the cells in the absence of inducer. An activated state measurement may be made by assaying the cells in the presence of inducer and the modulation capability of a compound may be measured by assaying the cells in an activated state in the presence of the compound. Correspondingly, a steady state measurement in the presence of compound may be made following that activated state by assaying the cells once the inducer has been removed. However, one skilled in the art would recognize that careful selection may be necessary to achieve determination the desired concentration of inducer for induction during development of the assay. For example, a bulk transfection may be performed and individual cells selected to determine inducibility by measuring the target expression, either by RT-PCR/Northern blotting, western blotting, observation of a phenotypic change, or preferably all of the above. Clones with the desired expression levels are then selected, isolated and cultured to be assayed against possible modulatory compounds.

The recipient cell may be any in which the target is not endogenously active or has low or negligible activity, is able to grow from low densities, and is amenable to mass culture. Additionally, when secondary modification of the translated target is desirable such as glycosylation, the cell must be able to perform any such secondary modification. In addition, the desired recipient cell should have the appropriate signaling mechanisms for the target to initiate a phenotypic change that may be measured. For example, if the target is a GPCR, the desired cell would preferably have intact adenylyl cyclase and calcium signaling pathways. A number of recipient cells may be utilized with the present invention such as for example CHO, CHO-K1, HEK293, COS, Vero, RBL, SH-SY5Y, and U20S cells.

One factor to consider when determining whether a cell is appropriate for transfection is its endogenous expression of the target sequence. Activity may be measured using a variety of techniques such as RT-PCR, Northern analysis, and array hybridization. Suitable hosts would be those that do not have the target sequence or express it in a low level. More specifically, if a target cannot be detected by RT-PCR, it is highly unlikely that it will mediate a signaling event and therefore the cells would be desirable recipients.

Selection of clonal cell lines may be performed by growing cells from low densities and isolating colonies that desirably express the target sequence. More preferably the recipient cells are grown from single cell colonies. Recipient cells may be chosen by their ability to grow in culture to high density. In large preparations a high concentration of cells may be required. In this configuration non-adherent cells may be grown in spinner flasks and adherent cells may be grown in roller bottles.

Transfection may be performed by a variety of methods that allow vector insertion into a cell such as for example calcium phosphate and electroporation (Sambrook et al., Molecular Cloning A Laboratory Manual, 1987).

Transfected cells may be selected from those that do not express a selecting sequence by a variety of methods. Typically, when the construct comprises a selection sequence encoding resistance to a selective agent, positive cells are selected by the addition of the corresponding selective agent. Alternatively, optical assays may be used to select positive colonies when the inducible cassette comprises a reporter gene such as luciferase. In addition transfected cells may be selected using fluorescent activated cell sorting (FACS). Following selection cells are plated and grown to multicellular colonies.

Plates containing multicellular colonies are further passed into daughter plates such that there are about ten daughters per mother plate. Cells are then selected by RT-PCR and/or immunoblot analysis and target dependent responses.

III. Selection of Cells by Target-Dependent Responses

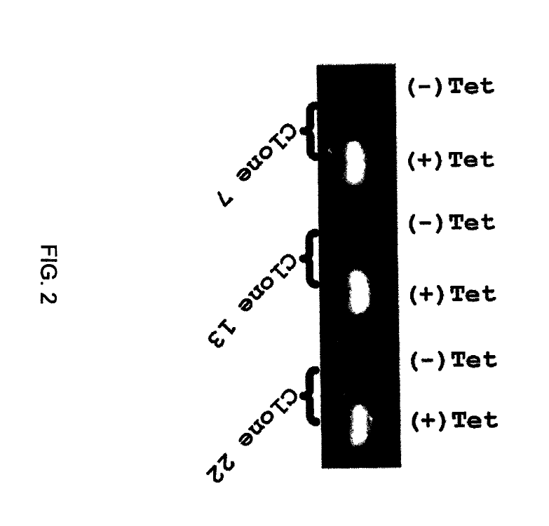

After transfection and selection of stable cell lines containing the inducible vector, the cells are tested for inducible expression of the desired mRNA. For example, upon transfection of the vector illustrated in FIG. 1 to CHO cells as described in Example 2, and subsequent selection for the presence of the plasmid, putative positive cells were tested for induction of KCNC1 mRNA expression after addition of the inducer molecule, tetracycline, following the method described in Example 3. KCNC1 mRNA was amplified by RT-PCR using primers specific for the KCNC1 gene as described in Example 3, then separated by agarose gel electrophoresis (FIG. 2). The PCR products of several clones (# 7, 13, 22) were found to express the KCNC 1 mRNA when induced.

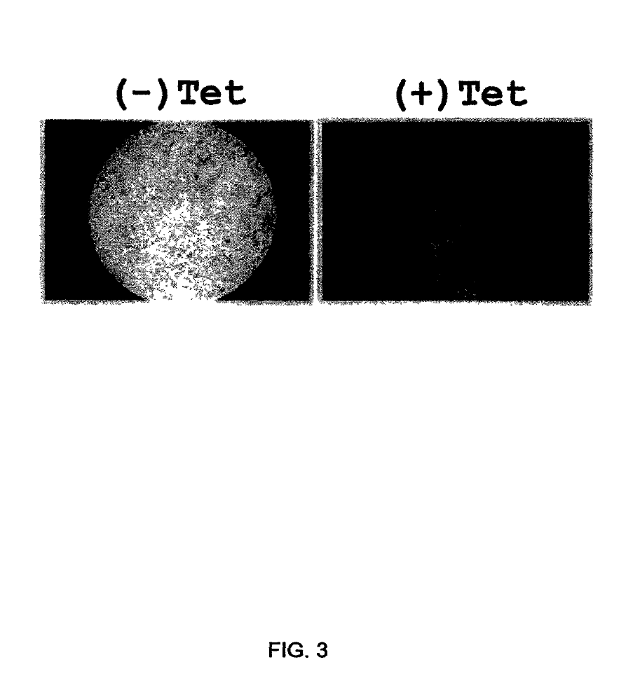

Furthermore, the inducible production of the target protein should be ensured. Using the above-described system as an example, the tetracycline-inducibility of the KCNC1 protein was determined using an immunoassay according to the method described in Example 2. Briefly, a primary antibody that recognizes the KCNC1 protein was added to the assay well. After a brief wash, the secondary antibody, conjugated to horseradish peroxidase to allow for color development, was added to the well. Upon development of the immunoassay, the tetracycline-induced well was darker than the control well (FIG. 3), indicating the presence of the KCNC1 protein. One of skill in the art will appreciate that the inducibility of any target sequence useful for the present invention can be determined in a similar manner.

Positive cells are then tested for target-dependent responses by measuring the appropriate response in both the absence and presence of the inducer in order to identify those cells expressing a functional target sequence.

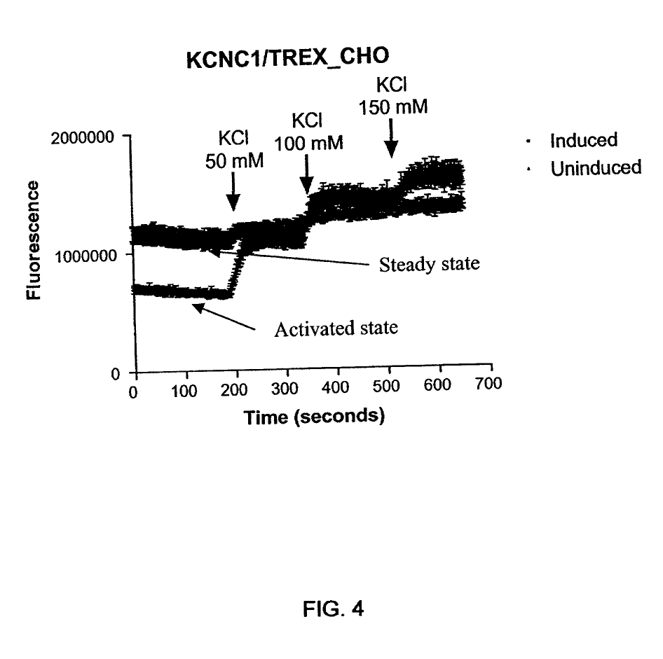

FIG. 4 demonstrates the use of a cell containing an inducible target as described herein for screening for molecules that modulate its activity. In this example, fluorescent dyes are used to assay for changes in membrane potential, essentially as described in Example 4. CHO cells induced to produce the KCNC1 target polypeptide are subsequently able to show a response (i.e. a change in fluorescence intensity of the indicator dye) when the modulator KCl is added.

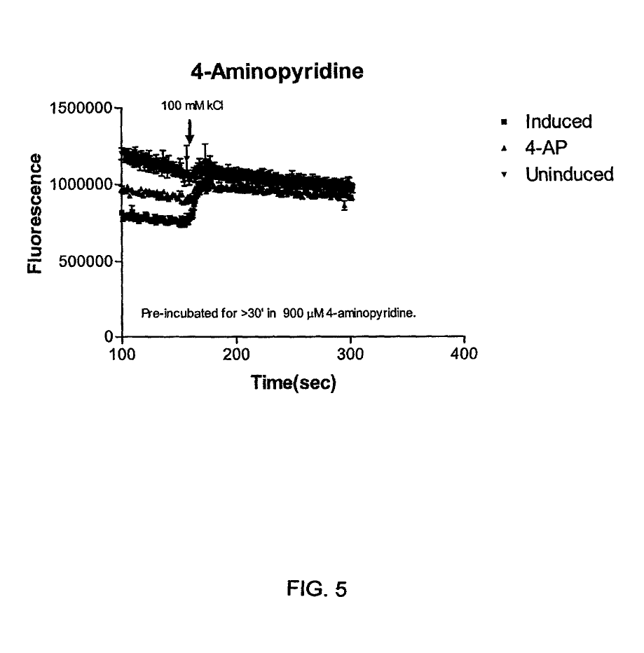

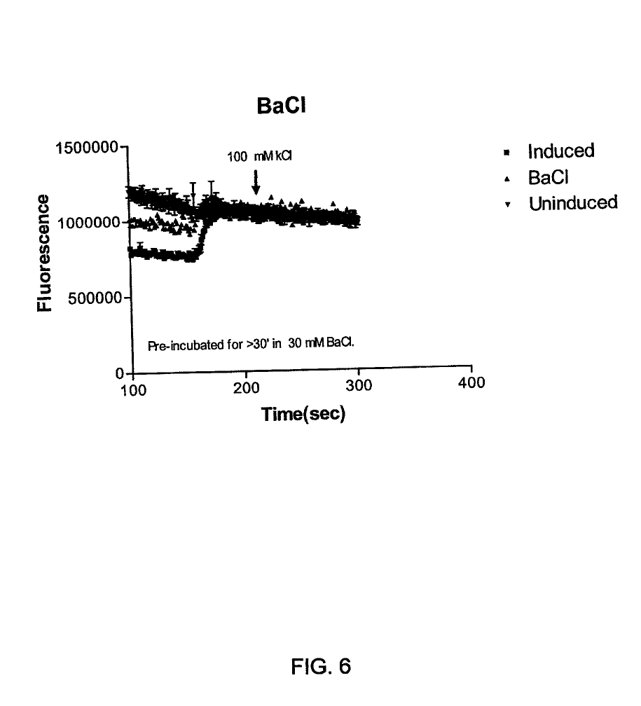

The addition of the KCNC1 inhibitor aminopyridine to the induced cells lessened the response to KCl addition (FIG. 5). BaCl₂, a K⁺ channel inhibitor, also ameliorated the response to KCl addition (FIG. 6).

Target-dependent responses may also be measured or observed by secondary effects that demonstrate the expression of the target sequence such as by measuring changes in cellular adhesion and may vary depending on the target sequence.

Expression of a G-protein coupled receptor at high levels generally causes activation of a functional response (Wess et al., J. Pharmacol. Ther. 80:231-264, 1998; Choi et al., J Neurosci Methods. 94:217-25, 2000). Consequently, when the target sequence comprises a G-protein coupled receptor coupled to Gi, an assay that measures a decrease in cellular cyclic AMP (“cAMP”) levels is desired. When the GPCR is coupled to Gs and is constitutively active and inducibly expressed, an assay that measures increases in cAMP levels is desired. Furthermore, when the GPCR is coupled to a Gq family G-protein, is constitutively active and inducibly expressed, an assay that measures intracellular calcium levels may be desired. Examples of techniques to measure cAMP levels are competitive binding assays (the Biotrak enzyme immunoassay (Wallac, Piscataway, N.J.)) or a Fluorescence polarization assay (NEN Life Science Products, Boston, Mass.)(Post et al., Methods Mol. Biol. 126:363-74, 2000).

Intercellular calcium levels may be detected by commercially available dyes such as Fura, Fluo or Indo (Molecular Probes, Eugene, Oreg.). These dyes bind to calcium and cause a shift in the absorbance of the dye (Palmer et al., Am. J. Physiol. 279, C1278, 2000; Collet et al., J. Physiol. 520: 417-429, 1999; Meth. Molec. Biol. 114, (David Lambert, ed. Humana Press), 1999; 376). Detecting a dye may be performed by flow cytometric analysis such as for example at 356/478 nm for indo-1.

When cAMP levels are assayed at least four daughter plates containing the construct may be used to test at least four conditions. The first plate is utilized as a control comprising transfected cells in which endogenous cAMP levels are measured. The second plate is utilized as a positive control and contains an agent, such as Forskolin, able to elevate endogenous cAMP levels. Preferably, the cAMP level is elevated to about 80% of maximum. This is determined by running a concentration range and monitoring the resulting cAMP levels. Maximum is the concentration at which the curve reaches a plateau. The third plate comprises an inducer able to induce transcription of the target sequence, and the cAMP level is monitored over time. The fourth includes the inducer and the test compounds. When the maximum induction of the target construct occurs, cAMP levels may be measured over time and may continue until returning to steady state. Recordings are made documenting the elevation or depression of cAMP in response to target induction in order to determine the optimum amount of inducer for each induction procedure. Cells that show changes in the level of cAMP greater than about three standard deviations of the population average following induction are sorted into multiwell plates and grown to multicellular colonies.

When calcium levels are assayed, two conditions are preferable. The first comprises transfected cells absent inducer, and the second comprises adding an inducer and measuring calcium levels by detecting the fluorescent properties of the calcium sensitive-dye over time using a fluorometer. Cells that show changes in the level of calcium dependent fluorescence greater than about three standard deviations of the population average following induction are sorted into multiwell plates and grown to multicellular colonies.

Induction of an ion channel target will generally increase the number of channels in the cell membrane and result in a change in membrane potential. Therefore, when the target is an ion channel, the assay preferably measures a change in membrane potential. Fluorescent dyes such as DIBAC (N4olecular Probes, Eugene, Oreg.) may detect changes in membrane potential (Epps et al., Chem. Phys. Lipids 69:137-150 1994; Waggoner, J Membr. Biol. 27:317-34, 1976).

When the target sequence is a nuclear hormone receptor or transcription factor, the direct phenotypic readout may be assayed by expression of an endogenous marker gene (Davis D. L. and Burch J. B., Mol. Endocrinol. 10:937-44, 1996) or by using a promoter-reporter construct (Martinez E. et al., EMBO J. 6:3719-27, 1987). The promoter-reporter construct may be any reporter sequence that is operably linked to a promoter and an enhancer sequence that is responsive to the receptor or transcription factor, such that when the promoter is active, the reporter verifies translation of the construct. For example luciferase may be linked to the HSV thymidine kinase minimal promoter and an estrogen response element. Briefly, when the promoter is activated by binding of the estrogen receptor to the response element, the enzymatic activity of luciferase in cell extracts may be detected upon addition of a suitable luciferase substrate (such as Luc-Lite, Packard Bioscience, Meriden, Conn.) by measurement of the light emitted.

Because receptors for growth factors, angiogenesis factors, or cytokines are known to couple through specific intracellular pathways to activate gene expression, the promoter-reporter strategy may also be useful in measuring activity. Growth factor or angiogenesis factor receptor activation may be measured either by autophosphorylation (Smaill J. B. et al., J. Med. Chem. 44:429-40, 2001), or by promoter-reporter constructs (Ghezzo F. et al., J. Biol. Chem. 263:4758-63, 1988). Cytokine receptor activation may be measured by phosphorylation of STAT proteins (Spiotto M. T. and Chung T. D., Prostate 42:88-98, 2000) or by STAT reporter constructs (Gaemers I. C. et al., J. Biol. Chem. 276:6191-9, 2001).

When the target sequence encodes a transporter, changes in intracellular pH may be measured to determine activity. Ion transporters such as proton pumps or anion transporters where hydrogen ions are accumulated within the cell, lead to a change in pH. For example, changes in activity of the sodium/hydrogen exchanger would alter the intracellular proton concentration. The activity of the sodium/hydrogen exchanger is coupled with the activity of other cation exchangers and thus intracellular pH is an indication of the activity of all cation exchangers. Intracellular pH may be measured by the detection of added dyes such as SNARF (Molecular Probes, Eugene, Oreg.) that change their optical properties in response to changes in pH. Dyes such as SNARF may be measured using flow cyomtetric anaylsis (Burchiel S. W. et al., Methods 21:221-30, 2000, van Erp P. E. et al., Cytometry 12:127-32, 1991).

When the target sequence encodes a protein that induces apoptosis such as by stimulation of the Fas receptor, different markers representing different points within the chain of cellular events may be measured such as activation of caspases (Smolewski P. et al., Cytometry 44: 73-82, 2001), display of cell surface markers, intracellular acidification, calcium mobilization, and changes in permeability. Dyes that change their optical properties in response to cellular pH, calcium, and membrane permeability such as SNARF (van Hooijdonk C. A. et al., Cell Prolif 30:351-363, 1997), FURA (Palmer B. M. and Moore R. L., Am. J. Physiol. 279:C1278 2000), and propidium iodide (Eray M. et al., J. Cytometry 43:134-142, 2001) may be used to detect activation. Preferably, the dyes fluoresce at different detectable wavelengths so that multiple independent measurements may be made simultaneously and detected using a flow cytometer or plate reader.

IV. Testing Compounds for the Ability to Modulate the Activity of an Induced Target Sequence Gene Product.

Once cells that selectively express the target sequence have been identified and the desired inducing conditions have been determined, cells are grown and assayed to determine the effects of potential modulatory compounds. Testing for modulation of the expressed target sequence occurs prior to induction and after induction. Testing may also occur once induction has ceased and the cell is allowed to return to its “steady state.”

Differences in the measurements between the “steady state” and “activated state” in the presence and absence of these compounds allows one to determine whether modulation has occurred.

A “steady state” measurement is taken prior to induction. The “steady state” measurement comprises cells transfected with inducible construct in the presence or absence of a potential modulator molecule compound. The concentration of the test cells in the assay are generally from about 1×10⁵ cells/mL to about 2×10⁶ cells/mL. However, depending on the cell lines selected, one skilled in the art would recognize that the choice of inducible constructs and assays may require routine optimization.

Cells may be plated into multiwell plates and inducer added. Potential modulatory compounds may be added at the time expression commences. Control wells within the plate may receive either no inducer or compound, or inducer with no compound. The data may be analyzed to determine whether any of the compounds tested cause a signal deviation greater than about 3 standard deviations from the control wells that receive only inducer. During testing the control wells are monitored to ensure that the target is expressed and functionally active. Compounds identified as having activity may be tested against non-induced cells in a second identical assay excluding inducer to ensure that their effects are target related, rather than having an affect on basal activity.

The inducer is added at a concentration that produces a measurable change in the expression of the target by testing for target-dependent responses. The target sequence is verified by methods previously described. In addition the concentration of inducer will depend on the cell line, the assay, and the construct as previously described.

“Activated state” measurements are compared to “steady state” measurements to determine whether the potential modulator molecule has modulated the expressed target sequence. For example, modulation of a G-protein coupled receptor may be demonstrated by a change in cAMP or cellular calcium levels during activation.

Compounds that test positive are then assayed to determine their effects on the induction mechanism to identify false positives. One method to identify false positives is to test the compounds on a control cell line. The control cell line is preferably of the same cell type as the test cell line and may comprise a reporter gene such as luciferase in place of the target sequence. If the reporter gene is inhibited luciferase will not be detected and it is likely that the compound is affecting the induction process and not the expressed target. When this occurs, the compound is no longer considered as a potential modulator molecule under the current test conditions.

In addition positive compounds may be tested against a family of proteins to determine their specificity for a particular member protein in that family. For example, Clozapine is known to inhibit D4 and 5HT2A/C receptors. In this configuration multiple constructs may be created where each expresses a G-protein coupled receptor and each transfected into a different cell.

The present invention may also be used to further define or study a biological pathway such as for example an enzymatic cascade pathway. More specifically one could place a regulatory kinase such as MAP kinase under inducible control. Induction of the kinase to high levels may activate the MAP kinase cascade. Alternatively, one may engineer many signaling molecules to be ‘dominant negative’ e.g. ‘kinase dead’ mutants where key catalytic residues of the enzyme are mutated, or isolated DNA binding domains of transcription factors. Inducible expression of these mutants may cause loss of function of the signaling pathway and may be useful in target validation studies.

V. A Kit for Identifying Modulatory Molecules

A kit for identifying modulatory molecules may be any kit comprising a cell line that conditionally expresses a target sequence and an inducer able to induce expression of a target. The kit may further comprise a fluorescent dye able to detect a change in a secondary effect that suggests binding of the target to a modulatory molecule, a buffered saline solution, and culture media.

The cell lines may be provided growing in microtitre plates or flasks at 37 C or frozen in vials or microtitre plates in liquid nitrogen. If frozen, the cells are thawed and resuspended in growth media. Standard growth media is provided with the cells and is typically DMEM+10% FCS. The membrane-potential sensitive dye is prepared as a stock solution in DMSO and is diluted in assay media. Preferred assay media is PSS+glucose or hybridoma media (Sigma, Saint Louis, Mo.).

When the target is an ion channel, the cell line may be CHO or HEK293, the fluorescent dye may be DIBAC, the buffered saline solution may be PBS, and the culture media may be DMEM. When the target is a receptor (GPCR, cytokine or nuclear hormone) the cell line may be CHO or HEK293, the fluorescent dye may be DIBAC or FURA, the buffered saline solution may be PBS, and the culture media may be DMEM.

The above disclosure generally describes the present invention. A more complete understanding can be obtained by reference to the following specific examples which are provided herein for purposes of illustration only and are not intended to limit the scope of the invention.

### EXAMPLES

### Example 1

### Insertion of the Mouse Potassium Voltage-Gated Channel KCNC1 Gene into the pcDNA4/TOb Inducible Expression Vector

Plasmid number 63333 (ATCC, Rockville, Md.) containing the mouse potassium voltage-gated channel KCNC1 cDNA, the mammalian expression vector pcDNA4/TOb (Invitrogen, Carlsbad, Calif.) were commercially obtained. Both were digested with the restriction enzymes KpnI and PstI (New England Biolabs, Beverly, Mass.). The 2 kb KCNC1 gene fragment and the pcDNA4/Tob vector were gel purified, ligated and transformed into competent Top10F′ E. coli (Invitrogen, Carlsbad, Calif.). Positive clones were identified by restriction analysis of plasmid DNA and confirmed by DNA sequencing. Plasmid DNA for transfection was prepared with an Endotoxin free kit (Qiagen, Valencia, Calif.).

### Example 2

### Transfection of the Inducible Expression Vector Target Construct

The pcDNA4/Tob/KCNC1 plasmid (FIG. 1) was transfected into T-Rex CHO cells (Invitrogen, Carlsbad, Calif.) by the following procedure. Cells were seeded into a 6-well plate at 2×1 5 cells per well. The next day cells were transfected using FuGene Reagent (Roche, Indianapolis, Ind.). The following morning transfected cells were split 1:10 into a 10 cm plate. Twenty-four hours later selection in 400 μg/mL zeocin (Invitrogen, Carlsbad, Calif.) was initiated, and continued for two weeks. Individual colonies of zeocin resistant cells were isolated using cloning paper (Scienceware, Pequannock, N.J.) and passaged into a 24 well plate.

When cells became confluent, the clones were split in triplicate among 24-well plates. To identify clones that were able to express KCNC1, One set of clones was induced to express KCNC1 with 10 ug/mL tetracycline for 24 hours before cells were processed for immunohistochemistry. An identical set of non-induced clones was also processed for immunohistochemistry. Clones producing the KCNC1 protein were identified using an affinity-purified rabbit antibody to Kv3.1b (Sigma, St. Louis, Mo.), the rat homologue of the mouse KCNC1 (NEB, Ontario, Canada), and a secondary goat-anti rabbit antibody conjugated to horseradish peroxidase (NEB, Ontario, Canada). The assay was developed using TrueBlue Peroxidase Substrate (KPL Inc., Gaithersburg, Md.). Clones that expressed KCNC1 in 100% of the cell population when induced and in 0% of the cell population when not induced were saved and expanded in a third 24-well plate. All clones were maintained in zeocin.

### Example 3

### Confirming the Induction of the Mouse Potassium Voltage-Gated Channel KCNC1 Gene

Induction of the KCNC1 gene was confirmed by RT-PCR analysis of mRNA and by immunohistochemistry.

PCR was used to verify production of KCNC1 mRNA (FIG. 2). Two samples each containing 2×10⁴ cells were collected from clones 7, 13, and 22. The first sample was a control whereby there was no induction and the second sample was induced with 10 μg/mL of tetracycline. The mRNA was reverse-transcribed into cDNA using SuperScriptII (Invitrogen, Carlsbad, Calif.). PCR was performed in a GeneAmp 9600 thermocycler (Applied Biosystems, Foster City, Calif.) using a forward primer (5′-CCACCAGACGTACCGCTCATC-3′) and reverse primer (5′CGGTGCTGGCGATAGGTCATC-3′) specific for the expressed KCNC1 sequence. PCR products were separated on a 1.5% agarose gel and stained with SYBR Gold (Molecular Probes, Eugene, Oreg.). KCNC1 induction was detected in induced cells but was absent in non-induced cells.

The stable, zeocin-resistant cell lines containing the KCNC1 gene were once again tested for their ability to produce the KCNC1 protein upon induction (FIG. 3), following essentially the same method as described in Example 2, above.

### Example 4

### Method of Screening and Identifying a Modulator Molecule for an Ion Channel

A membrane potential assay demonstrated depolarization of the an induced population of cells in comparison to a non-induced cell population upon the addition of potassium chloride in 50 mM steps (FIG. 4). A KCNC1 positive TREX/CHO clone was plated at 3×10⁶ cells in replicate 10 cm tissue culture dishes. After 24 hours one dish was treated with 10 μg/mL deoxycycline to induce KCNC1 expression. After a 24 hour induction period, both induced and uninduced cells were harvested with trypsin, counted, and adjusted to equal cell densities in hybridoma media (Sigma, St. Louis, Mo.). A solution of 3.3×10⁵ cells and 0.4 μM of each Disbac5Me4 and Disbac3Me4 in hybridoma media was stirred in a cuvette in a JY-Fluormax-2 fluorometer (JY, Edison N.J.). Fluorescence intensity from 540 nm excitation and 690 nm emission was measured over time. The extracellular potassium chloride level was adjusted to 50 mM, 10 mM, and 150 mM with 3 N KCl at the indicated times. Each cell population was tested in triplicate and the mean and standard error (SE) were determined.

To demonstrate inhibition of KCNC1, the inhibitors 4-aminopyridine (900 μM) and BaCl₂ (30 mM) were pre-incubated with cells at least 30 minutes prior to addition of membrane potential dyes and fluorescence measurement. 4-aminopyridine is a known specific inhibitor of Kv3.1b (Grissmer et al., Molec. Pharmacol. 45:1227-1234, 1994; Kirsch and Drewe, Jour. Gen. Physiol. 102:797-816, 1993; Grissmer et al. Jour. Biol. Chem. 267:20971-20979, 1992), the human homologue of KCNC1. BaCI₂, another known inhibitor of K⁺ channels (Lopes et al, J. Biol. Chem. 276:24449-52, 2001; Clarson et al., Placenta 22:328-36, 2001), also results in a less polarized resting potential and a decreased response to depolarization with KCl, as shown in FIG. 6. Pre-incubation with 30 mM KCl had no effect, ruling out the possibility that effects of BaCl₂ resulted from simply changing the ionic strength of the extracellular medium (data not shown). Each cell population was tested in triplicate. The mean and SE are shown in the FIG. 5 (aminopyridine) and FIG. 6 (BaCl₂).

### Example 5

### Transfection and Testing of an Inducible Expression Vector Construct Containing a HERG-Encoding Gene

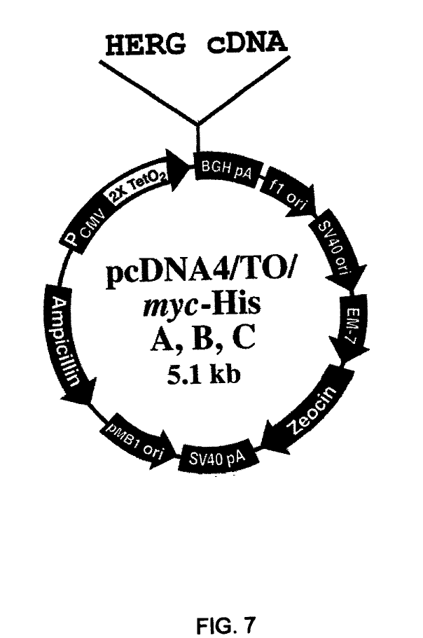

The pcDNA4/TOb/HERG plasmid (FIG. 7) was transfected into T-REx CHO cells (Invitrogen, Carlsbad, Calif.). Cells were seeded into a 6-well plate at 2×10⁵ cells per well. The next day cells were transfected using FuGene Reagent (Roche, Indianapolis, Ind.). The following morning transfected cells were split 1:10 into a 10 cm plate. Twenty four hours later selection in 400 mg/ml zeocin (Invitrogen, Carlsbad, Calif.) was begun, and continued for two weeks.

Individual colonies of zeocin resistant cells were isolated using cloning paper (Scienceware, Pequannock, N.J.) and passaged into a 24-well plate. When cells became confluent the clones were split in triplicate among 24-well plates. One set of clones was induced to express HERG with 10 mg/ml tetracycline for 24 hours before cells were processed for immunohistochemistry. An identical set of non-induced clones was also processed for immunohistochemistry. HERG expressing clones were identified using an affinity-purified rabbit antibody to HERG (Alomone Labs, Jerusalem, Israel). A secondary goat-anti-rabbit antibody conjugated to horseradish peroxidase (NEB, Ontario, Canada) was then detected using TrueBlue Peroxidase Substrate (KPL Inc., Gaithersburg, Md.). Clones that expressed HERG in 100% of the cell population when induced and in 0% of the cell population when not induced were saved and expanded from the third 24-well plate. All clones were maintained in zeocin selection.

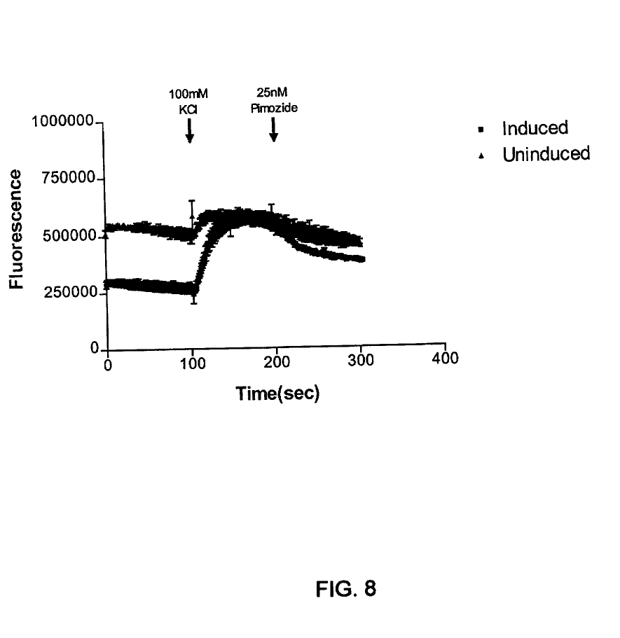

The HERG positive TREX/CHO clone 5J was plated at 3×10⁶ cells in replicate 10 cm tissue culture dishes. After 24 hours one dish was treated with 10 mg/ml doxycycline to induce HERG expression. After 24 hours induction, both induced and uninduced cells were harvested with trypsin, counted and adjusted to the same cell density in hybridoma media (Sigma). A solution of 1×10⁵ cells/ml and 0.4 μM each Disbac5Me4 and Disbac3Me4 in hybridoma media was stirred in a cuvette in a JY-Spex fluorometer. Fluorescence intensity from 540 excitation and 690 emission was followed over time. The extracellular potassium chloride was adjusted to 100 mM with 3N KCl at the indicated time. 25 nM pimozide was then added at the indicated time. Each cell population was tested in triplicate and the mean and SE are shown in FIG. 8.

### Example 6

### Construction of an Homologous Recombination Vector Construct

The creation of the inducible target gene can be accomplished by a number of strategies, including the use of homologous recombination to replace a specific endogenous regulatory region of a gene with an inducible regulatory region. In a typical homologous recombination strategy, an adaptor fragment is introduced into the genome of recipient cells for insertion of a regulatory region upstream of the coding region of the target gene. Specifically, the targeting construct from which this fragment is derived is designed to include a first targeting sequence homologous to sequences upstream of the target gene, a selectable marker gene, an inducible regulatory region, and a second targeting sequence corresponding to sequences downstream of the first targeting sequence but upstream of exon 1 of the target gene. This strategy allows the endogenous promoter of a target gene to be replaced with an inducible promoter. The resulting homologously recombinant cells can be induced to produce an mRNA transcript of the target gene.

For example, a homologous recombination vector containing the inducible promoter and the targeting sequences of a given target gene may be constructed by the following method. A restriction enzyme digestion of a subcloning vector such as pBS (Stratagene, Inc., La Jolla, Calif.) containing the genomic DNA sequences within 1-5 kb of coding regions of the gene of interest is designed (based on the restriction map of the target gene upstream region and data published from human genome sequencing) in order to isolate the desired DNA fragments corresponding to 1) an upstream homologous recombination target sequence 1 of the given gene, and 2) an upstream homologous recombination target sequence 2 of the given gene. The upstream fragments are then sequentially ligated to the plasmid containing the inducible promoter construct, so that the inducible promoter construct is between recombination target sequence 1 and 2. Optionally, one or more selectable marker genes may be added to the construct. The plasmid is then transformed into competent E. Coli cells or other cells, including human cell lines, and colonies containing the above inserts are analyzed by restriction enzyme analysis to confirm the orientation of the insert.

### Example 7

### Method of Screening and Identifying a Modulator Molecule for an Endogenous Ion Channel Protein Using a Homologous Recombination Vector Construct

An inducible promoter and selectable marker are inserted by homologous recombination into a human tumor cell line that contains an endogenous copy of KCNC1, and transformed cells are selected using conventional techniques.

A membrane potential assay is then conducted using various candidate modulator molecules, by repeating the steps of Example 4 for each candidate molecule.

What is claimed is:

1. A method for identifying compounds that modulate a target protein, comprising:
providing cells transfected in such a way as to provide a polynucleotide sequence encoding said target under control of a heterologous inducible promoter;
inducing the promoter under conditions that provide a detectable change in a measurable parameter associated with the cells;
contacting at least a portion of the cells with a test compound to ascertain whether the test compound affects a change in the measurable parameter; and
repeating the contacting step with at least one other test compound.

2. The method of claim 1, wherein the measurable parameter is a parameter other than growth or survival.

3. The method of claim 1, wherein the contacting step comprises contacting cells with said test compound while the promoter is induced.

4. The method of claim 1, further comprising comparing the value of the measurable parameter in uninduced cells with the value of the parameter in induced cells.

5. The method of claim 4, wherein the measurable parameter has been selected from among a plurality of candidate parameters based on said comparison.

6. The method of claim 1, wherein the promoter is induced to a degree that provides a detectable change in the parameter but not to a degree that kills the cell.

7. The method of claim 1, wherein the promoter is induced by contacting the cell with an inducer molecule.

8. The method of claim 1, wherein the promoter is induced by removal or inhibition of a repressor.

9. The method of claim 1, wherein the target protein affects ion channel activity of the cell.

10. The method of claim 9, wherein the target protein is an ion channel protein.

11. The method of claim 9, further comprising:
identifying at least one test compound that modulates the measurable parameter in the cell;
providing a second cell line that differs from the first cell line in that the inducible promoter controls expression of a reporter instead of polynucleotide encoding target;
contacting the second cell line with the identified test compound; and
ascertaining whether the identified test compound affects the expression of the reporter.

12. The method of claim 1, wherein said polynucleotide encoding target and said promoter have been transfected into a mammalian cell.

13. The method of claim 1, wherein said inducible promoter replaces an endogenous promoter and controls the expression of an endogenous polynucleotide encoding target.

14. A method for identifying an ion channel modulator molecule comprising the steps of:
a. obtaining a cell that conditionally expresses an ion channel target;
b. incubating a potential ion channel modulator molecule with said cell; and
c. determining whether ion flow through said ion channel targets has modulated, thereby identifying molecules that modulate said ion channel target.

15. A method according to claim 14 wherein said cell that conditionally expresses said ion channel target has been induced to express said ion channel target.

16. A method according to claim 14 wherein said cell is selected from the group consisting of CHO, CHO-K1, HEK293, COS, Vero, SH-SY5Y, RBL and U20S.

17. A method according to claim 14 wherein the step of obtaining a cell that conditionally expresses an ion channel target comprises genetically adapting said cell to produce an ion channel target.

18. A method according to claim 17 wherein said cell is genetically adapted by transducing or transfecting said cell with an inducible vector comprising an ion channel target.

19. A method according to claim 18 wherein said inducible vector comprises an inducible cassette wherein said inducible cassette comprises an inducible promoter, an ion channel gene, and a gene conferring resistance to a selection agent for selecting transfected cells wherein said inducible promoter is operably linked to said ion channel gene.

20. A method according to claim 19 wherein said inducible promoter is selected from the group consisting of the heat shock inducible promoter, metallothionin promoter, ecdysone-inducible promoter, FKBP dimerization inducible promoter, Gal4-estrogen receptor fusion protein regulated promoter, lac repressor, steroid inducible promoter, streptogramin responsive promoters and tetracycline regulated promoters.

21. A method according to claim 18 wherein said inducible vector may be activated to express said ion channel target and inactivated to prevent expression of said ion channel target.

22. A method according to claim 14 wherein said ion channel target is an ion channel selected from the group consisting of a sodium ion channel, an epithelial sodium channel, a chloride ion channel, a voltage-gated chloride ion channel, a potassium ion channel, a voltage-gated potassium ion channel, a calcium-activated potassium channel, an inwardly rectifying potassium channel, a calcium ion channel, a voltage-gated calcium ion channel, a ligand-gated calcium ion channel, a cylic-nucleotide gated ion channel, a hyperpolarization-activated cyclic-nucleotide gated channel, a water channel, a gap junction channel, a viral ion channel, an ATP-gated ion channel and a calcium permeable beta-amyloid peptide channel.

23. A method for identifying an ion channel modulator molecule, comprising the steps of:
a. obtaining a cell that conditionally expresses an ion channel target;
b. adding an inducer molecule that induces expression of said ion channel target in said cell;
c. measuring membrane potential of said cell;
d. incubating a potential ion channel modulator molecule with said cell;
e. measuring changes in membrane potential; and
f. determining whether ion flow through said ion channel targets has been modulated, thereby identifying a molecule that modulates said ion channel.

24. A method for screening chemical compounds to identify an ion channel modulator compound, comprising the steps of:
a. obtaining a cell that conditionally expresses an ion channel target;
b. adding an inducer molecule that induces expression of said ion channel target in said cell;
c. measuring membrane potential of said cell;
d. incubating said chemical compounds with said cell; and measuring changes in membrane potential;
e. determining whether ion flow through said ion channel targets has been modulated, thereby identifying compounds that modulate said ion channel target.

25. A method for identifying a membrane receptor modulator molecule comprising:
a. obtaining a cell that conditionally expresses a target membrane receptor;
b. inducing expression of said target membrane receptor;
c. measuring a physiological condition of said cell to obtain a first set of data;
d. incubating a potential membrane receptor modulator molecule with said cell;
e. measuring said physiological condition of said cell to obtain a second set of data; and
f. comparing said first set of data with said second set of data to determine whether said physiological condition of said cell has been modulated, thereby identifying a molecule that modulates said target membrane receptor.

26. A method according to claim 25 wherein the step of obtaining a cell that conditionally expresses said membrane receptor comprises:
a. obtaining a cell that contains an endogenous target membrane receptor sequence and an endogenous noncoding sequence; and
b. inserting an inducible cassette comprising a 5′ insertion adapter, a regulatory sequence and a 3′ insertion adapter within said endogenous noncoding sequence such that said regulatory sequence is operably linked such that it is able to modulate transcription of said target membrane receptor by the presence or absence of a regulator.

27. A method according to claim 26 wherein said regulatory sequence is a non-mammalian enhancer sequence or a repressor sequence.

28. A method according to claim 27 wherein said non-mammalian enhancer sequence is a herpes virus enhancer or an artificial enhancer.

29. A method according to claim 28 wherein said non-mammalian enhancer sequence is an inducible promoter.

30. A method according to claim 29 wherein said inducible promoter is a herpes virus promoter.

31. A method according to claim 29 wherein said inducible cassette further comprises a target sequence such that said target sequence is transcribed upon induction of said inducible cassette.

32. A method according to claim 31 wherein said target sequence is selected from the group consisting of a G-protein coupled receptor target sequence, a nuclear hormone receptor target sequence, a cytokine receptor target sequence, a protein kinase-coupled receptor target sequence a nicotinic acetylcholine receptor target sequence, a ionotropic glutamate receptor target sequence, a glycine receptor target sequence, a gamma-aminobutyric acid receptor target sequence, and a vanilloid receptor target sequence.

33. A method according to claim 32 wherein said target sequence is 5HT4.

34. A method according to claim 27 wherein said repressor sequence is able to bind a zinc finger protein.

35. A method according to claim 34 wherein said zinc finger protein comprises a KRAB domain.

36. A method according to claim 26 wherein said regulator is VP16 or a functional domain of VP16.

37. A method according to claim 25 further comprising transfecting said cell with a regulatory expression vector construct comprising a second inducible promoter and a regulator gene encoding said regulator operably linked such that induction of said second inducible promoter by an exogenous stimulus initiates transcription of said regulator gene.

38. A method according to claim 37 wherein said second inducible promoter is a tetracycline inducible promoter or an ecdysone-inducible promoter.

39. A method according to claim 37 wherein said exogenous stimulus is tetracycline, ponasterone, dexamethasone, a heavy metal ion or heat.

40. A method according to claim 25 wherein said step of inducing expression of said target membrane receptor is initiated by the presence or absence or a regulator or by the presence or absence of an inducer.

41. A method for screening a chemical compound library to identify a G-protein coupled receptor modulator molecule, comprising:
a. obtaining a cell that conditionally expresses a G-protein coupled receptor;
b. inducing expression of said G-protein coupled receptor;
c. measuring a physiological parameter associated with said G-protein coupled receptor to obtain a first set of data;
d. incubating a potential modulator of said G-protein coupled receptor with said cell;
e. measuring said physiological parameter to obtain a second set of data; and
f. comparing said first set of data with said second set of data to determine whether said physiological parameter has been modulated, thereby identifying a chemical compound that modulates a G-protein coupled receptor.

42. A method according to claim 41 wherein said physiological parameter is selected from the group consisting of a cAMP level, a calcium level, and a membrane potential of said cell.

43. An inducible vector containing an ion channel target having a nucleotide sequence shown in SEQ. ID NO.: 1.

44. An inducible expression vector comprising a tetracycline inducible promoter, a pcDNA4/TO vector construct and a human HERG potassium channel gene.

45. An inducible regulatory expression vector construct comprising a subcloning vector, a second inducible promoter and a regulator gene.

46. A cell transduced or transfected with the inducible vector of claim 44.

47. A cell transduced or transfected with the inducible vector according to claim 46 wherein said cell is a CHO cell and wherein said transduced or transfected cell expresses Tet repressor and HERG potassium ion channel gene.

48. An ion channel modulator molecule identified by the method according to claim 14.

49. A membrane receptor modulator molecule identified by the method according to claim 25.

50. A G-protein coupled receptor modulator molecule identified by the method according to claim 41.

51. A kit comprising cells that conditionally express an ion channel target, a compound that induces expression of the ion channel target, and an indicator compound or system for indicating ion channel activity of said cells.

52. A kit comprising cells that conditionally express an ion channel target and a fluorescent dye.
